(19) United States
(12) Patent Application Publication
(10) Pub. No.: US 2024/0285543 A1
(43) Pub. Date: Aug. 29, 2024

(54) NANOPARTICLE-MEDIATED ENHANCEMENT OF IMMUNOTHERAPY TO PROMOTE FERROPTOSIS-INDUCED CYTOTOXICITY AND ANTITUMOR IMMUNE RESPONSES

(71) Applicants: Memorial Sloan-Kettering Cancer Center, New York, NY (US); Sloan-Kettering Institute for Cancer Research, New York, NY (US); Memorial Hospital for Cancer and Allied Diseases, New York, NY (US); Cornell University, Ithaca, NY (US); The Curators of the University of Missouri, Columbia, MO (US)

(72) Inventors: Michelle S. Bradbury, Ithaca, NY (US); Michael Overholtzer, Glen Ridge, NJ (US); Virginia Aragon, New York, NY (US); Gabriel DeLeon, Ithaca, NY (US); Ulrich Wiesner, Ithaca, NY (US); Thomas P. Quinn, Columbia, MO (US); Michael R. McDevitt, New York, NY (US)

(21) Appl. No.: 18/572,423

(22) PCT Filed: Jun. 21, 2022
(86) PCT No.: PCT/US22/34224

§ 371 Date: Dec. 20, 2023

Related U.S. Application Data
(60) Provisional application No. 63/212,930, filed on Jun. 21, 2021.

(51) Int. Cl. A61K 9/51; A61K 9/00; A61K 31/519; A61K 39/00; A61K 39/395; A61K 47/69; A61K 51/08; A61N 5/10; A61P 35/00; C12Q 1/6886

(52) U.S. Cl. CPC A61K 9/5115; A61K 9/0019; A61K 31/519; A61K 39/3955; A61K 39/4611; A61K 39/4631; A61K 47/6929; A61K 51/088; A61N 5/10; A61P 35/00; C12Q 1/6886; A61N 2005/1089; A61N 2005/109; A61N 2005/1098; C12Q 2600/158

(57) ABSTRACT

Described herein are methods of treating cancer by administering to a subject a composition comprising ultrasmall silica nanoparticles to enhance one or more of the following immunotherapies: chimeric antigen receptor (CAR) T-cell therapy, immune checkpoint blockade antibody therapy (ICB), immune inhibitor therapy (e.g., myeloid-targeting inhibitors). In some embodiments, the compositions are used in combination with external beam radiotherapy.

### CROSS REFERENCE TO RELATED APPLICATION

This application claims the benefit of U.S. Provisional Application No. 63/212,930, filed Jun. 21, 2021, entitled “Nanoparticle-Mediated Enhancement of Immunotherapy to Promote Ferroptosis-Induced Cytotoxicity and Antitumor Immune Responses,” the disclosure of which is hereby incorporated by reference in its entirety.

### GOVERNMENT SUPPORT

This invention was made with government support under grant numbers CA199081, CA253658, and CA008748, awarded by the National Institutes of Health. The government has certain rights in the invention.

### FIELD OF THE INVENTION

This invention relates generally to methods and compositions for the treatment of cancer in subjects. More specifically, in certain embodiments, the invention relates to enhancing immunotherapeutic responses for the treatment of cancer.

### BACKGROUND OF THE INVENTION

Identifying new therapies that can eliminate cancer has been a significant research and clinical goal for decades. Cancer cells have traditionally been targeted with pharmacological agents that are either preferentially cytotoxic to dividing cells, or that block specific cancer-activated pathways to inhibit division or induce cell death. Such treatments, which generally induce apoptosis with or without unregulated necrosis, are associated with significant toxic effects on normal tissues or fail to eliminate all cells within cancerous lesions, limiting efficacy and promoting tumor recurrence. While immune checkpoint blockade and engineered cellular therapies (e.g., chimeric antigen receptor (CAR) T cells) have yielded dramatic responses in hard-to-treat tumors, their use is limited by ineffective solid tumor tissue penetration, off-target effects in immunosuppressed tumor microenvironments, and/or toxic side-effect profiles. Moreover, while immunotherapies have revolutionized the treatment of multiple advanced-stage malignancies, leading to durable responses and improvements in survival outcomes, this is only true for a relatively small subset of patients. In particular, a substantial fraction of treated patients develop immune-related adverse events. Given such treatment failures, it is critically important to develop improved therapeutic tools that can significantly reverse immune suppressive activities within the tumor microenvironment (TME) in order to overcome resistance to cancer immunotherapies and/or expand/sustain the treatment window for CAR T cell administration.

### SUMMARY OF THE INVENTION

Described herein are methods of treating cancer by administering to a subject a composition comprising ultrasmall silica nanoparticle compositions with intrinsic therapeutic properties to enhance one or more cancer immunotherapies, including engineered cellular therapies, such as chimeric antigen receptor (CAR) T-cell therapy, immune checkpoint blockade (ICB), and/or selective small molecule inhibitors targeting immune cells (e.g., myeloid-targeting inhibitors).

Recent discoveries focus on a unique set of multiple self-therapeutic capabilities of the particles themselves without the need for attached cytotoxic drugs including: (i) nanoparticle (e.g., C′ dot)-induced ferroptosis, in which particles actively engage an iron-driven mechanism of cancer cell death, and (ii) priming/activating immune cells directly within the tumor microenvironment (TME), which internalize these particles. These discoveries have led to new methods that harness the potent inherent anti-cancer activities of these particle-based tools to address limitations of cancer immunotherapies, while enhancing their therapeutic effectiveness in solid tumor malignancies. Furthermore, as CD8+ T cells are known to also regulate ferroptosis during immunotherapy, such effects are expected to synergize with particle-induced biological responses.

Moreover, the described nanoparticles offer a distinct combination of multiple separable anti-tumor intrinsic therapeutic activities in tumor models that (1) effectively modulate the TME toward a pro-inflammatory phenotype from one that is immunosuppressive, (2) increase anti-tumor immune cell activation and cytotoxicity in the TME, and (3) target cancer cells directly via one or more potent forms of cell death, including ferroptosis. The ability of a particle platform itself, without attached cytotoxic drugs, to modulate a suppressive phenotype toward a more inflammatory one represents a clear paradigm shift from traditional nanomedicine approaches which often need to encapsulate cytokines or adjuvants to improve efficacy or immunotherapeutic responses. Additionally, among efforts to identify mechanisms of cell death with relevance to human disease, ferroptosis is unique in that it promotes spreading of cell death throughout many murine and human cancer cell populations—an activity that has been proposed to be of clinical significance for eliminating malignant lesions. The described nanoparticles engage this form of cell death without associated off-target effects often reported with other platforms, and which seek to leverage this specific particle-driven, death-inducing activity for cancer therapy.

The methods described herein employ particle-mediated enhancement of anti-tumor immunity and cell death in immunosuppressed tumor microenvironments (TME). The methods can improve treatment outcomes under conditions of tumor-induced immunosuppression and immune resistance by harnessing the favorable biological properties of a set of self-therapeutic ultrasmall (e.g., no greater than 20 nm in diameter, e.g., no greater than 10 nm in diameter) diameter less than 8 nm) silica-organic core-shell nanoparticles with tunable size, composition, surface chemical properties, and functionalities, that have been translated to the clinic. Used as multitherapeutic treatment tools in combination with cancer immunotherapy, these particles (i) augment the capabilities of immunotherapeutic agents, e.g., yielding synergistic responses, (ii) improve properties of the tumor microenvironment that limit efficacious outcomes relative to immunotherapy alone, which limit efficacious outcomes by increasing activated T cell and antigen-presenting cell populations and decreasing inhibitory T cell-myeloid populations, and (iii) induce significant tumor growth reduction and/or regression relative to vehicle controls.

In certain embodiments, the administered nanoparticles exhibit and promote intrinsic pro-inflammatory and cancer cell death-inducing properties.

In some embodiments, the described nanoparticles can be administered to a subject prior to, simultaneously with, or after administering to the subject a composition comprising engineered cellular therapies, such as chimeric antigen receptor (CAR) T-cell therapy, immune checkpoint blockade (ICB), and/or selective small molecule inhibitors targeting immune cells.

Furthermore, the silica nanoparticles described herein are found to modulate multiple class-specific gene expression signatures across a variety of cancer cell types. For example, class-specific up-regulation of many gene expression signatures was observed for particle-exposed cell lines, as against media controls. Cytokine release assays, immunophenotyping, bulk RNAseq, immunohistochemistry/immunofluorescence, and single-cell RNA sequencing (scRNA-seq) can be used to further identify immunomodulation capabilities of silica nanoparticles on cell population dynamics in the TME, to discriminate among heterogeneous cell populations within the TME, and/or to identify rare cell populations that respond to the silica nanoparticles and play a role in therapeutic outcomes.

In one aspect, the invention is directed to a method of treatment of a subject having been diagnosed with cancer (e.g. prostate cancer, ovarian cancer, a malignant brain tumor (e.g., brain or spinal), melanoma) the method comprising administering (e.g., via IV administration) to the subject a composition comprising ultrasmall nanoparticles in concert with administering one or more of (i) to (iv) as follows: (i) cellular therapy (e.g., dendritic cell therapies, e.g., engineered cellular therapies); (ii) one or more immune checkpoint blockade antibodies (ICB) (e.g., ICB can synergize with particle-induced cytotoxicity (e.g., ferroptosis) and enhanced effector cell responses in the TME); (iii) one or more pharmacologic inhibitors; and (iv) external beam radiation or molecular radiotherapy (e.g., peptide radioligands (e.g., radiolabeled PSMA-targeting ligands)).

In some embodiments, the nanoparticles in combination with radiation enhance efficacy of checkpoint blockade. In some embodiments, the molecular radiotherapy comprise a radiotherapeutic label. In some embodiments, the radiotherapeutic label comprises an alpha-emitting radioisotope or a beta-emitting radioisotope. In some embodiments, the alpha-emitting radioisotope comprises ²²⁵Ac. In some embodiments, the beta-emitting radioisotope comprises ¹⁷⁷Lu. In some embodiments, the cell therapy comprises T-cell therapy or engineered cell therapy (e.g., CAR T cell therapy) (e.g., CAR T cell cytotoxicity can be promoted by particle-driven immunogenicity and immune cell priming/activation in ovarian cancer models). In some embodiments, the pharmacologic inhibitors comprise one or more myeloid cell-targeting inhibitors e.g., inhibition of suppressive myeloid cell populations can be augmented by particle-driven cell death programs and pro-inflammatory responses to treat melanoma.

In another aspect, the invention is directed to a method of treatment of a subject having been diagnosed with cancer (e.g., ovarian cancer), the method comprising: (i) administering (e.g., via IV administration) to the subject a composition comprising ultrasmall nanoparticles; and (ii) administering to the subject cells (e.g., T cells, e.g., dendritic cells, e.g., CAR T cells, e.g., engineered cells (e.g., engineered immune cells)).

In some embodiments, the cells comprise CAR-T cells (e.g., in a composition separate from the nanoparticle composition or in the same composition as the nanoparticle composition). In some embodiments, the nanoparticles (i) augment intratumoral immune responsiveness and cytotoxicity and/or (ii) improves CAR T cell exhaustion or enhances CAR T cell persistence. In some embodiments, the nanoparticles comprise tumor-targeting ligands. In some embodiments, the nanoparticles do not comprise tumor-targeting ligands.

In another aspect, the invention is directed to a method of treatment of a subject having been diagnosed with cancer (e.g., tumors comprised of high levels of myeloid cells, a main driver of immune evasion e.g., melanoma or triple negative breast cancer, TNBC), the method comprising: (i) administering (e.g., via IV administration) to the subject a composition comprising ultrasmall nanoparticles, said nanoparticles comprising targeting ligands; (ii) administering to the subject a myeloid-targeting inhibitor; and (iii) administering to the subject one or more immune checkpoint blockade antibodies.

In some embodiments, the cancer comprises one or more tumors comprised of high levels of myeloid cells. In some embodiments, the targeting ligands comprise melanocortin-1 receptor (MC1-R) targeting ligands of a single type or multiple types. In some embodiments, the myeloid-targeting inhibitor comprises a PI3Kγ-selective inhibitor targeting myeloid cells (e.g., IPI-549). In some embodiments, the one or more immune checkpoint blockade antibodies comprises a member selected from the group consisting of ipilimumab (anti-CTLA4), pembrolizumab (anti-PD1), nivolumab (anti-PD1), and atezolizumab (anti-PD-L1). In some embodiments, resistance to immune ICB is limited by combining particle-driven cytotoxic responses (e.g., ferroptosis, immune-related cell death) and enhanced pro-inflammatory responses with one or more immune checkpoint blockade antibodies and/or selective PI3Kγ-targeting to subvert immunosuppressive components in a tumor microenvironment (TME).

In another aspect, the invention is directed to a method of treatment of a subject having been diagnosed with cancer (e.g., prostate cancer), the method comprising: (i) administering (e.g., via IV administration) to the subject a composition comprising ultrasmall nanoparticles, said nanoparticles comprising targeting ligands; (ii) administering to the subject external beam radiotherapy or molecular radiotherapy (e.g., peptide radioligands (e.g., radiolabeled PSMA-targeting ligands)); and (iii) administering to the subject one or more immune checkpoint blockade antibodies.

In some embodiments, the targeting ligands comprise PSMA-targeting ligands of a single type or multiple types. In some embodiments, the molecular radiotherapy comprise a radiotherapeutic label. In some embodiments, the radiotherapeutic label comprises an alpha-emitting radioisotope or a beta-emitting radioisotope. In some embodiments, the alpha-emitting radioisotope comprises ²²⁵Ac. In some embodiments, the beta-emitting radioisotope comprises ¹⁷⁷Lu.

In some embodiments, the one or more immune checkpoint blockade antibodies comprises a member selected from the group consisting of ipilimumab (anti-CTLA4), pembrolizumab (anti-PD1), nivolumab (anti-PD1), and atezolizumab (anti-PD-L1). In some embodiments, the method further comprises administering to the subject a myeloid-targeting inhibitor. In some embodiments, the myeloid-targeting inhibitor comprises a PI3Kγ-selective inhibitor targeting myeloid cells (e.g., IPI-549).

In some embodiments, the nanoparticles have a diameter no greater than 20 nm. In some embodiments, the nanoparticles have a diameter no greater than 10 nm. In some embodiments, the nanoparticles comprise silica.

In some embodiments, the cancer comprises prostate cancer, ovarian cancer, malignant brain tumors, melanoma, breast cancer, or lung cancer.

In some embodiments, each of the nanoparticles comprises 1 to 25 targeting ligands (e.g., of a single species) (e.g., 2 to 20 ligands, 5 to 15 ligands, 5 to 10 ligands, or about 6-8 ligands). In some embodiments, the targeting ligand is a targeting ligand for a cellular receptor. In some embodiments, the targeting ligand for a cellular receptor comprises MC1-R or PSMA.

In some embodiments, each of the nanoparticles has a hydrodynamic diameter no greater than 20 nm (e.g., wherein the hydrodynamic diameter is in a range from 1 nm to 20 nm). In some embodiments, each of the nanoparticles has a hydrodynamic diameter no greater than 10 nm (e.g., wherein the hydrodynamic diameter is in a range from 1 nm to 10 nm). In some embodiments, each of the nanoparticles comprises a silica core. In some embodiments, the silica core has a diameter less than 10 nm (e.g., less than 9 nm, e.g., less than 8 nm, e.g., less than 7 nm, e.g., less than 6 nm, e.g., within a range from 2.7 nm to 5.8 nm).

In some embodiments, each of the nanoparticles comprises a polyethylene glycol (PEG) shell (e.g., a partial coating or complete coating). In some embodiments, the thickness of the PEG shell is less than 2 nm (e.g., about 1 nm).

In some embodiments, the nanoparticle comprises a chelator. In some embodiments, the chelator is selected from the group comprising 1,4,7,10-tetraazacyclododecane-1,4,7,10-tetraacetic acid (DOTA), DOTA-Bz-SCN, and desferoxamine (DFO).

In some embodiments, local concentration of nanoparticles within a microenvironment of a tumor of the subject is in a range from about 0.013 nmol/cm³ to about 86 nmol/cm³ or from about 0.013 nmol/cm³ to about 0.14 nmol/cm3 or from about 8 nmol/cm³ to about 86 nmol/cm³ (e.g., greater than or equal to about 2 μM, greater than or equal to about 3 μM, greater than or equal to about 5 μM, greater than or equal to about 8 μM, greater than or equal to about 10 μM, or greater than or equal to about 15 μM).

In some embodiments, an administered dose (e.g., by IV administration) has a particle concentration from about 100 nM to about 60 μM, or wherein an administered dose has particle concentration less than 150 nM. (e.g., less than 100 nM, e.g., less than 50 nM, less than 10 nM, less than 5 nM)).

In some embodiments, an administered dose has particle concentration greater than or equal to about 1 μM.

In another aspect, the invention is directed to a composition for use in the method of any one of the preceding claims, the composition comprising ultrasmall nanoparticles having the following attributes: (i) a number of targeting ligands from 5 to 60 per nanoparticle; (ii) a heterogeneous surface characterized by one or more of (a) to (d) as follows: (a) an unincorporated dye; (b) a variation in a PEG coating; (c) a variation in dye encapsulation; and (d) a variation in number of targeting ligands; (iii) a particle core and shell having a hydrodynamic diameter in a range from 4.7 nm to 7.8 nm (e.g., with a silica core diameter in a range from 2.7 nm to 5.8 nm and/or with a PEG shell thickness of about 1 nm); and (iv) a silica composition controlled for ferroptosis.

In some embodiments, the silica composition controlled for ferroptosis comprises nanoparticles made using a ratio of phosphonate-silane to tetramethyl orthosilicate (TMOS) in a reaction feed at or above 20% such that ferroptosis may occur. In some embodiments, the silica composition controlled for ferroptosis comprises nanoparticles made using a ratio of phosphonate-silane to tetramethyl orthosilicate (TMOS) in a reaction feed from 0% to 20% such that ferroptosis may not occur.

In some embodiments, the polyethylene glycol (PEG) coating comprises from about 100 to about 500 PEG chains per nanoparticle. In some embodiments, the dye encapsulation is by PEG. In some embodiments, the targeting ligands range from 1 to 60 per nanoparticle, or from 1 to 15 per nanoparticle, or from 40 to 60 per nanoparticle.

In another aspect, the invention is directed to a method of identifying a treatment for a subject or group of subjects involving administration of silica nanoparticles (e.g., to enhance an immunotherapy), said method comprising identifying one or more of biomarkers (e.g., via immunophenotyping for characterizing an immune response).

In some embodiments, the one or more biomarkers comprise one or more of the following:

Markers for Immunophenotyping

| Marker | Cell Type |
|---|---|
| Th1 cells | CD3+, CD4+, IFN-γ+, Tbet+ CXCR3+ |
| Th2 cells | CD3+, CD4+, GATA-3+, CCR4+ |
| Activated CD8+ | CD3+, CD8+, CXCR3+ |
| T cells | |
| Tregs | CD3+, CD4+. CD25+, FoxP3+ |
| T helpers | CD3+, CD4+, FoxP3− |
| Inhibitory T cells | CD8/CD4 (LAG3+, TIM-3+, PD1+, CTLA4+) |
| M-MDSC | CD11b+, Ly6G−, LY6Chigh |
| G-MDSC | CD11b+, Ly6G+, LY6Clow |
| Macrophage M1 | CD11b+, MHCII+, iNOS |
| Macrophage M2 | CD11b+, MCHII+, CD206+, Arginase1+ |
| Dendritic cells | CD11b+, CD11c+, MHCII+ (CD40, CD80, CD86) |
| (DCs) | |
| Monocytes | CD11b+, LY6G+, MGCII+ |
| Neutrophils | Ly6G+/− |
| Natural Killer (NK) | NK1.1 |
| cells | |

In another aspect, the invention is directed to a method of identifying a treatment for a subject or group of subjects involving administration of silica nanoparticles (e.g., to enhance an immunotherapy), said method comprising identifying one or more biomarkers.

In some embodiments, the one or more biomarkers comprise one or more of the following:

Markers for Adaptive Immune Response

| Gene | Gene | | |
|---|---|---|---|
| Number | Symbol | Gene Name | Annotation |
| 1 | Ccl20 | Chemokine (C-C | Binds to CCR6. Recruits DCs, effector/memory |
| | | motif) ligand 20 | T-cells and B-cells, Th17 and Treg cells. |
| 2 | Ccl22 | Chemokine (C-C | Binds to CCR4. In OC, TAMs secrete CCL22 to |
| | | motif) ligand 22 | attract Treg cells. (PMID: 28555670). |
| 3 | Cd401g | CD40 ligand | Binds to CD40. Upon binding to CD40 (CD138) |
| | | | on DCs, CD40L mediates CD8+ T-cell immunity |
| | | | via secretion of IL-12 and IFN-γ. Some CAR-T |
| | | | cells are designed to express CD40L (PMID: |
| | | | 27068948). |
| 4 | Csf1 | Colony stimulating | Plays an essential role in the regulation of |
| | | factor 1 (macrophage) | survival, proliferation and differentiation of |
| | | | macrophages and monocytes. Blocking |
| | | | Csf1/Csf1R signaling in the TME decreases |
| | | | TAM (PMID: 25082815). |
| 5 | Cxcl10 | Chemokine (C-X-C | Ligand for CXCR3. Increased CXCL10 is |
| | | motif) ligand 10 | associated with increased CD8 T infiltrating |
| | | | tumor cells. Elevated serum levels of CXCL9 and |
| | | | 10 are associated with higher survival rates in |
| | | | patients with OC (PMID: 27490802). |
| 6 | Cxcl12 | Chemokine (C-X-C | Ligand for CXCR4. Recruits Treg cells. (PMID: |
| | | motif) ligand 12 | 26629936) |
| 7 | Cxcl5 | Chemokine (C-X-C | Ligand for CXCR2. Involved in neutrophil |
| | | motif) ligand 5 | activation. Associated with late stage gastric |
| | | | cancer (PMID: 17479287) and poor survival in |
| | | | pancreatic cancer (PMID: 21356384). Recruits |
| | | | MDSC (PMID: 28555670). |
| 8 | Cxcl9 | Chemokine (C-X-C | Ligand for CXCR3. CD8 T cells, Th1 and NK |
| | | motif) ligand 9 | cells express CXCR3. Increased CXCL10 is |
| | | | associated with increased CD8 T infiltrating |
| | | | tumor cells. Elevated serum levels of CXCL9 and |
| | | | 10 are associated with higher survival rates in |
| | | | patients with ovarian cancer (PMID: 27490802). |
| 9 | Fasl | Fas ligand (TNF | Involved in cytotoxic T-cell-mediated apoptosis, |
| | | superfamily; | natural killer cell-mediated apoptosis and in T- |
| | | member 6) | cell development (PMID: 9228058, PMID: |
| | | | 7528780, PMID: 9427603). High levels of FasL |
| | | | in the tumor stroma decrease CD8 T cell |
| | | | infiltration in tumors (PMID: 24793239) and help |
| | | | tumor escape immune attack (PMID: 17667919). |
| 10 | Ifng | Interferon gamma | Secreted by Th1 and Type I NKT cells. Activates |
| | | | CD8 T cells and DCs. |
| 11 | Ifna1 | Interferon alpha-1 | Produced by macrophages, IFN-alpha have |
| | | | antiviral activities. |
| 12 | Il10 | Interleukin 10 | Ligand for the heterotetrameric receptor |
| | | | comprised of IL10RA and IL10RB. Targets |
| | | | macrophages and monocytes and limits their |
| | | | release of pro-inflammatory cytokines including |
| | | | GM-CSF, IL-1 alpha, IL-1 beta, IL-6, IL-8 and |
| | | | TNF-alpha (PMID: 1940799, PMID: 7512027, |
| | | | PMID: 11564774). Interferes with antigen |
| | | | presentation by reducing the expression of MHC- |
| | | | class II and co-stimulatory molecules, thereby |
| | | | inhibiting their ability to induce T cell |
| | | | activation (PMID: 8144879). |
| 13 | Il12b | Interleukin 12B | Acts as a growth factor for activated T and NK |
| | | | cells, enhance the lytic activity of |
| | | | NK/lymphokine-activated killer cells, and |
| | | | stimulate the production of IFN-γ. |
| 14 | Il13 | Interleukin 13 | Ligand for IL-13Rα1 and IL-13Rα2. Interacts |
| | | | with IL4 to mediate tumor progression but |
| | | | synergizes with IL2 to increase production of |
| | | | IFN-γ (PMID: 8096327). |
| 15 | Il15 | Interleukin 15 | Activates T cells and NK cells. IL15 agonist |
| | | | ALT803 enhances NK function against ovarian |
| | | | cancer (PMID: 30410679, 28236454). |
| 16 | Il17a | Interleukin 17A | Ligand for IL 17RA and IL 17RC. Induces stromal |
| | | | cells to produce proinflammatory and |
| | | | hematopoietic cytokines (PMID: 8676080). |
| | | | Stimulates CD133+ cells in OC and drives tumor |
| | | | progression (PMID: 24362529). |
| 17 | Il1a | Interleukin 1 alpha | IL1A is a potential diagnostic biomarker for |
| | | | NSCLC (PMID: 25554695). |
| 18 | Il1b | Interleukin 1 beta | Promotes Th17 differentiation of T-cells. |
| 19 | Il2 | Interleukin 2 | Produced by T-cells in response to antigenic or |
| | | | mitogenic stimulation, it is required for T-cell |
| | | | proliferation. |
| 20 | Il23a | Interleukin 23, alpha | Promotes inflammatory responses and increases |
| | | subunit p19 | angiogenesis. Necessary for activation of Th17 |
| | | | cells. |
| 21 | Il4 | Interleukin 4 | Immunosuppressive cytokine in the TME |
| | | | (PMID: 28733709). Activates Th2 cells. |
| 22 | Il6 | Interleukin 6 | Increased levels of IL6 are associated with |
| | | | poor prognosis in patients (PMID: 21795409). |
| | | | Involved in lymphocyte and monocyte |
| | | | differentiation. Required for the generation |
| | | | of Th17 and Treg cells. |
| 23 | Il7 | Interleukin 7 | Decreased Treg tumor infiltration and apoptosis |
| | | | of T cells (PMID: 19454692). |
| 24 | Il9 | Interleukin 9 | Supports IL-2 and IL-4 independent growth of |
| | | | helper T-cells. (PMID: 27832300). |
| 25 | Inha | Inhibin alpha | Activates T cells. |
| 26 | Nfkb1 | Nuclear factor of kappa | Elevated expression associated with poor |
| | | light polypeptide gene | survival in newly diagnosed patients in OC |
| | | enhancer in B-cells 1, | (PMID: 20564628 - 2010). |
| | | p105 | |
| 27 | Nos2 | Nitric oxide synthase 2, | M1 macrophage marker. |
| | | inducible | |
| 28 | Tnf | Tumor necrosis factor- | In the TME increases myeloid cell recruitment in |
| | | alpha | an IL-17-dependent manner that contributes to |
| | | | tumor growth (PMID: 19741298). |
| 29 | Tgfb1 | TGF-Beta 1 | Can promote Th17 or Treg lineage differentiation |
| | | | by expression of Foxp3 (PMID: 14676299 - 2003). |
| 30 | Prf1 | Perforin 1 | Cytolytic protein produced by T and NK cells. |
| 31 | Gzma | Granzyme A | Cytolytic protein produced by T and NK cells. |

Markers for Innate Immune Response, Antigen Presentation and DAMPs

| Gene | Gene | | |
|---|---|---|---|
| Number | Symbol | Gene Name | Annotation |
| 32 | H2-D1 | MHC Ia, Histocompatibility | Involved in the presentation of foreign |
| | | 2, D1 | antigens to the immune system. |
| 33 | H2-T23 | MHC Ib, H-2 class I | Involved in the presentation of foreign |
| | | histocompatibility antigen, | antigens to the immune system. |
| | | D-37 alpha chain | |
| 34 | B2M | Beta-2-microglobulin | Component of the class I major |
| | | | histocompatibility complex (MHC). |
| | | | Involved in Ag presentation to the immune |
| | | | system. |
| 33 | TAP1 | Antigen peptide transporter 1 | Involved in the transport of antigens from |
| | | | the cytoplasm to the endoplasmic reticulum |
| | | | for association with MHC class I molecules. |
| 34 | TAP2 | Antigen peptide transporter 2 | Involved in the transport of antigens from |
| | | | the cytoplasm to the endoplasmic reticulum |
| | | | for association with MHC class I molecules. |
| 35 | Calr | Calreticulin | Translocation to the extracellular space is an |
| | | | early indicator of cell stress response |
| | | | (PMID: 18573340). |
| 36 | Hspa1b | Heat shock 70 kDa protein 1B | Early indicator of cell stress response |
| | | | (PMID: 18573340). |
| 37 | Hsp90ab1 | Heat shock protein HSP 90- | Early indicator of cell stress response |
| | | beta | (PMID: 18573340). |
| 38 | IFNB1 | Interferon Beta I | Indicator of innate immune response. |
| 39 | IFNA1 | Interferon Alpha I | Indicator of innate immune response. |
| 40 | IFI204 | Interferon-activable protein | Essential for IRF3 and NF-kB activation |
| | | 204 | and induction of IFN beta 1. |
| 41 | IFI44 | Interferon-induced protein 44 | Upregulated in OC (PMID: 17145569). |
| 42 | HMGB1 | High mobility group box 1 protein | DAMPs marker |
| 43 | S100A8 | S100 calcium-binding protein A8 | DAMPs marker |
| 44 | S100A9 | S100 calcium-binding protein A9 | DAMPs marker |
| 45 | STING | Stimulator of interferon genes | DAMPs marker |

Markers for Ferroptosis, Iron metabolism and Antioxidant

| Gene | Gene | | |
|---|---|---|---|
| Number | Symbol | Gene Name | Annotation |
| 46 | Fth1 | Ferritin Heavy Chain | Stores iron in a soluble, non-toxic, readily |
| | | | available form. |
| 47 | Aco1 | Aconitase 1 | When cellular iron levels are high, the |
| | | | encoded protein functions as an aconitase, |
| | | | an essential enzyme in the TCA cycle that |
| | | | catalyzes the conversion of citrate to |
| | | | isocitrate. When cellular iron levels are low, |
| | | | the encoded protein regulates iron uptake |
| | | | and utilization by binding to iron-responsive |
| | | | elements in the untranslated regions of mRNAs |
| | | | for genes involved in iron metabolism. |
| 48 | Tfrc | Transferrin receptor | Iron uptake via receptor-mediated endocytosis. |
| 49 | Slc40a1 | Ferroportin-1 | Iron exporter. |
| 50 | Alas2 | Aminolevulinic acid synthase | Locates in the mitochondria. Maintains iron |
| | | 2, erythroid | homeostasis. |
| 51 | Slc3a2 | 4F2 cell-surface antigen heavy | (IFNγ) released from CD8+ T cells |
| | | chain | downregulates the expression of SLC3A2 |
| | | | and SLC7A11, two subunits of the |
| | | | glutamate-cystine antiporter system xc−, |
| | | | impairs the uptake of cystine by tumour |
| | | | cells, and as a consequence, promotes |
| | | | tumour cell lipid peroxidation and |
| | | | ferroptosis. (PMID: 31043744) |
| 52 | Slc7a11 | Cystine/glutamate transporter | (IFNγ) released from CD8+ T cells |
| | | | downregulates the expression of SLC3A2 |
| | | | and SLC7A11, two subunits of the |
| | | | glutamate-cystine antiporter system xc−, |
| | | | impairs the uptake of cystine by tumour |
| | | | cells, and as a consequence, promotes |
| | | | tumour cell lipid peroxidation and |
| | | | ferroptosis. (PMID: 31043744) |
| 53 | Acsl4 | Long-chain-fatty-acid--CoA | Cells resistant to ferroptosis exhibit reduced |
| | | ligase 4 | levels of Acsl4. |
| 54 | Gpx4 | Glutathione peroxidase 4 | Protects cells against membrane lipid |
| | | | peroxidation. |
| 55 | Nfe2l2 | NRF2 | Transcription factor controlling antioxidant |
| | | | genes including GCLC and NQO1. |
| 56 | Nqo1 | NAD(P)H quinone | Antioxidant enzyme regulated by NRF2. |
| | | dehydrogenase 1 | |
| 57 | Sod2 | Superoxide dismutase 2, | Transforms toxic superoxide into hydrogen |
| | | mitochondrial | peroxide. |
| 58 | Gclc | Glutamate-cysteine ligase | First enzyme in the glutathione (GSH) |
| | | catalytic subunit | biosynthesis pathway. |
| 59 | Gss | Glutathione synthetase | Second enzyme in the glutathione (GSH) |
| | | | biosynthesis pathway. |

In some embodiments, the method is performed via real-time quantitative reverse transcription polymerase chain reaction (qRT-PCR) (e.g., for characterizing an immune response). In some embodiments, the method comprises identifying one or more pattern recognition receptors (e.g., STING (Stimulator of interferon genes), TLR (Toll-like receptor), RIG-I (Retinoic acid-inducible gene I) biomarkers.

In another aspect, the invention is directed to a kit comprising a nanoparticle composition in a unit dosage effective for enhanced treatment of cancer in a subject having been diagnosed with the cancer and receiving (or having received) therapy engineered cellular therapies (e.g., chimeric antigen receptor (CAR) T-cells), the nanoparticle composition comprising ultrasmall nanoparticles.

In some embodiments, the nanoparticles comprise tumor-targeting ligands. In some embodiments, the nanoparticles do not comprise tumor-targeting ligands. In some embodiments, the composition of the nanoparticle (i) augments intratumoral immune responsiveness and cytotoxicity and/or (ii) improves CAR T cell exhaustion and/or persistence.

In another aspect, the invention is directed to a kit comprising a nanoparticle composition in a unit dosage effective for enhanced treatment of cancer (e.g., one or more tumors having a high level of myeloid cells, a main driver of immune evasion e.g., melanoma or triple negative breast cancer, TNBC) in a subject having been diagnosed with the cancer and receiving (or having received) therapy with a myeloid-targeting inhibitor and one or more immune checkpoint blockade antibodies, the nanoparticle composition comprising ultrasmall nanoparticles, said nanoparticles comprising targeting ligands.

In some embodiments, the cancer comprises one or more tumors comprised of high levels of myeloid cells. In some embodiments, the myeloid-targeting inhibitor comprises a PI3Kγ-selective inhibitor targeting myeloid cells (e.g., IPI-549). In some embodiments, the one or more immune checkpoint blockade antibodies comprises a member selected from the group consisting of ipilimumab (anti-CTLA4), pembrolizumab (anti-PD1), nivolumab (anti-PD1), and atezolizumab (anti-PD-L1). In some embodiments, the targeting ligands comprise melanocortin-1 receptor (MC1-R) targeting ligands of a single type or multiple type. In some embodiments, resistance to immune ICB is limited by combining particle-driven cytotoxic processes (e.g., ferroptosis, immune-related cell death) and enhanced pro-inflammatory responses with one or more immune checkpoint blockade antibodies and/or selective PI3Kγ-targeting to subvert immunosuppressive components in a tumor microenvironment (TME).

In another aspect, the invention is directed to a kit comprising (a) a nanoparticle composition in a unit dosage effective for enhanced treatment of cancer (e.g., prostate cancer) in a subject having been diagnosed with the cancer and receiving (or having received) therapy with one or more immune checkpoint blockade antibodies, the nanoparticle composition comprising ultrasmall nanoparticles, said nanoparticles comprising targeting ligands; and (b) a molecular radiotherapeutic, e.g., a composition comprising peptide radioligands (e.g., radiolabeled PSMA-targeting ligands) (e.g., further comprising a myeloid-targeting inhibitor (e.g., a PI3Kγ-selective inhibitor targeting myeloid cells (e.g., IPI-549))).

In some embodiments, molecular radiotherapy comprise a radiotherapeutic label. In some embodiments, the radiotherapeutic label comprises an alpha-emitting radioisotope or a beta-emitting radioisotope. In some embodiments, the alpha-emitting radioisotope comprises ²²⁵Ac. In some embodiments, the beta-emitting radioisotope comprises ¹⁷⁷Lu.

In some embodiments, the one or more immune checkpoint blockade antibodies comprises a member selected from the group consisting of ipilimumab (anti-CTLA4), pembrolizumab (anti-PD1), nivolumab (anti-PD1), and atezolizumab (anti-PD-L1).

In some embodiments, targeting ligands comprise PSMA-targeting ligands.

In some embodiments, the nanoparticles have a diameter no greater than 20 nm. In some embodiments, the nanoparticles have a diameter no greater than 10 nm. In some embodiments, the nanoparticles comprise silica. In some embodiments, each of the nanoparticles comprises 1 to 25 targeting ligands (e.g., of a single species) (e.g., 2 to 20 ligands, 5 to 15 ligands, 5 to 10 ligands, or about 6-8 ligands).

In some embodiments, the cancer comprises prostate cancer, ovarian cancer (e.g., high-grade ovarian cancer), malignant brain tumors, melanoma, breast cancer, or lung cancer.

In another aspect, the invention is directed to a method comprising administering to a subject a nanoparticle composition in a unit dosage effective for enhanced treatment of cancer (e.g., ovarian cancer), wherein the subject has been diagnosed with the cancer and is receiving (or has received) therapy with cells (e.g., T cells, e.g., dendritic cells, e.g., engineered cells, e.g., chimeric antigen receptor (CAR) T-cells), the nanoparticle composition comprising ultrasmall nanoparticles.

In some embodiments, the nanoparticles comprise tumor-targeting ligands. In some embodiments, the nanoparticles do not comprise tumor-targeting ligands. In some embodiments, the nanoparticle composition (i) augments intratumoral immune responsiveness and cytotoxicity and/or (ii) improves CAR T cell exhaustion.

In another aspect, the invention is directed to a method comprising administering to a subject a nanoparticle composition in a unit dosage effective for enhanced treatment of cancer (e.g., one or more tumors having a high level of myeloid cells, a main driver of immune evasion e.g., melanoma or triple negative breast cancer, TNBC), wherein the subject has been diagnosed with the cancer and is receiving (or has received) therapy with a myeloid-targeting inhibitor and one or more immune checkpoint blockade antibodies, the nanoparticle composition comprising ultrasmall nanoparticles, said nanoparticles comprising targeting ligands.

In some embodiments, the cancer comprises one or more tumors having a high level of myeloid cells. In some embodiments, the myeloid-targeting inhibitor comprises a PI3Kγ-selective inhibitor targeting myeloid cells (e.g., IPI-549). In some embodiments, the one or more immune checkpoint blockade antibodies comprises a member selected from the group consisting of ipilimumab (anti-CTLA4), pembrolizumab (anti-PD1), nivolumab (anti-PD1), and atezolizumab (anti-PD-L1). In some embodiments, the targeting ligands comprise melanocortin-1 receptor (MC1-R) targeting ligands of a single type or multiple types. In some embodiments, resistance to immune ICB is limited by combining particle-driven ferroptosis and enhanced pro-inflammatory responses with one or more immune checkpoint blockade antibodies and/or selective PI3Kγ-targeting to subvert immunosuppressive components in a tumor microenvironment (TME).

In another aspect, the invention is directed to a method comprising administering to a subject (a) a nanoparticle composition in a unit dosage effective for enhanced treatment of cancer (e.g., prostate cancer), wherein the subject has been diagnosed with the cancer and is receiving (or has received) therapy with one or more immune checkpoint blockade antibodies, the nanoparticle composition comprising ultrasmall nanoparticles, said nanoparticles comprising targeting ligands; and (b) external beam radiotherapy (or molecular radiotherapy, e.g., peptide radioligands (e.g., radiolabeled PSMA-targeting ligands)).

In some embodiments, the molecular radiotherapy comprise a radiotherapeutic label. In some embodiments, the radiotherapeutic label comprises an alpha-emitting radioisotope or a beta-emitting radioisotope. In some embodiments, the alpha-emitting radioisotope comprises ²²Ac. In some embodiments, the beta-emitting radioisotope comprises ¹⁷⁷Lu. In some embodiments, the one or more immune checkpoint blockade antibodies comprises a member selected from the group consisting of ipilimumab (anti-CTLA4), pembrolizumab (anti-PD1), nivolumab (anti-PD1), and atezolizumab (anti-PD-L1). In some embodiments, the targeting ligands comprise PSMA-targeting ligands of a single type or multiple types.

In some embodiments, the method further comprises administering to the subject a myeloid-targeting inhibitor. In some embodiments, the myeloid-targeting inhibitor comprises a PI3Kγ-selective inhibitor targeting myeloid cells (e.g., IPI-549).

In some embodiments, the nanoparticles have a diameter no greater than 20 nm. In some embodiments, the nanoparticles have a diameter no greater than 10 nm. In some embodiments, the nanoparticles comprise silica. In some embodiments, each of the nanoparticles comprises 1 to 25 targeting ligands (e.g., of a single species) (e.g., 2 to 20 ligands, 5 to 15 ligands, 5 to 10 ligands, or about 6-8 ligands).

In some embodiments, the cancer comprises prostate cancer, ovarian cancer (e.g., high-grade ovarian cancer), malignant brain tumors, or melanoma.

Elements of embodiments involving one aspect of the invention (e.g., methods) can be applied in embodiments involving other aspects of the invention (e.g., systems), and vice versa.

### Definitions

In order for the present disclosure to be more readily understood, certain terms are first defined below. Additional definition for the following terms and other terms are set forth throughout the specification.

Administration: As used herein, the term “administration” typically refers to the administration of a composition comprising a nanoparticle to a subject or system. In general, any route of administration may be utilized including, for example, parenteral (e.g., intravenous), oral, topical, subcutaneous, peritoneal, intraarterial, inhalation, vaginal, rectal, nasal, introduction into the cerebrospinal fluid, or instillation into body compartments. In certain embodiments, administration is oral. Additionally or alternatively, in certain embodiments, administration is parenteral. In certain embodiments, administration is intravenous. In certain embodiments, administration is intraperitoneal.

Agent: The term “agent”, as used herein, may refer to a compound, molecule, or entity of any chemical and/or biological class including, for example, a small molecule, polypeptide, nucleic acid, saccharide, lipid, metal, or a combination or complex thereof. In certain embodiments, the term “agent” may refer to a compound, molecule, or entity that comprises a polymer. In certain embodiments, the term may refer to a compound or entity that comprises one or more polymeric moieties. In certain embodiments, the term may refer to a compound, molecule, or entity that lacks or is substantially free of any polymer or polymeric moiety. In some embodiments, the term may refer to a nanoparticle.

Antibody: As used herein, the term “antibody” refers to a polypeptide that includes canonical immunoglobulin sequence elements sufficient to confer specific binding to a particular target antigen. As is known in the art, intact antibodies as produced in nature are approximately 150 kD tetrameric agents comprised of two identical heavy chain polypeptides (about 50 kD each) and two identical light chain polypeptides (about 25 kD each) that associate with each other into what is commonly referred to as a “Y-shaped” structure. Each heavy chain is comprised of at least four domains (each about 110 amino acids long)-an amino-terminal variable (VH) domain (located at the tips of the Y structure), followed by three constant domains: CH1, CH2, and the carboxy-terminal CH3 (located at the base of the Y's stem). A short region, known as the “switch”, connects the heavy chain variable and constant regions. The “hinge” connects CH2 and CH3 domains to the rest of the antibody. Two disulfide bonds in this hinge region connect the two heavy chain polypeptides to one another in an intact antibody. Each light chain is comprised of two domains—an amino-terminal variable (VL) domain, followed by a carboxy-terminal constant (CL) domain, separated from one another by another “switch”. Intact antibody tetramers are comprised of two heavy chain-light chain dimers in which the heavy and light chains are linked to one another by a single disulfide bond; two other disulfide bonds connect the heavy chain hinge regions to one another, so that the dimers are connected to one another and the tetramer is formed. Naturally-produced antibodies are also glycosylated, typically on the CH2 domain. Each domain in a natural antibody has a structure characterized by an “immunoglobulin fold” formed from two beta sheets (e.g., 3-, 4-, or 5-stranded sheets) packed against each other in a compressed antiparallel beta barrel. Each variable domain contains three hypervariable loops known as “complement determining regions” (CDR1, CDR2, and CDR3) and four somewhat invariant “framework” regions (FR1, FR2, FR3, and FR4). When natural antibodies fold, the FR regions form the beta sheets that provide the structural framework for the domains, and the CDR loop regions from both the heavy and light chains are brought together in three-dimensional space so that they create a single hypervariable antigen binding site located at the tip of the Y structure. The Fc region of naturally-occurring antibodies binds to elements of the complement system, and also to receptors on effector cells, including for example effector cells that mediate cytotoxicity. As is known in the art, affinity and/or other binding attributes of Fc regions for Fc receptors can be modulated through glycosylation or other modification. In some embodiments, antibodies produced and/or utilized in accordance with the present invention include glycosylated Fc domains, including Fc domains with modified or engineered such glycosylation. For purposes of the present disclosure, in certain embodiments, any polypeptide or complex of polypeptides that includes sufficient immunoglobulin domain sequences as found in natural antibodies can be referred to and/or used as an “antibody”, whether such polypeptide is naturally produced (e.g., generated by an organism reacting to an antigen), or produced by recombinant engineering, chemical synthesis, or other artificial system or methodology. In some embodiments, an antibody is polyclonal; in some embodiments, an antibody is monoclonal. In some embodiments, an antibody has constant region sequences that are characteristic of mouse, rabbit, primate, or human antibodies. In some embodiments, antibody sequence elements are humanized, primatized, chimeric, etc., as is known in the art. Moreover, the term “antibody” as used herein, can refer in appropriate embodiments (unless otherwise stated or clear from context) to any of the art-known or developed constructs or formats for utilizing antibody structural and functional features in alternative presentation. For example, an antibody utilized in accordance with certain embodiments of the present invention is in a format selected from, but not limited to, intact IgA, IgG, IgE or IgM antibodies; bi- or multi-specific antibodies (e.g., Zybodies®, etc); antibody fragments such as Fab fragments, Fab′ fragments, F(ab′)2 fragments, Fd′ fragments, Fd fragments, and isolated CDRs or sets thereof, single chain Fvs; polypeptide-Fc fusions; single domain antibodies (e.g., shark single domain antibodies such as IgNAR or fragments thereof); cameloid antibodies; masked antibodies (e.g., Probodies®); Small Modular ImmunoPharmaceuticals (“SMIPs™”); single chain or Tandem diabodies (TandAb®); VHHs; Anticalins®; Nanobodies® minibodies; BiTE®s; ankyrin repeat proteins or DARPINs®; Avimers®; DARTs; TCR-like antibodies, Adnectins®; Affilins®; Trans-bodies®; Affibodies®; TrimerX®; MicroProteins; Fynomers®, Centyrins®; and KALBITOR®s.

Antibody agent: As used herein, the term “antibody agent” refers to an agent that specifically binds to a particular antigen. In some embodiments, the term encompasses any polypeptide or polypeptide complex that includes immunoglobulin structural elements sufficient to confer specific binding. Exemplary antibody agents include, but are not limited to monoclonal antibodies or polyclonal antibodies. In some embodiments, an antibody agent may include one or more constant region sequences that are characteristic of mouse, rabbit, primate, or human antibodies. In some embodiments, an antibody agent may include one or more sequence elements are humanized, primatized, chimeric, etc, as is known in the art. In many embodiments, the term “antibody agent” is used to refer to one or more of the art-known or developed constructs or formats for utilizing antibody structural and functional features in alternative presentation. For example, embodiments, an antibody agent utilized in accordance with certain embodiments of the present invention is in a format selected from, but not limited to, intact IgA, IgG, IgE or IgM antibodies; bi- or multi-specific antibodies (e.g., Zybodies®, etc); antibody fragments such as Fab fragments, Fab′ fragments, F(ab′)2 fragments, Fd′ fragments, Fd fragments, and isolated CDRs or sets thereof; single chain Fvs; polypeptide-Fc fusions; single domain antibodies (e.g., shark single domain antibodies such as IgNAR or fragments thereof); cameloid antibodies; masked antibodies (e.g., Probodies®); Small Modular ImmunoPharmaceuticals (“SMIPs™”); single chain or Tandem diabodies (TandAb®); VHHs; Anticalins®; Nanobodies® minibodies; BiTE®s; ankyrin repeat proteins or DARPINs®; Avimers®; DARTs; TCR-like antibodies, Adnectins®; Affilins®; Trans-bodies®; Affibodies®; TrimerX®; MicroProteins; Fynomers®, Centyrins®; and KALBITOR®s. In some embodiments, an antibody may lack a covalent modification (e.g., attachment of a glycan) that it would have if produced naturally. In some embodiments, an antibody may contain a covalent modification (e.g., attachment of a glycan, a payload or other pendant group). In many embodiments, an antibody agent is or comprises a polypeptide whose amino acid sequence includes one or more structural elements recognized by those skilled in the art as a complementarity determining region (CDR); in some embodiments an antibody agent is or comprises a polypeptide whose amino acid sequence includes at least one CDR (e.g., at least one heavy chain CDR and/or at least one light chain CDR) that is substantially identical to one found in a reference antibody. In some embodiments an included CDR is substantially identical to a reference CDR in that it is either identical in sequence or contains between 1-5 amino acid substitutions as compared with the reference CDR. In some embodiments an included CDR is substantially identical to a reference CDR in that it shows at least 85%, 86%, 87%, 88%, 89%, 90%, 91%, 92%, 93%, 94%, 95%, 96%, 97%, 98%, 99%, or 100% sequence identity with the reference CDR. In some embodiments an included CDR is substantially identical to a reference CDR in that it shows at least 96%, 96%, 97%, 98%, 99%, or 100% sequence identity with the reference CDR. In some embodiments an included CDR is substantially identical to a reference CDR in that at least one amino acid within the included CDR is deleted, added, or substituted as compared with the reference CDR but the included CDR has an amino acid sequence that is otherwise identical with that of the reference CDR. In some embodiments an included CDR is substantially identical to a reference CDR in that 1-5 amino acids within the included CDR are deleted, added, or substituted as compared with the reference CDR but the included CDR has an amino acid sequence that is otherwise identical to the reference CDR. In some embodiments an included CDR is substantially identical to a reference CDR in that at least one amino acid within the included CDR is substituted as compared with the reference CDR but the included CDR has an amino acid sequence that is otherwise identical with that of the reference CDR. In some embodiments an included CDR is substantially identical to a reference CDR in that 1-5 amino acids within the included CDR are deleted, added, or substituted as compared with the reference CDR but the included CDR has an amino acid sequence that is otherwise identical to the reference CDR. In some embodiments, an antibody agent is or comprises a polypeptide whose amino acid sequence includes structural elements recognized by those skilled in the art as an immunoglobulin variable domain. In some embodiments, an antibody agent is a polypeptide protein having a binding domain which is homologous or largely homologous to an immunoglobulin-binding domain.

Antigen: The term “antigen”, as used herein, refers to an agent that elicits an immune response; and/or (ii) an agent that binds to a T cell receptor (e.g., when presented by an MHC molecule) or to an antibody. In some embodiments, an antigen elicits a humoral response (e.g., including production of antigen-specific antibodies); in some embodiments, an elicits a cellular response (e.g., involving T-cells whose receptors specifically interact with the antigen). In some embodiments, an antigen binds to an antibody and may or may not induce a particular physiological response in an organism.

Antigen presenting cell: The phrase “antigen presenting cell” or “APC,” as used herein, has its art understood meaning referring to cells which process and present antigens to T-cells. Exemplary antigen cells include dendritic cells, macrophages and certain activated epithelial cells.

Biocompatible: The term “biocompatible”, as used herein, refers to materials that do not cause significant harm to living tissue when placed in contact with such tissue, e.g., in vivo. In certain embodiments, materials are “biocompatible” if they are not toxic to cells. In certain embodiments, materials are “biocompatible” if their addition to cells in vitro results in less than or equal to 20% cell death. In certain embodiments, materials are biodegradable.

Cancer: As used herein, the term “cancer” refers to a malignant neoplasm or tumor (Stedman's Medical Dictionary, 25th ed.; Hensly ed.; Williams & Wilkins: Philadelphia, 1990). Exemplary cancers include, but are not limited to, acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; eye cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematopoietic cancers (e.g., leukemia such as acute lymphocytic leukemia (ALL) (e.g., B cell ALL, T cell ALL), acute myelocytic leukemia (AML) (e.g., B cell AML, T cell AML), chronic myelocytic leukemia (CML) (e.g., B cell CML, T cell CML), and chronic lymphocytic leukemia (CLL) (e.g., B cell CLL, T cell CLL)); lymphoma such as Hodgkin lymphoma (HL) (e.g., B cell HL, T cell HL) and non-Hodgkin lymphoma (NHL) (e.g., B cell NHL such as diffuse large cell lymphoma (DLCL) (e.g., diffuse large B cell lymphoma), follicular lymphoma, chronic lymphocytic leukemia/small lymphocytic lymphoma (CLL/SLL), mantle cell lymphoma (MCL), marginal zone B cell lymphomas (e.g., mucosa associated lymphoid tissue (MALT) lymphomas, nodal marginal zone B cell lymphoma, splenic marginal zone B cell lymphoma), primary mediastinal B cell lymphoma, Burkitt lymphoma, lymphoplasmacytic lymphoma (e.g., Waldenstrom's macroglobulinemia), hairy cell leukemia (HCL), immunoblastic large cell lymphoma, precursor B lymphoblastic lymphoma and primary central nervous system (CNS) lymphoma; and T cell NHL such as precursor T lymphoblastic lymphoma/leukemia, peripheral T cell lymphoma (PTCL) (e.g., cutaneous T cell lymphoma (CTCL) (e.g., mycosis fungoides, Sezary syndrome), angioimmunoblastic T cell lymphoma, extranodal natural killer T cell lymphoma, enteropathy type T cell lymphoma, subcutaneous panniculitis like T cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES); neuroblastoma; neurofibroma (e.g., neurofibromatosis (NF) type 1 or type 2, schwannomatosis); neuroendocrine cancer (e.g., gastroenteropancreatic neuroendocrine tumor (GEP NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic adenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva).

Chemotherapeutic Agent: As used herein, the term “chemotherapeutic agent” or “oncolytic therapeutic agent” (e.g., anti-cancer drug, e.g., anti-cancer therapy, e.g., immune cell therapy) has its art-understood meaning referring to one or more pro-apoptotic, cytostatic and/or cytotoxic agents, and/or hormonal agents, for example, specifically including agents utilized and/or recommended for use in treating one or more diseases, disorders or conditions associated with undesirable cell proliferation. In many embodiments, chemotherapeutic agents and/or oncolytic therapeutic agents are useful in the treatment of cancer. In some embodiments, a chemotherapeutic agent and/or oncolytic therapeutic agents may be or comprise one or more hormonal agents (e.g., androgen inhibitors), one or more alkylating agents, one or more anthracyclines, one or more cytoskeletal disruptors (e.g., microtubule targeting agents such as taxanes, maytansine and analogs thereof, of), one or more epothilones, one or more histone deacetylase inhibitors HDACs), one or more topoisomerase inhibitors (e.g., inhibitors of topoisomerase I and/or topoisomerase II), one or more kinase inhibitors, one or more nucleotide analogs or nucleotide precursor analogs, one or more peptide antibiotics, one or more platinum-based agents, one or more retinoids, one or more vinca alkaloids, and/or one or more analogs of one or more of the following (e.g., that share a relevant anti-proliferative activity). In some particular embodiments, a chemotherapeutic agent may be or comprise one or more of Actinomycin, all-trans retinoic acid, an Auiristatin, Azacitidine, Azathioprine, Bleomycin, Bortezomib, Carboplatin, Capecitabine, Cisplatin, Chlorambucil, Cyclophosphamide, curcumin, Cytarabine, Daunorubicin, Docetaxel, Doxifluridine, Doxorubicin, Epirubicin, Epothilone, Etoposide, Fluorouracil, Gemcitabine, Hydroxyurea, Idarubicin, Imatinib, Irinotecan, Maytansine and/or analogs thereof (e.g., DM1) Mechlorethamine, Mercaptopurine, Methotrexate, Mitoxantrone, a Maytansinoid, Oxaliplatin, Paclitaxel, Pemetrexed, Teniposide, Tioguanine, Topotecan, Valrubicin, Vinblastine, Vincristine, Vindesine, Vinorelbine, and combinations thereof. In some embodiments, a chemotherapeutic agent may be utilized in the context of an antibody-drug conjugate. In some embodiments, a chemotherapeutic agent is one found in an antibody-drug conjugate selected from the group consisting of: hLL1-doxorubicin hRS7-SN-38, hMN-14-SN-38, hLL2-SN-38, hA20-SN-38, hPAM4-SN-38, hLL1-SN-38, hRS7-Pro-2-P-Dox, hMN-14-Pro-2-P-Dox, hLL2-Pro-2-P-Dox, hA20-Pro-2-P-Dox, hPAM4-Pro-2-PDox, hLL1-Pro-2-P-Dox, P4/D1 0-doxorubicin, gemtuzumab ozogamicin, brentuximab vedotin, trastuzumab emtansine, inotuzumab ozogamicin, glembatumomab vedotin, SAR3419, SAR566658, BIIB015, BT062, SGN-75, SGN-CD19A, AMG-172, AMG-595, BAY-94-9343, ASG-5ME, ASG-22ME, ASG-16M8F, MDX-1203, MLN-0264, anti-PSMA ADC, RG-7450, RG-7458, RG-7593, RG-7596, RG-7598, RG-7599, RG-7600, RG-7636, ABT-414, IMGN-853, IMGN-529, vorsetuzumab mafodotin, and lorvotuzumab mertansine. In some embodiments, a chemotherapeutic agent may be or comprise one or more of famesyl-thiosalicylic acid (FTS), 4-(4-Chloro-2-methylphenoxy)-N-hydroxybutanamide (CMH), estradiol (E2), tetramethoxystilbene (TMS), S-tocatrienol, salinomycin, or curcumin. In certain embodiments, chemotherapeutic agents and/or oncolytic therapeutic agents for anti-cancer treatment comprise (e.g., are) biological agents such as tumor-infiltrating lymphocytes, CAR T-cells, antibodies, antigens, therapeutic vaccines (e.g., made from a patient's own tumor cells or other substances such as antigens that are produced by certain tumors), immune-modulating agents (e.g., cytokines, e.g., immunomodulatory drugs or biological response modifiers), checkpoint inhibitors) or other immunologic/pharmacologic agents (e.g., PI3Kγ-selective inhibitor targeting myeloid cells or IPI-549). In certain embodiments, immunologic agents include immunoglobins, immunostimulants (e.g., bacterial vaccines, colony stimulating factors, interferons, interleukins, therapeutic vaccines, vaccine combinations, viral vaccines) and/or immunosuppressive agents (e.g., calcineurin inhibitors, interleukin inhibitors, TNF alpha inhibitors). In certain embodiments, hormonal agents include agents for anti-androgen therapy (e.g., Ketoconazole, ABiraterone, TAK-700, TOK-OOl, Bicalutamide, Nilutamide, Flutamide, Enzalutamide, ARN-509).

Marker: A “marker”, as used herein, refers to an entity or moiety whose presence or level is a characteristic of a particular state or event. In some embodiments, presence or level of a particular marker may be characteristic of presence or stage of a disease, disorder, or condition. To give but one example, in some embodiments, the term refers to a gene expression product that is characteristic of a particular immune cell type, immune cell subclass, activation of immune cells, and/or polarization of immune cells. Alternatively or additionally, in some embodiments, a presence or level of a particular marker correlates with activity (or activity level) of a particular signaling pathway, for example that may be characteristic of a particular class of immune cells. The statistical significance of the presence or absence of a marker may vary depending upon the particular marker. In some embodiments, detection of a marker is highly specific in that it reflects a high probability that the cell is of a particular immune cell type and/or subclass. In certain embodiments, a marker is a cytokine. In certain embodiments, a marker is a chemokine. In certain embodiments, a marker is a receptor. In certain embodiments, a marker is a genetic marker (e.g., mRNA, RNA) indicative of activation of a gene.

Pharmaceutical composition: As used herein, the term “pharmaceutical composition” refers to an active agent, formulated together with one or more pharmaceutically acceptable carriers. In certain embodiments, active agent is present in unit dose amount appropriate for administration in a therapeutic regimen that shows a statistically significant probability of achieving a predetermined therapeutic effect when administered to a relevant population. In certain embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

Radiolabel: As used herein, “radiolabel” refers to a moiety comprising a radioactive isotope (radioisotope) of at least one element. Exemplary suitable radiolabels include but are not limited to those described herein. In certain embodiments, a radiolabel is one used in positron emission tomography (PET). In certain embodiments, a radiolabel is one used in single-photon emission computed tomography (SPECT). In certain embodiments, radioisotopes comprise ^{99m}Tc, ¹¹¹In, ⁶⁴Cu, ⁶⁷Ga, ¹⁸⁶Re, ¹⁸⁸Re, ¹⁵³Sm, ¹⁷⁷Lu, ⁶⁷Cu, ¹²³I, ¹²⁴I, ¹²⁵I, ¹¹C, ⁴³N, ¹⁵O, ¹⁸F, ¹⁸⁸Re, ¹⁵³Sm, ¹⁶¹Ho, ¹⁴⁹Pm, ⁹⁰Y, ²¹³Bi, ¹⁰³Pd, ¹⁵⁹Gd, ¹⁴La, ¹⁹⁸Au, ¹⁹⁹Au, ¹⁶⁹Yb, ¹⁷⁵Yb, ¹⁶⁵Dy, ¹⁶⁶Dy, ¹⁰⁵R, ¹¹¹Ag, ⁸⁹Zr, ²²⁵Ac, ¹⁹²Ir, and ⁸⁹Zr.

Small molecule inhibitor: As used herein, term “small molecule inhibitor” includes small molecule inhibitors used for the treatment of cancer. For example, with respect to prostate cancer, a small molecule inhibitor can be any small molecular inhibitor described in the art, e.g., see https://www.drugs.com/condition/prostate-cancer.html, the contents of which is incorporated by reference herein in its entirety. In embodiments, a small molecule inhibitor refers to a class of inhibitors that can target myeloid-derived suppressor cells (MDSCs)/Tregs, including colony stimulating factor 1 (CSF-1)-receptor inhibitors and indoleamine 2,3-dioxygenase (IDO) inhibitors, that can be used to block tumor-infiltrating MDSCs in B16IDO tumors, in addition to enhancing anti-tumor T cell responses. In some embodiments, a small molecule inhibitor is a PI3Kγ-selective inhibitor targeting myeloid cells. In some embodiments, a small molecule inhibitor is IPI-549.

Subject: As used herein, the term “subject” includes humans and mammals (e.g., mice, rats, pigs, cats, dogs, and horses). In many embodiments, subjects are mammals, particularly primates, especially humans. In certain embodiments, subjects are livestock such as cattle, sheep, goats, cows, swine, and the like; poultry such as chickens, ducks, geese, turkeys, and the like; and domesticated animals particularly pets such as dogs and cats. In certain embodiments (e.g., particularly in research contexts) subject mammals will be, for example, rodents (e.g., mice, rats, hamsters), rabbits, primates, or swine such as inbred pigs and the like.

Therapeutically effective amount: as used herein, is meant an amount that produces the desired effect for which it is administered. In certain embodiments, the term refers to an amount that is sufficient, when administered to a population suffering from or susceptible to a disease, disorder, and/or condition in accordance with a therapeutic dosing regimen, to treat the disease, disorder, and/or condition. In certain embodiments, a therapeutically effective amount is one that reduces the incidence and/or severity of, and/or delays onset of, one or more symptoms of the disease, disorder, and/or condition. Those of ordinary skill in the art will appreciate that the term “therapeutically effective amount” does not in fact require successful treatment be achieved in a particular individual. Rather, a therapeutically effective amount may be that amount that provides a particular desired pharmacological response in a significant number of subjects when administered to patients in need of such treatment. In certain embodiments, reference to a therapeutically effective amount may be a reference to an amount as measured in one or more specific tissues (e.g., a tissue affected by the disease, disorder or condition) or fluids (e.g., blood, saliva, serum, sweat, tears, urine, etc.). Those of ordinary skill in the art will appreciate that, in certain embodiments, a therapeutically effective amount of a particular agent or therapy may be formulated and/or administered in a single dose. In certain embodiments, a therapeutically effective agent may be formulated and/or administered in a plurality of doses, for example, as part of a dosing regimen.

Therapeutic agent: As used herein, the phrase “therapeutic agent” in general refers to any agent that has a therapeutic effect and/or elicits a desired biological and/or pharmacological effect when administered to a subject.

Treatment: As used herein, the term “treatment” (also “treat” or “treating”) refers to administration of a therapy that partially or completely alleviates, ameliorates, relives, inhibits, delays onset of, reduces severity of, and/or reduces incidence of one or more symptoms, features, and/or causes of a particular disease, disorder, and/or condition. In some embodiments, such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition.

Tumor: As used herein, the term “tumor” refers to an abnormal growth of cells or tissue. In some embodiments, a tumor may comprise cells that are precancerous (e.g., benign), malignant, pre-metastatic, metastatic, and/or non-metastatic. In some embodiments as discussed herein, a tumor is associated with, or is a manifestation of, a cancer. In some embodiments as discussed herein, a tumor may be a solid tumor.

Drawings are presented herein for illustration purposes, not for limitation.

### BRIEF DESCRIPTION OF THE FIGURES

The foregoing and other objects, aspects, features, and advantages of the present disclosure can become more apparent and better understood by referring to the following description taken in conjunction with the accompanying drawings, in which:

FIGS. 1A-1B depict low dose (100 nM) C′ dot treatment of macrophages leads to M1 polarization in the absence of ferroptosis. FIG. 1A shows BMDM treated with 10 or 100 nM C′ dots for 24 hours show upregulation of M1 polarization markers (iNOS and TNFα) and downregulation of M2 markers (CD206, Arg1 and IL-10) by immunofluorescence (left) and qRT-PCR (middle graph and right heat map). FIG. 1B shows treatment of BMDM with 100 nM C′ dots does not induce cell death. Images show control (left) and treated (right) BMDM from time-lapse analysis after 24 h. Right graph shows quantification of cell survival for control (black) and treated (gray) BMDM after 24 h. Data show percent surviving cells out of 15 microscopic fields of view from multiple independent experiments.

FIG. 2 depicts a western blot showing that low dose (10 and 100 nM) C′ dot treatment of macrophages leads to upregulation of ferritin heavy chain (FTH1), consistent with iron loading activity. This activity is blocked with the iron-chelating agent, deferoxamine (DFO).

FIG. 3 depicts a plot showing concentration-dependent gene expression modulation by C′ dots in ovarian, prostate, and melanoma cancer cells. FIG. 3 also shows a heatmap of gene expression profiles of ID8, TRAMP-C2, B16-F10 cancer cells incubated with and without C′ dots (15 μM) for 3 days. Each quadrant represents an independent biological replicate.

FIGS. 4A-4B depict plots (FIG. 4A) and a schematic (FIG. 4B) of molecular and phenotypic characterization of VEGF-A low and VEGF-A high-expressing ID8 syngeneic ovarian peritoneal carcinomatosis models. FIG. 4A depicts survival curves for ID8 VEGF-A low (blue) and VEGF-A high (black) ID8 syngeneic ovarian peritoneal carcinomatosis models. FIG. 4B depicts a schematic of the sample collection strategy for the molecular analysis in FIGS. 4C-4D.

FIGS. 4C-4D depict plots showing gene expression profiles of tumor (FIG. 4C) and splenic (FIG. 4D) tissues harvested from VEGF-A low and VEGF-A high tumor-bearing mice relative to week 2 (W2) following inoculation of cells.

FIGS. 5A-5C depict plots and imaging showing dose-dependent cellular uptake and viability of C′ dot-exposed ID8 ovarian cancer cells. FIG. 5A shows a plot depicting viability of particle-exposed ID8 cells as a function of C dot concentration over 7 days. FIG. 5B shows images of concentration-dependent uptake of PEG-Cy5-C′ dots in ID8 ovarian cancer cells over a 24-hour incubation period. Concentration-dependent internalization of PEG-Cy5-C′ dots using confocal microscopy. Hoechst dye (blue), C′ dot (red). FIG. 5C shows a plot depicting mean fluorescence intensity (MFI) as a function of particle concentration using flow cytometry. (Scale bar=10 μm).

FIG. 6 depicts a plot showing concentration and time-dependent gene expression modulation by C′ dots in ID8 VEGF-A low ovarian cancer cells. FIG. 6 also shows a heatmap of gene expression profiles of ID8 VEGF-A low cancer cells incubated with C′ dots at increasing concentrations (0, 0.1, 1, 15 μM) for 1, 3 and 6 days. Each quadrant represents an independent biological replicate.

FIG. 7 depicts plots showing cytotoxicity of CAR T cells as a function of C′ dot concentration. Viability of luc+ ID8 cells after incubation with C′ dots at increasing concentrations (0-15 μM) for 24 hours, followed by co-culture with media alone for 24 hours (black bar), CD19 non-specific CAR T cells (blue bar), or 4H11 (anti-hMUC16)-CAR T cells, using a 3:1 (red bar) or 5:1 effector-to-target cell ratio (green bar). Data represents analyses of biological replicates/group (n=4), mean±s.e.m. (non-parametric 2-way ANOVA).

FIGS. 8A-8B depict a bar graph (FIG. 8A) and images (FIG. 8B) showing treatment with αMSH-PEG-Cy5-C′ dots synergizes with amino acid starvation to kill ID8 cells through ferroptosis. FIG. 8A shows a bar graph that depicts percent cell death of ID8 cells cultured for 24 or 48 hours in full or amino acid (AA)-free medium, in the presence or absence of 15 μM αMSH-PEG-Cy5-C′ dots. Data show percentages out of 500 cells from five independent microscopic fields of view; error bars show s.d. FIG. 8B shows images that depict representative fields of view from time-lapse imaging of control and αMSH C′ dot-treated cells, cultured in AA-free medium.

FIGS. 9A-9B show that C′ dot treatment modulates transcription profiles in ID8 orthotopic tumors. FIG. 9A shows a schematic of the dosing strategy for non-targeting PEG-Cy5-C′ dots or saline vehicle. FIG. 9B shows plots depicting gene expression profiles for tumor, ascites and spleen in mice treated with C′ dots relative to those treated with saline vehicle, measured as fold changes (n=4).

FIGS. 10A-10F depict plots showing preferential localization of PEG-Cy5-C′ dots to ID8 cells in ascites specimens. FIGS. 10A-10F show plots depicting flow cytometry analysis of C′ dots in ID8 and CD45+ cells in ascites and splenic specimens. Data show representative histograms (FIGS. 10A, 10C, 10E) and MFI measurements (FIGS. 10B, 10D, 10F) of n=5 biological replicates per group, mean±s.e.m. (t-test).

FIGS. 11A-11K show plots depicting that C′ dots promote maturation of DCs, enhance activation of T cells and decrease M2 macrophage polarization. FIGS. 11A-11E show plots depicting analysis of CD40 in DCs in ascites and splenic specimens by MPFC. FIGS. 11F-11I show plots depicting analysis of IFNγ in CD8+ T cells in ascites and splenic specimens by MPFC. FIGS. 11J-11K show plots depicting analysis of Arg1 and CD206 in macrophages in ascites and splenic specimens by MPFC. Data represent analyses of n>3 biological replicates for treated and control groups, mean±s.e.m. (t-test).

FIG. 12A shows a schematic of a dosing strategy for combination of C′ dots with CAR T cells in ID8 VEGF-low and VEGF-high animal models, according to an embodiment of the present disclosure.

FIG. 12B shows data depicting percent overall survival as a function of days post tumor implantation for mice administered saline (black curve), C′ dots (grey curve second from the left), CAR T cells (blue curve second from the right), and dual treatment (both C′ dots and CAR T cell) (red, first curve from right).

FIGS. 13A-13F show characterization of a lead alpha-MSH peptide-linker conjugate. Structural schematics of representative alpha-MSH peptides. FIG. 13A shows prototype rhenium (Re)-cyclized peptide (curve with open circles in FIG. 13E). FIG. 13B, FIG. 13C each show second generation peptides with reduced hydrophobicity containing PEG2 and one Ahx spacer: (FIG. 13B) PEG2-Ahx-d-Lys-alpha-MSH (yellow closed circle in FIG. 13E); and (FIG. 13C) PEG2-d-Lys-Ahx-alpha-MSH (green triangle in FIG. 13E). FIG. 13D shows lactam-cyclized peptide (purple upside down triangle in FIG. 13E). FIG. 13E and FIG. 13F show IC₅₀ determination of MC1-R targeting Re-cyclized and lactam-cyclized peptides.

FIG. 14 shows images depicting induction of ferroptosis in B16-F10 cells. (Left image) C′ dot-treated B16F10 cells in amino acid (AA)-deprived media undergo ferroptosis with wave-like propagation. Left and middle images show DIC and SYTOX Green fluorescence; left image shows live cells, right image shows SYTOX-positive dead cells after ferroptosis. (Right image) Nuclei of ferroptotic cells in left image, pseudocolored to indicate relative timing of cell death by time-lapse microscopy.

FIGS. 15A-15C show C′ dots inhibit melanoma tumor growth via ferroptotic induction and pro-inflammatory responses. FIG. 15A shows a graph depicting B16-F10 growth inhibition in mice following n=3 doses of i.v.-injected 1st generation alpha-MSH-PEG-C′ dots (36 nmoles (60 mM), red closed circles), as against alpha-MSH-PEG-C′ dot+liproxstatin-1 (gray triangle), and saline vehicle (blue closed circle), initiated when tumors reached ˜50 mm3 (day 0), repeated on days 3 and 6 thereafter. FIG. 15B shows imaging depicting H&E staining of tissues after final dose. FIG. 15C shows histograms that graphically depict alterations of macrophage (Iba1+) and pan/helper/cytotoxic (CD3/CD4/CD8+) T cell populations in the TME by IHC after particle+/−liproxstatin-1 or vehicle exposure. Data represents analyses of n=4 mice per group, mean±s.e.m. *p<0.05 (non-parametric 2-way ANOVA).

FIG. 16A shows tumor growth inhibition in cohorts of B16-F10 mice injected with 3 doses of anti-PD-1+ first generation alpha-MSH-C′ dots (36 nmoles (60 mM)) (green), anti-PD-1 alone (gray), alpha-MSH-C′ dots alone (red), or saline vehicle (blue) after tumor volumes reached 50 mm³. Data represent mean±s.e.m. **** saline-PD1: P=0.0001; **** saline-alpha-MSH: P=0.0001; **** saline-(anti-PD1+alpha-MSH): P<0.0001; **PD1-(anti-PD1+alpha-MSH): P=0.0017; **alpha-MSH-(anti-PD1+alpha-MSH): P=0.0017.

FIG. 16B shows alteration of pan/helper/cytotoxic (CD3/CD4/CD8+) T cell populations in the TME by IHC after treatment with αMSH-C′ dots±anti-PD1, anti-PD1, or vehicle (‘C’).

FIG. 16C shows plots of pan/helper/cytotoxic (CD3/CD4/CD8+) T cell populations in the TME based on IHC (FIG. 16B). Data reflects analyses of n=4 mice/group, mean+s.e.m. *P<0.05, **P<0.01, ****P<0.001 (non-parametric one-way ANOVA).

FIG. 17 shows a bar graph depicting upregulation of gene expression signatures in non-targeted particle-exposed B16-GM cells. Gene expression signatures driving STING-, TLR-(DAMPS)-, and IFN-related pathways in B16-GMCSF cells as a function of particle concentration (72-hr incubation), displayed as fold changes (log 2 transform) over media alone (control).

FIGS. 18A-18D show bar graphs depicting C′ dot uptake drives proinflammatory and immune-related responses. FIG. 18A shows concentration-dependent uptake of C′ dots in B16-GMCSF cells over a 72-h period accompanied by (FIGS. 18B-18C) upregulation of Type I (FIG. 18B) and Type 2 IFN (FIG. 18C), (FIGS. 18D, 20A) increased IFNAR1 expression relative to controls, (FIG. 20B) increased ROS, (FIG. 20C) reductions in PD-L1, and (FIG. 20D) increased ATP secretion over 48-h. Studies were performed in triplicate and analyzed using FC (*P<0.05; **P<0.01; ***P<0.005; ****P<0.0001).

FIGS. 19A-19D depict images showing enhanced immunogenicity of tumor and immune cell populations derived from C′ dot-exposed B16-GMCSF models. FIG. 19A show cytoplasmic dsDNA (bright puncta) in untreated (top panel) and C′ dot-exposed (15 mM) tumor cells (bottom panel) over 72 h using IF with nuclear counterstaining (DAPI). Scale bar=8 μm. FIG. 19B-19C show cytosolic or cytoplasmic dsDNA (FIG. 19B) and nuclear and cytoplasmic genomic DNA (FIG. 19C) quantified using Quant-IT assay and images in (FIG. 19A). FIG. 19D shows that no contributions were found as a result of the inflammasome, noting marked reductions in caspase-1 from controls (72 h).

FIGS. 20A-20D show bar graphs depicting C′ dot uptake drives proinflammatory and immune-related responses. FIG. 20A shows increased IFNAR1 expression relative to controls, FIG. 20B shows increased ROS relative to controls, FIG. 20C shows reductions in PD-L1 relative to controls, and FIG. 20D shows increased ATP secretion over 48-h relative to controls. Studies were performed in triplicate and analyzed using FC (*P<0.05; **P<0.01; ***P<0.005; ****P<0.0001).

FIGS. 21A-21C depict data showing enhanced immunogenicity of tumor and immune cell populations derived from C′ dot-exposed B16-GMCSF models. FIG. 21A shows extracellular caspase-1 in untreated and C′ dot-treated cells (15 mM) via luminescence. FIG. 21A shows particle-exposure (48 h) and phenotyping of cultured BMDMs from B16-GMCSF mice using FC. FIG. 21B shows quantification of secreted IFNg from splenocytes cultured with CD3/CD28+/−C′ dots, and FIG. 21C shows percentage of live B16-GMCSF cells co-cultured with pmel-1 T cells (+/−) CD3/CD28, with and without C′ dots, by flow cytometry. Studies were performed in triplicate. (*P<0.05; **P<0.01; ****P<0.0001).

FIGS. 22A-22C and FIGS. 23A-23B show tumor infiltrating lymphocyte and myeloid cell population changes due to C′ dot treatment increases activation and decreases inhibitory T cell and myeloid cell populations. B16-GMCSF tumor mice were triply injected with C′ dots (36 nmoles; 60 mM) every 3 days when tumor volumes reached 50 mm³. Tumors were harvested 96 h after the final dose. Single cell dissociates were stained for surface markers, and samples run in triplicate. All populations reflect % CD45⁺ cells. Both anti-tumor and suppressive myeloid cells (FIG. 22B, FIG. 22C), CD8⁺ T cells (FIG. 22A, FIG. 23A), and CD4⁺ T cells (FIG. 23B) were examined; *=p<0.05, ***=p<0.005.

FIGS. 24A-24G show data depicting impact of antibody-based cell depletion of CD8+/CD4+/NK and myeloid cells on tumor growth, PRR pathway activation, and anti-tumor cytokine production. FIGS. 24A-24B show tumor growth inhibition in cohorts of B16-GMCSF mice injected with 3 doses of PEG-C′ dots (red, 36 nmoles (60 mM) or saline (blue) after tumor volumes reached 50 mm³ using C57/B6 (FIG. 24A) and NSG (FIG. 24B). IHC and plots of STING (FIG. 24C) and PD-L1 (FIG. 24D) expression in harvested tumor tissue specimens from (FIG. 24A). FIG. 24E, FIG. 24F show antibody-based depletion of T cell and NK cells (FIG. 24E) and myeloid cells (FIG. 24F) in B16-GMSCF tumors (C57/B6 mice) using the same particle-based treatment regimen as in (FIG. 24A, FIG. 24B). FIG. 24G shows gene and cytokine production in ex vivo tumor and serum (inset) samples from (FIG. 32A) **P<0.01, ***P<0.05, ****P<0.001 (non-parametric one-way ANOVA). Scale bar=250 m.

FIG. 25 show PSMA-targeting peptide-linkers, according to embodiments of the present disclosure. For example, FIG. 25 shows peptide-linker sequences synthesized with varying degrees of hydrophilicity.

FIGS. 26A-26B show competitive cell binding. FIG. 26A shows competitive binding curves for PSMAi-peptide and representative PSMAi-peptide-linker conjugates: DFO-PSMAi-PEG-Cy5.5-C′dots, DOTA-PSMAi-PEG-Cy5.5-C′ dots, and NOTA-PSMAi-PEG-Cy5.5-C′ dots using PSMAi-(67Ga)NOTA-peptide in LNCaP cells. Data mean+s.d. of 3 replicates. FIG. 26B shows IC50 (×10⁻⁹ μM) values for all constructs.

FIG. 27A shows chemical structures of (i) prototype PSMAi, (ii) PSMAi-PEG2, and (iii) PSMAi-2Nal-Tran. FIG. 27B shows cell binding of prototype PSMAi-C′ dots and new PSMAi-PEG2-C′ dots.

FIGS. 28A-28E show data and images depicting in vitro PSMA-targeted C′ dot uptake and specificity in PCa cells. Cellular binding/uptake of PSMAi-PEG-Cy5-C′ dots in PCa cells for a range of particle concentrations by flow cytometry (FIG. 28A) and confocal microscopy (100 nM, 4 h incubation time). Each data point represents mean+s.d. of 3 replicates. FIGS. 28B-28E show specific binding of same PSMAi-targeted C′ dots. Mean fluorescence intensity (MFI) in LNCaP (FIG. 28C), MyC-CaP (FIG. 28D) and PC-3 cells (FIG. 28E) exposed to PSMA-targeted C′ dots without and with addition of anti-PSMA antibody. **=p<0.01, ***=p<0.001.

FIGS. 29A-29B show data depicting gene expression and cytokine/chemokine expression profiles of PSMAi-PEG-Cy5-C′ dot-treated MyC-CaP cells. FIGS. 29A-29B show (FIG. 29A) gene and (FIG. 29B) cytokine/chemokine expression profiles of supernatant from MyC-CaP cells exposed to up to 15 mM PSMAi-C′ dots for 24 hours.

FIG. 30 shows a plot that depicts that C′ dots upregulate genes in C2 cells derived from the TRAMP model involved in cell stress response, antigen presentation, Type I interferons and antioxidant genes in a dose-dependent manner.

FIG. 31 shows data and images depicting in vivo targeted (bottom graph) biodistribution and (left inset) PET imaging of ⁸⁹Zr-DFO-PSMAi-PEG-Cy5-C′ dots in LNCaP (left; n=5) and PC-3 (right, n=5) tumor-bearing male mice with corresponding tumor uptake, tumor-to-muscle (T/M) ratios, and (right inset) histology and autoradiography.

FIGS. 32A-32B show a schematic, images, and data depicting that C′ dots modulate gene expression profiles in the Hi-Myc model. FIG. 32A shows a schematic of the Hi-Myc-driven murine PCa model and dosing strategy for C′ dots (top row) with corresponding H&E, cMyc, Ki-67, and PSMA staining at 24 weeks (bottom row). FIG. 32B shows a gene expression profile for tumor tissues in animals treated with 3 doses of i.v.-injected PSMAi-C′ dots, PSMAi-C′ dots+ICB, ICB, and vehicle. Significant fold changes are indicated by values above 2 (horizontal bar).

FIG. 33 shows data depicting that C′ dots upregulate ISGs and modulate tumor/immune cell markers in the Hi-Myc model with and without ICB. FIG. 33 shows a gene expression profile of ISGs for animals treated with 3 doses of i.v.-injected PSMAi-C′ dots, PSMAi-C′ dots+ICB, ICB, and vehicle. Significant fold changes are indicated by values above 2 (horizontal bar)

FIGS. 34 and 35 show data depicting that tumor infiltrating lymphocyte (FIG. 34) and myeloid cell population (FIG. 35) changes due to PSMA-targeted C′ dot treatment in the Hi-Myc transgenic prostate cancer model with and without ICB. Hi-Myc mice were i.v.-injected with C′ dots followed by i.p.-administered dual ICB. Tumors were harvested 96 hours after the final dose. Dissociated single cells were then stained for indicated markers and analyzed by flow cytometry. Samples were run in triplicate. *=p<0.05, **=p<0.01, ***=p<0.005, ****=p<0.001.

FIG. 36 shows data depicting that C′ dots upregulate ISGs and modulate tumor/immune cell markers in the Hi-Myc model with and without ICB. FIG. 36 shows that cMyc, Ki-67, and PDL1 were quantified via IHC. Samples were run in triplicate. *=p<0.05, **=p<0.01.

FIG. 37 shows a possible mechanism for C′ dot RT-driven immune activation (Schema 1) to be activation of cell stress/cell death programs, which can lead to cGAS/STING activation, among other pathways, resulting in NF-kB/IRF/Type I IFN responses.

### DETAILED DESCRIPTION

It is contemplated that methods, compositions, and processes of the claimed invention encompass variations and adaptations developed using information from the embodiments described herein. Adaptation and/or modification of the methods, compositions, and processes described herein may be performed, as contemplated by this description.

Throughout the description, where methods, compositions, and processes are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited steps.

It should be understood that the order of steps or order for performing certain action is immaterial so long as the invention remains operable. Moreover, two or more steps or actions may be conducted simultaneously.

The mention herein of any publication, for example, in the Background section, is not an admission that the publication serves as prior art with respect to any of the claims presented herein. The Background section is presented for purposes of clarity and is not meant as a description of prior art with respect to any claim.

Documents are incorporated herein by reference as noted. Where there is any discrepancy in the meaning of a particular term, the meaning provided in the Definition section above is controlling. In certain embodiments, the silica-based nanoparticle platform comprises ultrasmall nanoparticles or “C′ dots,” which are fluorescent, organo-silica core shell particles that have diameters controllable down to the sub-10 nm range with a range of modular functionalities. C′ dots are described by U.S. Pat. No. 8,298,677 B2 “Fluorescent silica-based nanoparticles”, U.S. Publication No. 2013/0039848 A1 “Fluorescent silica-based nanoparticles”, and U.S. Publication No. US 2014/0248210 A1 “Multimodal silica-based nanoparticles”, the contents of which are incorporated herein by reference in their entireties. Incorporated into the silica matrix of the core are near-infrared dye molecules, such as Cy5.5, which provides its distinct optical properties. Surrounding the core is a layer or shell of silica. The silica surface is covalently modified with silyl-polyethylene glycol (PEG) groups to enhance stability in aqueous and biologically relevant conditions. These particles have been evaluated in vivo and exhibit excellent clearance properties owing largely to their size and inert surface. Among the additional functionalities incorporated into C′ dots are chemical sensing, non-optical (PET) image contrast and in vitro/in vivo targeting capabilities, which enable their use in visualizing lymph nodes for surgical applications, and melanoma detection in cancer. Moreover, the disclosure of International Patent Application No. PCT/US19/66944, filed Dec. 17, 2019, entitled, “Inducing Favorable Effects on Tumor Microenvironment via Administration of Nanoparticle Compositions,” is hereby incorporated by reference in its entirety

Headers are provided for the convenience of the reader—the presence and/or placement of a header is not intended to limit the scope of the subject matter described herein.

Recent discoveries focus on two improved self-therapeutic capabilities of the particles themselves without the need for attached cytotoxic drugs: (i) nanoparticle (e.g., C′ dot)-induced cell death programs, such as ferroptosis, an iron-driven mechanism of propagated cancer cell death and the active engagement; (ii) priming of immune cells directly within the tumor microenvironment (TME), which internalize these particles; (iii) induction of proinflammatory gene signatures that highlight nanoparticle use as an exogenous agonist of IFN-related pathways; (iv) downregulation of genes, genes, inhibitory immune cell populations (eg, Tregs), T cell receptors (eg, TIM3, LAG3), and checkpoint proteins (eg, PD1, PD-L1) driving immune suppressive activities; (v) direct activation of innate and immune effector cell populations, and (vi) polarization of macrophages to M1 phenotypes. For example, regarding C′ dot-induced ferroptosis, it was confirmed that the features of this propagated ‘multicellular’ mode of cell death to offer distinct benefits over other modes of cell death (e.g., apoptosis), e.g., those that cause single-cell death and are non-propagative in nature. In the former case, the spatial and temporal pattern of wave-like cell death spreading induced by C′ dots, the only clinically-translated agent, was non-random in nature, while this was not the case for other agents exhibiting a non-propagative ‘single-cell’ death mode (e.g., GPX4, an enzyme which increases anti-ferroptotic activity).

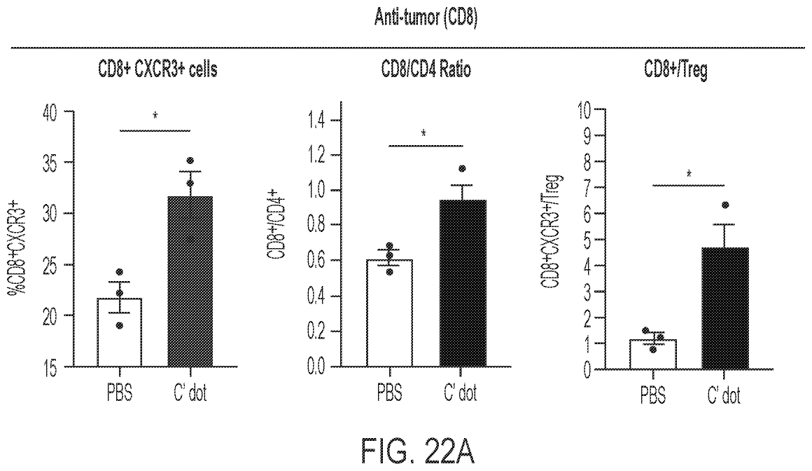

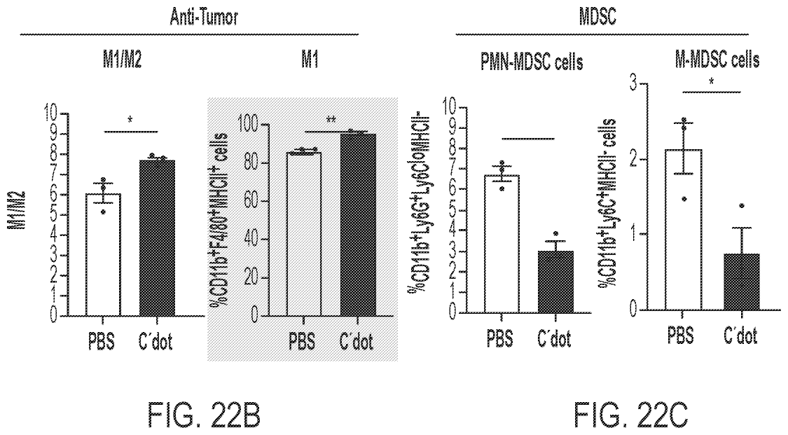

Ferroptosis plays a significant role in driving anti-tumor responses in various cell types at sites of metabolic deprivation. It has been shown that the compositions described herein do not inhibit the enzymatic activity of GPX4 (FIG. 22), a key antioxidant and ferroptosis-inhibiting enzyme, suggesting that iron delivery into cells is the primary mechanism by which the described compositions induce ferroptosis. Immune-related cell death has also resulted the use of the described compositions, for example, in B16-GMCSF cells. In other contexts, key regulators such as inflammatory caspases and/or regulators of alternative death mechanisms, such as pyroptosis or necroptosis, may be contributory, examined by inhibitor-based or genetic studies.

Ferroptosis may drive, under some conditions, inflammatory responses within the TME of specific tumor types, which may be attributed to intracellular iron delivery from the described compositions that promotes lipid peroxidation and rupture of the cell membrane. Intracellular organelles such as lysosomes may rupture during ferroptosis execution as a result of lipid peroxidation and/or osmotic swelling, and may contribute to the execution of cell death or to inflammatory signaling.

These discoveries have led to new methods that harness the potent anti-cancer activities of these particle-based tools to address limitations of cancer immunotherapies, while enhancing their therapeutic effectiveness in solid tumor malignancies. Moreover, as CD8+ T cells are known to also regulate ferroptosis during immunotherapy, such effects are expected to synergize with particle-induced biological responses.

The methods employ particle-mediated enhancement of anti-tumor immunity and cell death in immunosuppressed tumor microenvironments (TME). The methods improve treatment outcomes under conditions of tumor-induced immunosuppression and immune resistance by harnessing the favorable biological properties of a set of self-therapeutic ultrasmall (e.g., diameter less than 8 nm) silica-organic core-shell nanoparticles with tunable size, composition, surface chemical properties, and functionalities. Used as treatment tools in combination with cancer immunotherapy, these particles augment the capabilities of immunotherapeutic agents, overcoming properties of the tumor microenvironment (TME) limiting efficacious outcomes.

In certain embodiments, the administered nanoparticles exhibit and promote intrinsic pro-inflammatory and cancer cell death-inducing properties.

The silica nanoparticles described herein are found to modulate multiple class-specific gene expression signatures across a variety of cancer cell types. For example, class-specific up-regulation of many gene expression signatures was observed for particle-exposed cell lines, as against media controls. Single-cell RNA sequencing (scRNA-seq) can be used to further identify immunomodulation capabilities of silica nanoparticles on cell population dynamics in the TME, to discriminate among heterogeneous cell populations within the TME, and/or to identify rare cell populations that respond to the silica nanoparticles and play a role in therapeutic outcomes.

Described herein are methods of particle-mediated enhancement of anti-tumor immunity and cell death in immunosuppressed tumor microenvironments (TME) to improve treatment outcomes. Nanoparticles are designed to (i) exhibit and promote intrinsic pro-inflammatory and cancer cell death-inducing properties. The nanoparticles synergize with immunotherapies to enhance efficacy and augment responsiveness of immune checkpoint blockade (ICB) antibodies or to expand the window of treatment for administering engineered cellular therapies (e.g., chimeric antigen receptor (CAR) T-cell therapy) post-inoculation of tumor cells. The combinatorial strategies may, for example, enhance immunogenicity, reverse immune suppression, and limit immune resistance—all factors that improve the effectiveness of these immune-based therapies. Also described herein are predictive and prognostic biomarkers (e.g., expression markers, immune phenotypes, histologic markers) for use in these applications.

These innovative approaches enhance anti-tumor immunity and cell death in solid tumor malignancies by exploiting the intrinsic therapeutic properties of a suite of precision-tuned ultrasmall core-shell silica particle-based tools. Such particles can serve as combinatorial partners for existing standard and experimental agents used for cancer immunotherapy. Without the need for anti-tumor or immunomodulatory drugs, these particles have led to polarization of macrophages towards an M1 phenotype, induced proinflammatory gene signatures; downregulated genes, inhibitory immune cell populations (e.g., Tregs), T cell receptors (e.g., TIM3, LAG3), and checkpoint proteins (e.g., PD1, PD-L1) driving immune suppressive activities; activated innate and immune effector cell populations and other immune-related components, induced ferroptosis, as well as promoted radiation-induced cytotoxicity following the surface-attachment of therapeutic nuclides. Based on these properties and a body of preliminary data, these self-therapeutic particles are expected to play a critical role in promoting proinflammatory responses, effector cell kill, and one or more cell death mechanisms (e.g., ferroptosis, immune-related cell death) in combination with standard of care CAR T-cells, ICB, and myeloid cell-targeting pharmacologic inhibitors. The present disclosure describes that these new, clinically translatable combinatorial strategies can yield one or more additive or synergistic and sustainable treatment responses beyond that of the particles alone in cases of tumor-induced immunosuppression: (i) augment ferroptosis- and/or immune-related cytotoxicity; (ii) activate the TME; and (iii) reverse immune suppression, which thereby (iv) limits/improves immune resistance. Given that these particles are renally cleared, multi-dose regimens can be employed as part of a dose escalation scheme to maximize specific therapeutic responses without off-target effects (e.g., reticuloendothelial system, RES)-advantages of the platform. Even at very low doses (55 picomoles), particles are observed to be therapeutically active, noting modest, but statistically significant, survival benefits relative to vehicle controls. Therefore, the potential utility of these self-therapeutic particle probes offers a more attractive and effective approach of optimizing and translating cancer nanotherapies over traditional paradigms, particularly for cases in which resistance develops, first-line treatments fail, and/or alternative strategies are not available. In addition, the drawbacks that can arise with the use of cancer immunotherapies may potentially be overcome by the addition of these particles in order to enhance immunogenicity of an otherwise “cold” TME to improve treatment response, decrease expression of immune resistance markers, inhibit recruitment of immunosuppressive factors and/or cell populations, as well as reduce dose-limiting toxicity.

One innovation of certain embodiments is the use of self-therapeutic targeted silica nanoparticles (e.g., C′ dots) in combination with external beam radiation (EB-RT) or alpha-/beta-emitting molecular radiotherapy (e.g., peptide-based) and immunotherapy. Self-therapeutic C′ dots used with EB-RT, for example, may demonstrate both radiation-induced cell kill, as well as activation of the TME due to modulatory effects of the base particle; the combination with immunotherapy will provide unprecedented opportunities to tailor the attack on tumor tissues. Such multi-pronged approaches with C′ dots can therefore lead to multiple therapeutic responses that occur concomitantly, each with a distinct mechanism of action. For combinatorial C′ dots, radiotherapy and immune ICB, this can include priming/activating of the TME, particle-induced ferroptosis, radiation-induced cytotoxicity, downregulation of inhibitory markers, and immune effector cell kill. Since C′ dots themselves can lead to more than one therapeutic response, which may all arise within a very short time frame, this may mitigate potential side effects for the patient.

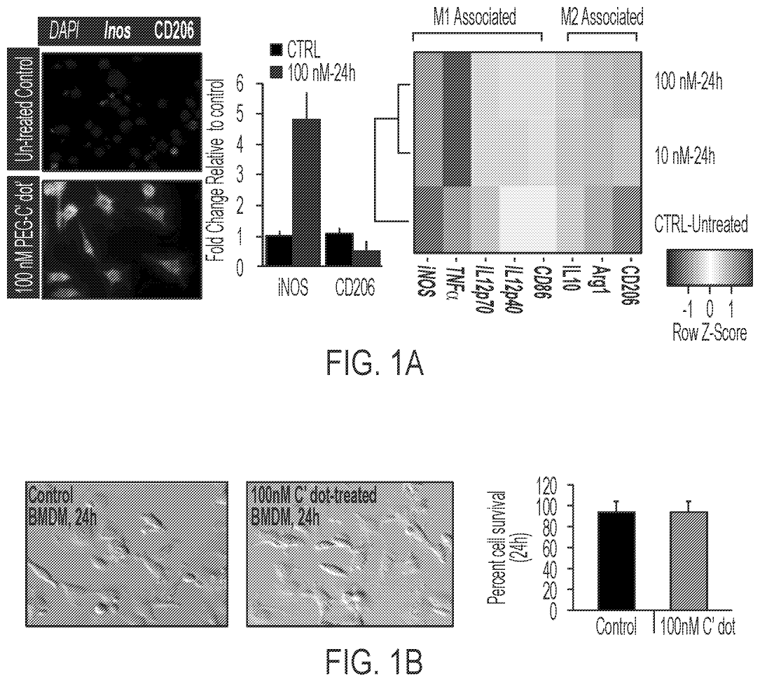

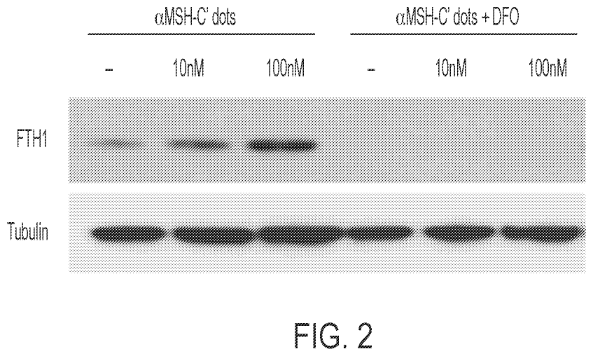

For immune cells, treatment with low-dose particle concentrations (10 or 100 nM) are sufficient to polarize macrophages (FIG. 1A), but may not induce ferroptosis (FIG. 1B). Under these conditions, ferritin heavy chain (FTH1) upregulation has also been observed (FIG. 2), which could be blocked by the iron-chelating agent, deferoxamine (DFO), consistent with an increased level of intracellular iron due to particle-mediated delivery. Based on this data, and without wishing to be bound to a particular theory, the present disclosure hypothesizes that iron delivery by C′ dots into macrophage lysosomes could be the initiating signal that induces M1 polarization.

### Examples: Particle-Driven Modulation of Cancer Gene Expression Profiles

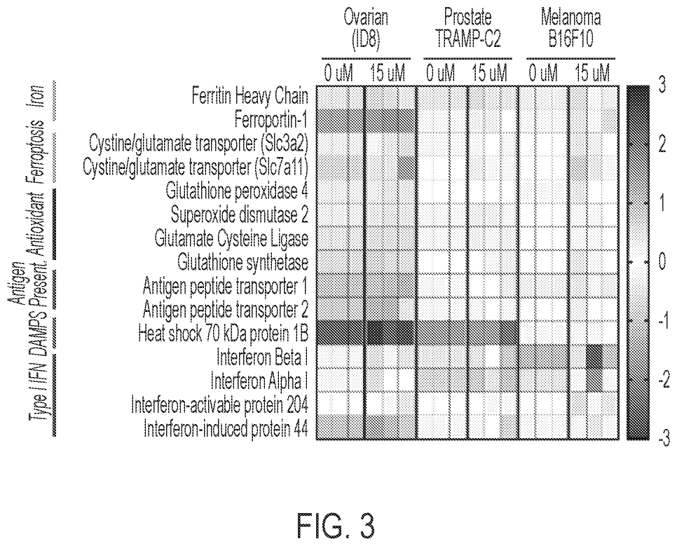

In addition to polarizing macrophages, C′ dots were also found to modulate multiple class-specific gene expression signatures across a variety of cancer cell types being investigated. A diverse set of class-specific expression markers representing major histocompatibility complex (MHC) class-I antigen-presenting genes, damage-associated molecular patterns (DAMPs), type I interferon (IFN) responses, iron-related genes, ferroptosis-related genes, and antioxidant-related genes was compiled for surveying a wide array of tumor-immune responses in the described biological specimens using quantitative reverse transcription PCR (RT-qPCR). In vitro experiments were performed with a variety of tumor cell types, e.g., ovarian ID8 cells, transgenic adenocarcinoma (TRAMP)-C2 prostate cancer cells, and murine melanoma B16-F10 cells, which were incubated using a single high dose of C′ dots (15 μM) or media alone for 72 hours, followed by RT-qPCR analysis. As illustrated in FIG. 3, class-specific up-regulation of multiple gene expression signatures was observed for one or more particle-exposed cell lines, as against media controls, encompassing iron-related genes, antioxidant genes, antigen-peptide transporter genes, damage-associated molecular patterns (DAMPS), and type I IFN response genes. DAMP signatures (e.g., heat shock 70 kDa protein 1B) were strongly upregulated in both ID8 ovarian cancer, B16-GM cells, and TRAMP-C2 cells. Such molecules typically arise from dying, injured, or stressed cells that may secrete/release certain types of molecules, e.g., ATP and high mobility group protein B1 (HMGB1) or expose molecules which can become enriched on the outer leaflet of the plasma membrane (such as calreticulin, CRT, and heat shock proteins). These molecules function as either adjuvant or danger signals for the innate immune system. DAMPs are actively emitted from dying apoptotic cells and play a beneficial role in anti-cancer treatment responses due to their interaction with the immune system. Similarly, increased IFNα and IFNβ expression, combined with up-regulation of antigen peptide transporter genes, results in enhanced tumor-associated antigen (TAA) presentation, higher NK cell activation, and inhibition of Treg cells in the TME. In addition, iron-related gene signatures were upregulated, including ferritin heavy chain. It is shown that ferritin heavy chain is induced in response to particle treatment as cells increase iron binding capacity, a response to C′ dot-mediated iron delivery (FIG. 2). Ferroportin-1 is similarly upregulated as a likely cell export response to iron loading. Antioxidant signature genes included the enzymes superoxide dismutase, as well as GPX4, a known and specific inhibitor of ferroptosis that detoxifies lipid peroxides. Cystine/glutamate transporter light and heavy chain genes (Slc7a11 and Slc3a2) were also upregulated in ID8 cells and potentially in melanoma, suggesting that cystine import, a precursor of glutathione synthesis, is upregulated in some cancer cells as a likely antioxidant and anti-ferroptotic response to C′ dot administration. As immune checkpoint inhibition-based therapy has been shown to reduce expression of SLC7A11, this finding may underscore synergy between C′ dots and checkpoint blockade that both have the potential to induce ferroptosis, but by acting through different mechanisms.

Three Examples (see Example 1, see Example 2 see Example 3) are described which involve various embodiments described herein. Example 1 investigates whether the effectiveness of CAR T cell therapy can be sustained and enhanced, and the treatment window expanded in high-grade ovarian cancer using a combination of C′ dots and CAR T-cells. Example 2 describes limiting immune resistance by reversing tumor-induced immune suppression in melanoma models utilizing MC1-R-targeting (or non-targeting) C′ dots in combination with immunotherapies and targeted inhibitors. Example 3 describes compositions, methods, and systems to improve treatment outcomes and anti-tumor immunity in advanced prostate cancer (PC) models using new prostate-specific membrane antigen (PSMA)-targeted C′ dot compositions with either EB-RT or molecular (e.g., peptide-based radiotherapies/radioligands) that potentially synergize with checkpoint inhibitors while reducing off-target toxicity.

These three Examples describe preclinical particle designs that promote multiple separable intrinsic therapeutic capabilities of the platform and rationally combine a number of therapeutic options. The unique combination of therapeutic capabilities rely, in part, on the favorable pharmacokinetic profile exhibited by the platform. The strategies described by these Examples seek to identify self-therapeutic nanoparticles (e.g., non-targeted and/or targeted C′ dots) that, when combined with immunotherapies (CAR T-cells, immune ICB, pharmacologic inhibitors), and/or used in combination with either external beam radiation or alpha-/beta-emitting peptide radioligands, can improve therapeutic responses and anti-tumor immunity, limit immune resistance in solid tumors, reverse tumor-induced immunosuppression decreasing effectiveness of immunotherapies, and reduce debilitating off-target effects observed with RT alone.

In Example 1, non-targeting, PEG-coated C′ dots are coupled with therapeutic CAR T-cells to augment intratumoral immune responsiveness in high-grade ovarian cancer models. This, in turn, may enhance and sustain therapeutic effectiveness of CAR T-cell therapy, augment cytotoxicity, overcome antigen heterogeneity, reduce exhaustion, and/or expand the therapeutic window post-inoculation of ovarian cancer cells. In Example 2, MC1-R-targeting C′ dots are also used as part of a triple therapeutic regimen, each with a distinct mechanism of action, in order to enhance therapeutic efficacy, as well as reduce, or even overcome, immune resistance by promoting a more favorable TME for treating melanoma. To achieve this objective, for example, the combination of ferroptosis-inducing C′ dots with ICB may synergize to boost efficacy, further activate anti-tumor proinflammatory changes within the TME, and reduce inhibitory T cell receptors and/or immune checkpoint proteins. A small molecule inhibitor can then be added that targets immune suppressive myeloid cell populations in the TME; this agent has been found to previously enhance immunogenicity, leading to more efficacious treatment responses. In some embodiments, C′ dots are co-administered with either EB-RT or alpha-/beta-emitting molecular radiotherapy (i.e., peptide radioligands) prior to being combined with ICB.

### Example 1: Silica Nanoparticles and Therapeutic CAR T-Cells to Augment Intratumoral Immune Responsiveness in High-Grade Ovarian Cancer Models

Ovarian cancer (OC) remains the leading cause of mortality in women with gynecological malignancies, with more than 70% of patients presenting with advanced disease. Although early management with cytoreductive surgery and cytotoxic therapy is effective, mortality rates remain high, due to disease relapse. Advancing better therapeutic approaches in these tumors requires improved understanding of factors limiting treatment response, which includes an immunosuppressive tumor microenvironment.

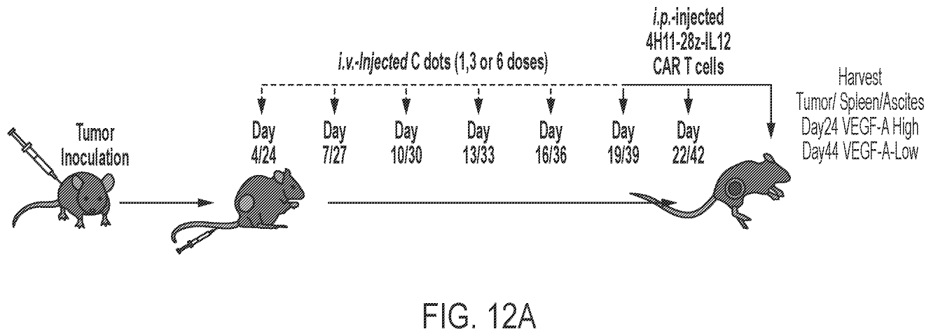

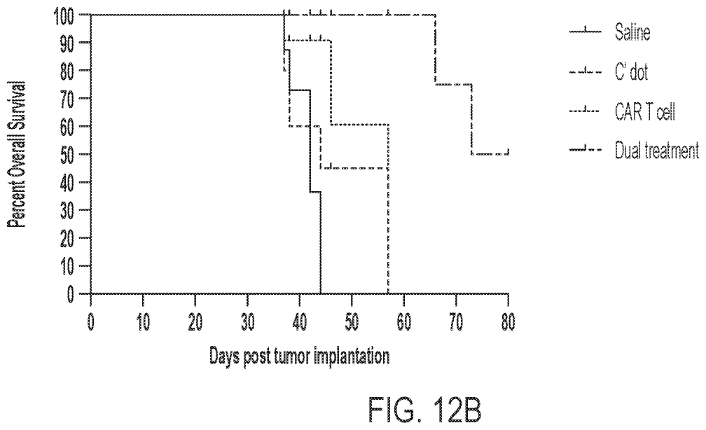

Particle localization to immune and cancer cell populations in the TME is related to changes in activation, cell death, and immunogenicity. The present disclosure describes that the ectodomain, MUC16^{ecto}, which is retained on the tumor cell surface, can be targeted with antibodies, targeted T cells, or targeted particle-based probes. Previously, a second-generation CAR, 4H11-28z, specific to the MUC16 ectodomain, and containing the cytoplasmic signaling domain of the CD28 co-stimulatory receptor was constructed. The 4H11 mAb, from which 4H11-28z is derived, binds to a majority of ovarian tumor clinical samples. Human 4H11-28z CAR T cells can successfully eradicate MUC16^{ecto}+ tumor cells in SCID-Beige mice. See FIGS. 12A-12B.

Experiments were conducted to determine how self-therapeutic C′ dots can be exploited to synergize with and overcome limitations of CAR T-cell therapy in MUC-16^{ecto} orthotopic OC models (e.g., ID8 model or the p53/K-ras GEM epithelial OC model, termed UPK10) to enhance cytotoxicity, efficacy, and expand the treatment window. See FIGS. 12 A-12B.

### Example 1A. Determine Tunable C′ Dot Structural Properties that Enhance Activation and Cytotoxicity of MUC16-Targeting, IL12-Secreting (MUC16ecto/IL-12) CAR T Cells and Immunogenicity of MUC16ecto OC Cells

In Example 1A, the present disclosure examines changes in structural properties and dosing of C′ dots which can alter (i) the biological properties of tumor cells derived from these MUC-16^{ecto} OC models, as well as immune cell populations, in addition to (ii) Example 1B, which describes favorably augmenting therapeutic efficacy and improving TME immune suppression in these orthotopic models by enhancing immune cell activation, cytotoxicity, immunogenicity, and pro-inflammatory phenotypic responses. In combination with CAR T cells, treatment responses in these models may be maximally enhanced, with expansion of the CAR T cell treatment window and abrogation of off-target toxicity.

Unlike other particle-based tools, the development of a single highly specialized ultrasmall particle-based agent that can be delivered systemically and can specifically engage ferroptosis, while also activating separable, ferroptosis-independent cytotoxic and/or pro-inflammatory mechanisms in the TME is previously undescribed. These therapeutic activities can, in turn, potentially reduce immunosuppressive factors that limit the normal functioning of effector cells and/or engineered immune cells, underscoring an additional advantage as described herein: leveraging these death-inducing mechanisms for treating relapsed and refractory solid tumors and their metastases, which also has relevance to treating human disease.

Experiments described by this Example screened for and identified certain structural properties of C′ dots that can (i) activate CAR T cells (or engineered cellular therapies) to enhance their cytotoxic responses; (ii) effectively polarize macrophages towards a pro-inflammatory phenotype, (iii) increase immunogenicity of MUC-16^{ecto} ID8 and UPK10 cells (as described herein), and (iv) maximize ferroptotic and/or other modes of cell death (e.g., immune-related), as a function of exposure time and C′ dot concentration. The present example also describes synthesis and characterization of non-targeted PEG-Cy5-C′ dots with varying structural properties in order to assess differential modulatory effects of these non-targeted particles on the biological properties of MUC-16^{ecto} ID8 cells and immune cell populations (e.g., macrophages, dendritic cells), and CAR T cells. The present example also describes evaluation of activation status and cytotoxicity of CAR T cells against C′ dot-exposed MUC-16^{ecto} ID8 cells.

### Example 1A-1. Control of Silica Core Composition

This example describes synthesis of a set of PEG-Cy5-C′ dots with varying silica core compositions. Aside from pure silica precursors, this example describes working with e.g., the monosodium salt of 3-(trihydroxysilyl)propyl-methylphosphonate (TPMP) to introduce phosphonate groups into the silica core (0-20 mole percent) with higher affinity to iron than pure silica (it is noted that the silica core is microporous with pore sizes less than 2 nm, where metal ions from e.g., iron, can be chelated, e.g., by surface Si—OH groups). Inductively coupled plasma (ICP) atomic emission spectroscopy can be used to quantify metal ion content.

### Example 1A-2. Control of Relative Silica Core and PEG Shell Size while Maintaining Overall Particle Size Below 6-7 nm

The water-based synthesis of C′ dots enables control of the silica core size at the level of a single atomic SiO2 layer. This example makes use of this exceptional degree of particle size control to generate a series of C′ dots with overall particle size maintained below the cut-off for renal clearance (e.g., below 6-7 nm) to reduce unwanted off-target uptake, while varying sizes of core and shell (varying PEG-silane length), respectively.

### Example 1A-3. Controlling Hydrophobic Particle Surface Patchiness

This example describes generation of a series of PEG-Cy5-C′ dots with varying hydrophobic “patchiness” utilizing Cy5 dyes with different net charge (e.g., negatively charged sulfo-Cy5(−)-maleimide dye (GE) or positively charged Cy5(+)-maleimide dye (Lumiprobe)), as well as variations of synthesis parameters (e.g., pH). Hydrophobic patchiness, which can arise from Cy5 dyes ending up on C′ dot surfaces (e.g., C′ dots encapsulate up to 4 Cy5 dyes), can be verified by the number of peaks seen by HPLC (e.g., 1-4) using 150 mm Waters Xbridge BEH C4 protein separation columns (300 Å pore size; 3.5 m particle size) and a water/acetonitrile mixture as mobile phase. This example describes generation of a series of PEG-Cy5-C′ dots with varying hydrophobic “patchiness”) utilizing Cy5 dyes with different net charge (e.g., negatively charged sulfo-Cy5(−)-maleimide dye (GE) or positively charged Cy5(+)-maleimide dye (Lumiprobe)), as well as variations of synthesis parameters (e.g., pH)(71, 72). Hydrophobic patchiness, which can arise from Cy5 dyes ending up on C′ dot surfaces (e.g., C′ dots encapsulate up to 4 Cy5 dyes), can be verified by the number of peaks seen by HPLC (e.g., 1-4) using 150 mm Waters Xbridge BEH C4 protein separation columns (300 Å pore size; 3.5 m particle size) and a water/acetonitrile mixture as mobile phase.

### Example 1B. Evaluate In Vivo Differences in Immune Modulation and Induction of Cell Death by C′ Dots to Determine Optimal Timing Intervals for Administration as Well as Maximize Anti-Tumor Responses Prior to Subsequent MUC16ecto/IL-12 CAR T Cell Injection

C′ dot candidates (Example 1A) can initially undergo screening pharmacokinetic (PK) studies to select candidates for studies in orthotopic models. Alterations in immune cell activation, pro-inflammatory phenotypes, and cell death can be monitored as a function of C′ dot dose, number of administered doses, and exposure time. Improved/optimized dosing strategies can then be applied to other OC models to maximize efficacy and reverse immune suppression. The present Example also describes in vivo serial monitoring of tumor burden and PK evaluations using C′ dots. The present Example also describes investigation of cellular and molecular profiles and tumor-induced immune suppression in ID8 tumors to improve treatment strategies and inform studies in alternative OC models (e.g., UPK10). Using these described strategies, the present example describes monitoring of dose-dependent changes in treatment response, immune modulatory status, and/or cell death using a C′ dot candidate in other OC orthotopic models.

### Example 1C. Assess Differential Anti-Tumor Immune Responses of CAR T Cells in Combination with C′ Dots to Improve Dosing Strategies Needed to Maximize Efficacy and Expand the Window for CAR T Cell Therapy

Optimal post-injection timing intervals needed to achieve maximum CAR T-cell mediated cytotoxicity in ID8 and UPK10 models can be established. Optimum C′ dot dosing strategies (Example 1B) can be implemented in both models to maximize efficacy, persistence, as well as aim to expand the CAR T cell therapeutic window. The present examples describe determination of an improved timing window for CAR T cell administration in the ID8 model. The present example also describes assessment of anti-tumor cytotoxic effects of CAR T-cells for particle-exposed and non-exposed tumors.

In Example 1, an anti-cancer strategy is investigated that combines nanomedicine with CAR T cell therapy to address the limitations of CAR T cell therapy. For example, it is found that C′ dots target and activate immune cells within the tumor microenvironment, as well as decrease suppressive myeloid cell populations. Enhanced cytotoxic responses of CAR T cells against ID8 cells was observed, which can synergize with the intrinsic therapeutic activities of C′ dots. In vitro studies using C′ dots showed synergistic activities in ID8 cells using T cells isolated from tumor-bearing mice, increasing their cytotoxicity. In addition, C′ dots activate immune cells in the TME toward an M1 pro-inflammatory phenotype and away from an M2 anti-inflammatory phenotype, independently of ferroptosis. Collectively, these new findings show that C′ dots exhibit intrinsic anti-cancer activities due to three properties: (1) an ability to specifically target cancer lesions; (2) an intrinsic ability to specifically induce the death of cancer cells; and (3) an intrinsic ability to activate the tumor-immune environment. The described results suggest that C′ dots can enhance cytotoxicity, immunogenicity, and activate the TME, as in Example 1, which demonstrates that C′ dots, in combination with CAR T cell administration, may be used to improve the efficacy and persistence of the latter, while expanding the CAR T cell therapeutic window.

The findings in the present application are based on the intrinsic therapeutic capabilities discovered for particle-based tools that (1) modulate the TME toward a pro-inflammatory phenotype, (2) increase T cell activation and cytotoxicity in the TME, and (3) target cancer cells directly for cell death through, for example, the mechanism of ferroptosis. The development of a single particle-based agent that can directly induce cancer cell death, in addition to activating the TME through separable mechanisms, promotes a more immunogenic TME prior to CAR T cell administration. These therapeutic activities, in turn, can potentially reduce immunosuppressive factors that limit the normal functioning of effector cells. Through the activation of type I interferon responses and other immunostimulatory pathways, C′ dots can potentially transform an immunosuppressive TME into one that enhances CAR T cell cytotoxicity, prolongs persistence, maximizes efficacy, and expands the window for treating these aggressive solid tumors. This paradigm shift marks a departure from traditional cancer nanomedicine drug delivery approaches which have relied on the conjugation of cytotoxic agents or, more recently, cytokines or adjuvants, to particle probes for enhancing efficacy or improving immunotherapeutic responses, respectively.

### Syngeneic Models of Murine Ovarian Peritoneal Carcinomatosis (e.g., ID8)

To understand how C′ dots can directly prime and activate the TME prior to administration of CAR T cells, syngeneic models of murine ovarian peritoneal carcinomatosis can be created using, for example, an ID8 epithelial ovarian cancer cell line or a p53/K-ras GEM epithelial OC cell line, UPK10. Given the heterogeneity of human primary high-grade ovarian tumors and their metastatic (e.g., peritoneal, omental) lesions, the present example seeks to examine differential pro-inflammatory and cytotoxic responses of these two metastatic models to C′ dots, with and without CAR T cells. ID8 ovarian cancer epithelial cells cause suppressive ascites fluid accumulation, a hallmark of late stage-advanced human ovarian carcinomatosis.

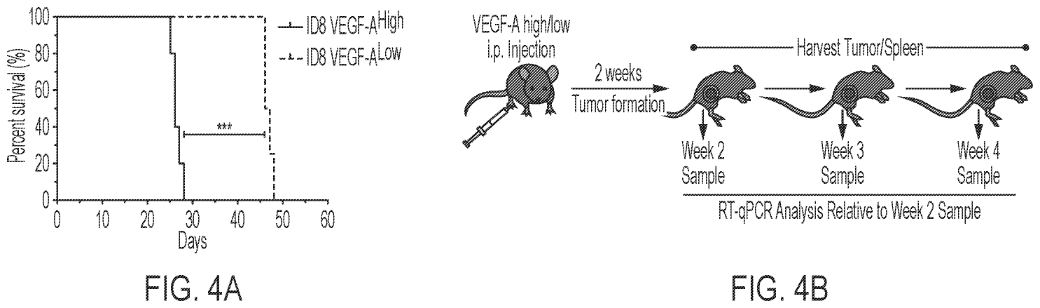

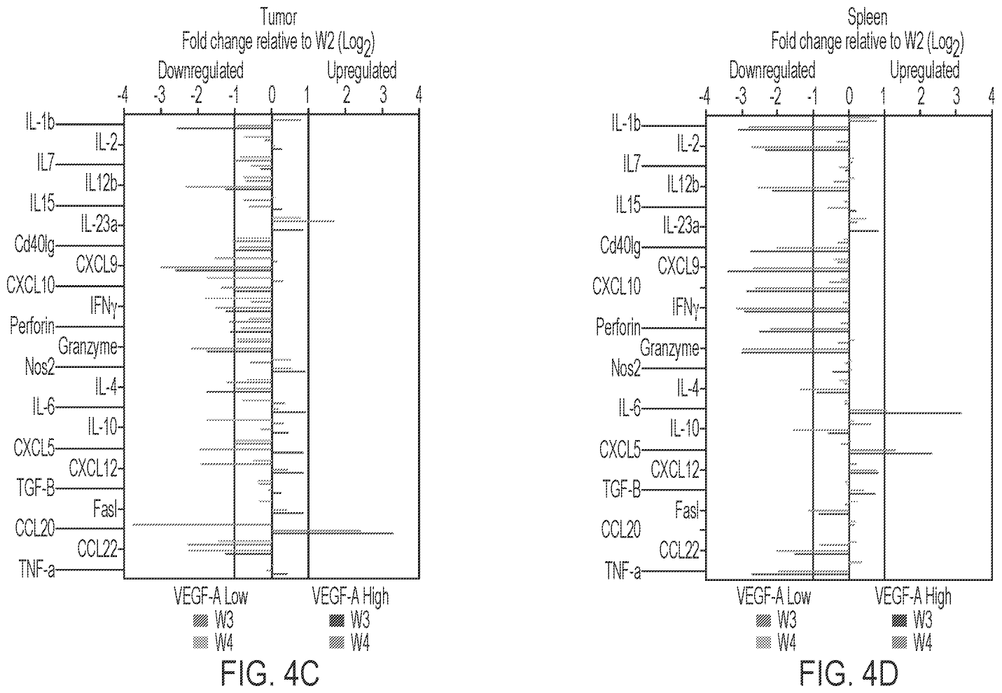

Orthotopic ID8 tumors were created in C57BL6 mice inoculated intraperitoneally (i.p.) for acquiring phenotypic and molecular signatures. Tumor growth was monitored by bioluminescence imaging (BLI) until detectable at about 2 weeks post-inoculation. Median survival time was about 47 days (data not shown) with formation of ascites at ˜4 weeks post-injection (p.i.). Transcription levels of three ID8 cell lines were measured—one parental (reference) and two VEGF-A+ transduced—as part of the described pilot studies, and showed distinct levels of VEGF-A expression: while the VEGF-A low cell line expressed mRNA levels comparable to that of the parental line, VEGF-A high cell line had 3.5 times higher levels. Molecular characterization of tumor and splenic tissues over a 3-week period was performed in both models using quantitative reverse transcription polymerase chain reaction (RT-qPCR) (FIG. 4B). Data was measured as a fold change in expression relative to week 2, the earliest time point at which tumor tissue could be harvested. In the case of the VEGF-A high model, gene expression analysis revealed that as the tumor grows, pro-inflammatory cytokines (e.g., IL1β, IL12b, IFNγ), chemokines associated with CD8+ T cell recruitment (e.g., CXCL9, CXCL10) and effector proteins (e.g., perforin, granzyme B) were significantly downregulated (FIG. 4C; blue bars), as indicated by fold changes extending beyond the vertical red bars (e.g., −1, 1). Immunosuppressive features were not restricted to the TME, as even larger changes were observed within splenic tissue (e.g., splenocytes) (FIG. 4D; blue bars). By contrast, gene expression changes in tumor tissue harvested from the VEGF-A low model were less pronounced (FIG. 4D; yellow bars), and not accompanied by significant expression changes in the spleen (FIG. 4C; yellow bars).

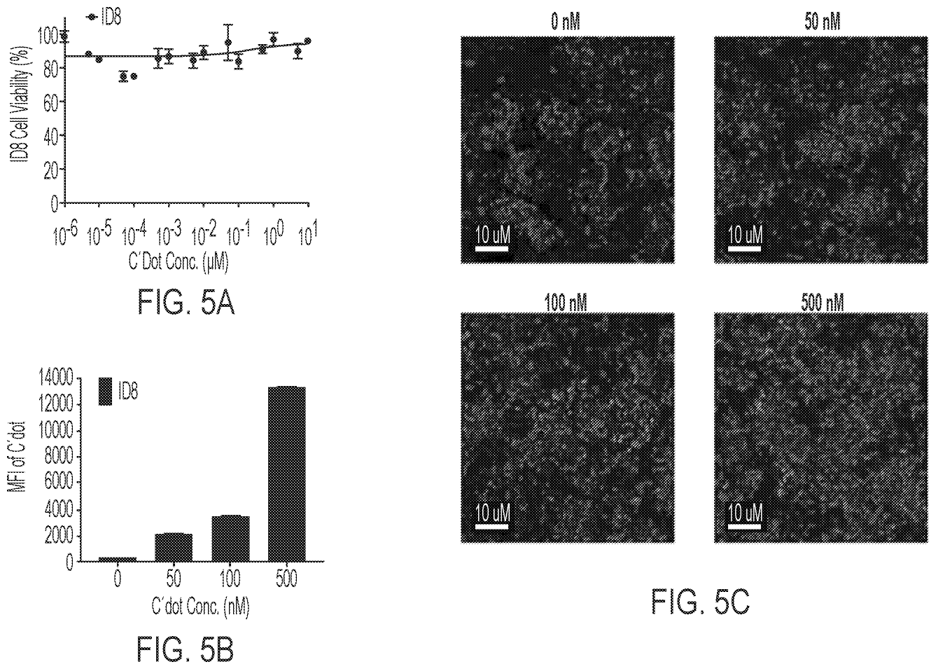

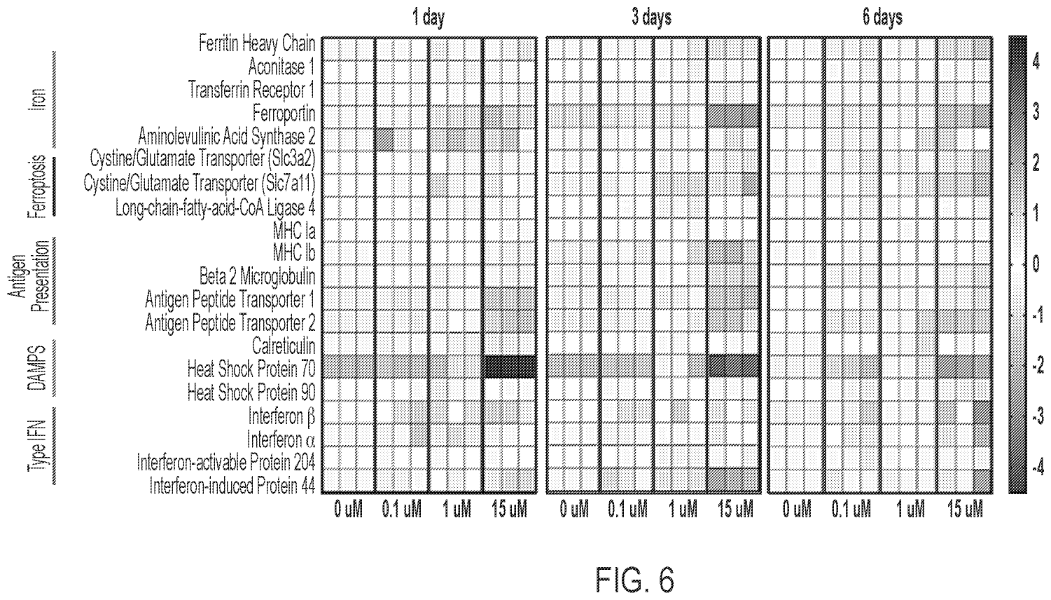

In FIGS. 5A-5C, cellular uptake of PEG-Cy5-C′ dots in ID8 cells, similar to that seen in prior C′ dot work with other cancer cell types, showed that particles were well tolerated across a wide range of concentrations (1 μM-10 μM) and incubation times (e.g., up to 6 days) without significant loss of viability (FIG. 5A). Further, C′ dot internalization by ID8 cells was observed over 24 hours using confocal microscopy (FIGS. 5B, 5C). Their uptake was found to rise in a concentration-dependent manner over a 24-hour period by flow cytometry, expressed as median fluorescence intensity (MFI) (FIG. 5C). To examine whether C′ dots increase the immunogenicity of, and induce ferroptosis in ID8 ovarian cancer cells, gene expression analyses of MUC-16^{ecto} ID8 cells exposed to increasing C′ dot concentrations (0.1, 1, 15 μM) for different incubation time periods (1, 3, 6 days) were performed (FIG. 6). Genes representing cellular responses in five major categories (iron metabolism, ferroptosis, major histocompatibility class (MHC) I antigen presentation, damage-associated molecular patterns (DAMPS) and type I interferon (IFN) responses were analyzed. The results were measured as a fold change in expression relative to the non-particle exposed group, and normalized using a log 2-transform. Upregulated gene expression in all five categories was observed as a function of C′ dot concentration (FIG. 6). Genes related to iron metabolism and homeostasis were upregulated (e.g., ferritin heavy chain and ferroportin). In addition, SLC3A2 and SLC7A11, two subunits of the cystine/glutamate antiporter system, which acts as a compensatory mechanism in response to increased oxidative stress, were substantially upregulated by C′ dots. Importantly, genes related to MHC class-I antigen presentation (e.g., MHC Ia, MHC Ib, TAP1, TAP2), DAMPS (e.g., Hsp70) and type I IFN responses (e.g., IFNα, IFNβ) were significantly upregulated in particle-exposed MUC-16^{ecto} VEGF-A low ID8 cells with increasing C′ dot concentrations, suggesting enhanced immunogenicity.

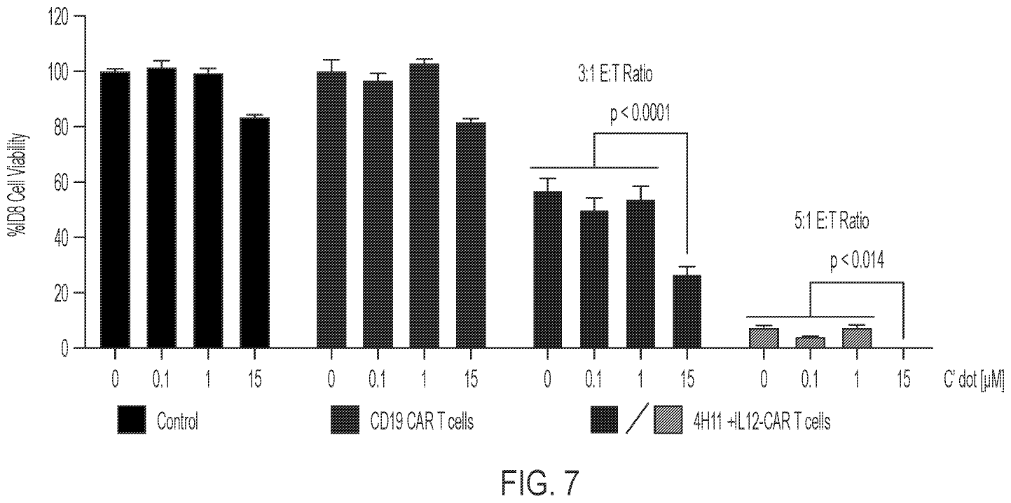

To test that C′ dots can synergize with MUC16^{ecto}/IL-12 CAR T cells (or referred to hereafter as simply CAR T cells) to increase anti-tumor cytotoxicity, a series of co-culture experiments were performed with CAR T cells and luciferase (luc+)-expressing ID8 cells, the latter with or without particle pre-treatment. Luc+ VEGF-A high/low ID8 cells were incubated with particles over a range of concentrations (e.g., 0, 0.1, 1, 15 μM) for 24 hours prior to exposure to CAR T cells (using different effector-to-target cell ratios of 5:1 and 3:1, see FIG. 7), and viability measured using a luciferase-based assay. Statistically significant cell viability losses were seen at the highest C′ dot concentrations after normalizing to results of a control group (e.g., no exposure to C′ dots or CAR T cells), which synergized with CAR T cells (FIG. 7; red, green bars). By contrast, no appreciable decrease in viability was observed when ID8 cells were pre-incubated with 15 μM C′ dots using a CD19-targeted CAR T cell control (FIG. 7; blue bars), as compared to the control group without CAR T cells (FIG. 7; black bars). This, and the described gene expression profiles, suggest that C′ dots can favorably modulate the immunogenicity of ID8 cells to increase effector cell kill, with the potential to activate the TME in ID8 orthotopic models prior to CAR T cell therapy.

Experiments can ascertain the extent to which limitations of present CAR T cell technologies and/or the immunosuppressive TME can be overcome by their combination with multi-therapeutic C′ dots. For example, experiments can assess differential modulatory effects of non-targeted C′ dots on the biological properties of MUC-16^{ecto} ID8 cells, macrophages, and CAR T cells. Selection of nanoparticle compositions can be based on their ability to effectively modulate multiple biological properties across various cancer and immune cell types.

### Gene Expression Profiling.

C′ dots were incubated with MUC16^{ecto} OC cells to select particles that induce upregulation of gene expression signatures related to iron metabolism (e.g., Fth1, Tf, Slc40a1), antioxidant enzymes (e.g., Nrf2, Nqo1, Gpx4, system Xc⁻ cystine/glutamate antiporter), MHC class I antigen presentation pathway (e.g., H2-D1, H2-T23, Tap1, Tap2), cell stress markers (e.g., Hsp70a1) and Type I interferons (e.g., Ifna1, Ifnb1). Changes in gene expression can be measured using RT-qPCR with TaqMan assays in duplexed reactions with controls (e.g., Gapdh). Results can be analyzed as a fold-change using the 2\hat{ }ΔΔCT method and a log 2-transform. C′ dots can also be incubated with immune cells (e.g., DCs, BMDMs) and 4H11-28z-IL12 CAR T cells to assess, e.g., maturation (e.g., Cd40) and T cell activation (e.g., Ifng), respectively.

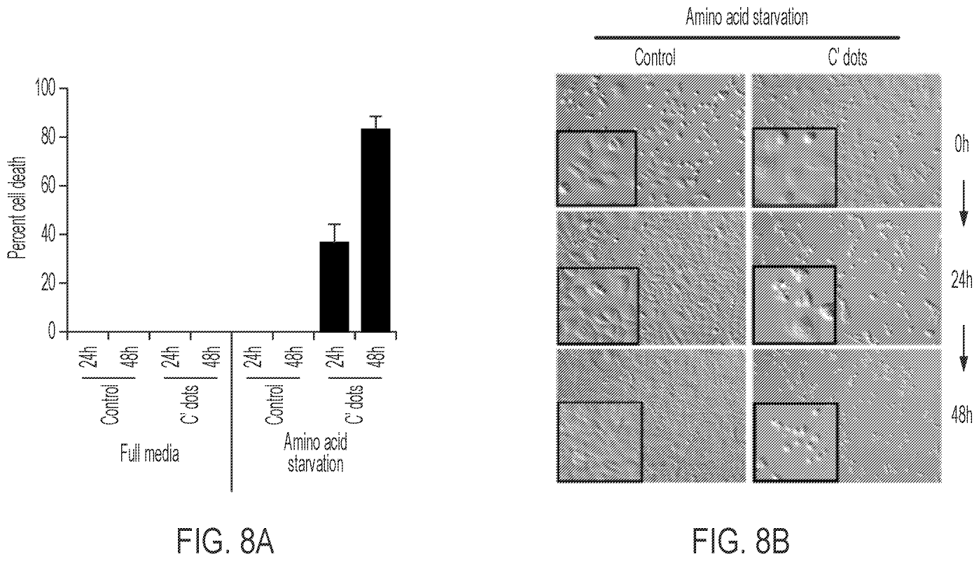

FIGS. 8A-8B demonstrates C′ dot-driven induction of ferroptosis has been observed in multiple human cancer cell lines, and is one of the iron-driven cell death programs by which C′ dots can inhibit tumor growth and/or promote regression. The present application seeks to identify nanoparticle compositions and the conditions under which they can maximally induce cell death. MUC16^{ecto} OC cells can be incubated over a range of increasing concentrations (100 nM-15 μM) and incubation times (24-72 hours), with and without an iron chelator (e.g., deferoxamine, DFO, 100 μM). Induction of cell death is evaluated by time-lapse microscopy and gene expression analysis to assess for viability, oxidative stress markers, and iron metabolism signatures as described herein. Results can be compared with those found for ID8 cells treated with other inducers (e.g., Erastin) or specific inhibitors (liproxstatin-1, 1 μM) of ferroptosis described in the art.

Experiments can evaluate activation status and cytotoxicity of CAR T cells against C′dot-exposed MUC-16^{ecto} OC cells. It is believed that the cytotoxic effects of CART cells can be augmented by pre-treating cancer cells with C′ dots.

CAR T cell proliferation and cytokine release profiles: Co-culture studies with MUC-16^{ecto} OC cells. To confirm that upregulation of gene expression profiles in MUC-16^{ecto} OC cells leads to enhanced CAR T cell activation, MUC-16^{ecto} OC cells are exposed to C′ dots over a range of increasing concentrations (100 nM-15 μM) and incubation times (24, 72 hours) prior to co-culture with CAR T cells. Proliferation of CAR T cells can be measured using cell proliferation tracking dyes and FC. Cytokine release profiles (e.g., IFNγ, TNFα, IL-2, -10, -12, TGFβ) can be assessed using Luminex assays.

CAR T Cell Cytotoxic Activity Against Luc+ MUC-16^{ecto} OC Cells with and without C′ Dots.

The data in FIGS. 8A-8B shows decreased MUC16^{ecto} ID8 cell viability when treated with combination of 15 μM C′ dots and CAR T cells, as compared to non-treated controls. In a parallel set of studies, C′ dot candidates were screened for their ability to synergize with CAR T cells to maximize cytotoxicity. Additional dose-response studies can be conducted using luc+ MUC-16^{ecto} ID8 cells (and other OC cells) by incubating cells with increasing particle concentrations (e.g., up to 15 μM) and a range of incubation times (over 72 hours) prior to CAR T cell exposure. Multiple effector-to-target cell ratios can also be examined in a similar manner to maximize cytotoxicity of CAR T cells. The viability of luc+ MUC16^{ecto} OC cells can be measured using a luciferase assay system. Results can be expressed as a percentage of cell viability relative to untreated controls.

### In Vitro Biological Endpoints for Proceeding to In Vivo Studies.

C′ dot probes can be selected for in vivo efficacy studies in Example 1B on the basis of findings across all in vitro cell assays and criteria for particle-exposed OC cells, including: an increase in Type I IFN (e.g., greater than or equal to 2-fold), an increase cell stress response markers and cell viability less than 10% (e.g., ferroptosis).

In Example 1B, cellular/molecular profiles and immune suppression in ID8 models are being investigated, prior to extending treatment strategies to a clinically relevant p53/K-ras UPK10 genetically engineered model that recapitulates human immune populations. The design of new precision treatment paradigms in high-grade ovarian cancer models rests on detailed knowledge of cellular and molecular profiles that alter the tumor-immune landscape. In vivo differences in immune modulation and induction of cell death in these OC models were assessed using nanoparticle compositions described herein and were evaluated to maximize anti-tumor responses prior to subsequent MUC16^{ecto}/IL-12 CART cell injection. For example, the ability of the described nanoparticle compositions, along with previously improved concentrations and incubation times, to modulate immune cell activation, proinflammatory phenotypes, and tumor-induced immune suppression in ID8 models were investigated in order to characterize studies in the clinically relevant p53/K-ras UPK10 OC model. The C′ dot dosing strategy in the ID8 model that demonstrates all of the following findings can serve as an initial dosing level for the p53/K-ras UPK10 model to maximize efficacy, with the aim of reversing immune suppression: (i) maximum fold changes (e.g., greater than 2) for specific genes (e.g., ferritin heavy chain, ferroportin, IFNα, IFNβ, IFNγ, among others), (ii) enhanced cytotoxic T cell activity, (iii) reduced immune suppression, and (iv) ferroptotic induction.

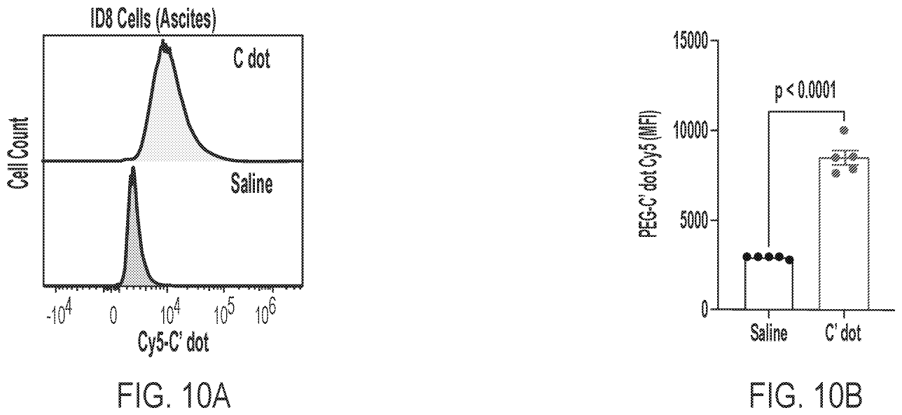

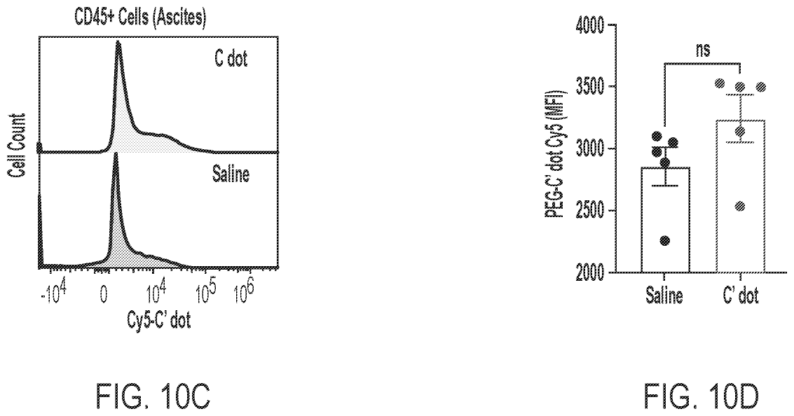

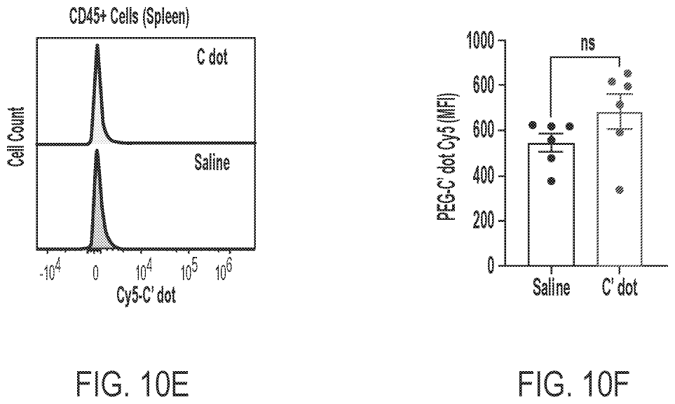

FC analysis was also run on harvested tumor and splenic specimens to evaluate particle localization at the cellular level in vivo. Tumor infiltrating leukocytes and ID8 cells were identified via CD45⁺ and GFP⁺ expression, respectively. Without wishing to be bound to any theory, preliminary analysis suggests that the majority of C′ dots concentrate on ID8 tumor cells as opposed to immune cells in the TME. Cy5 fluorescence of ID8 cells in tumors treated with C′ dots was significantly increased compared to saline-treated tumor controls (FIGS. 10A-10B). Tumor infiltrating leukocytes, by contrast, showed a slight fluorescence increase that was not significant (FIGS. 10C-10D). Additionally, Cy5 fluorescence of leukocytes isolated from splenic tissues of mice treated with saline or C′ dots was examined. Histograms and MFI quantification showed no difference between groups (FIGS. 10E-10F), further exemplifying the favorable PK profile of C′ dots in vivo.

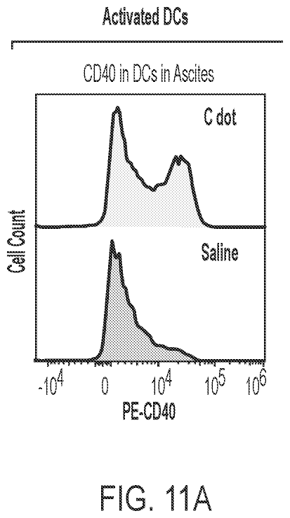

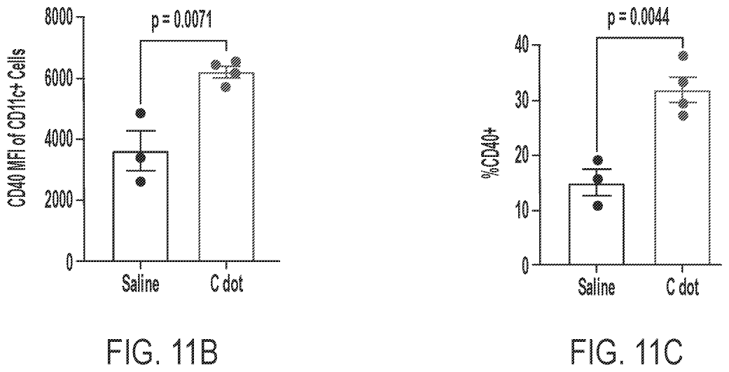

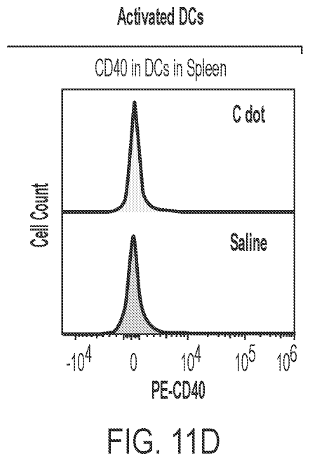

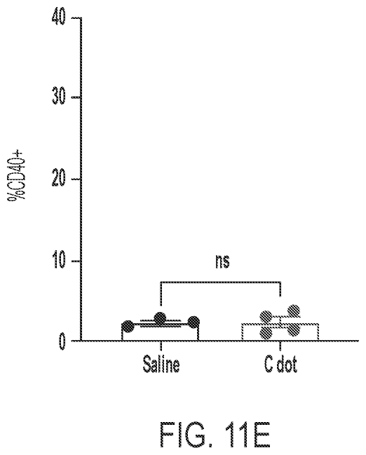

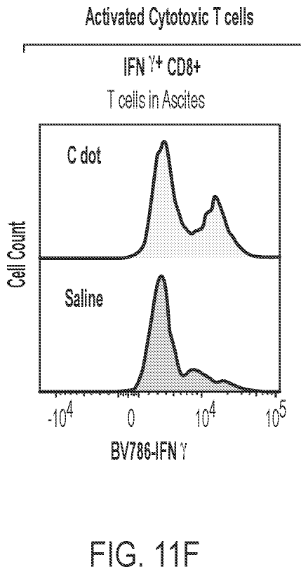

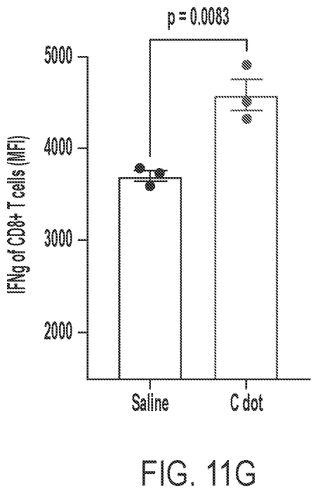

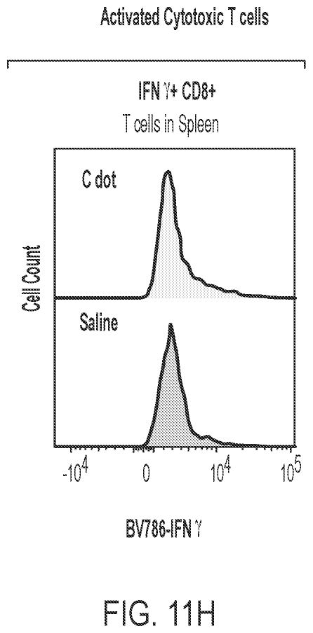

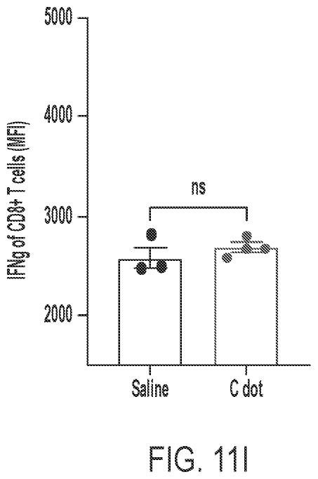

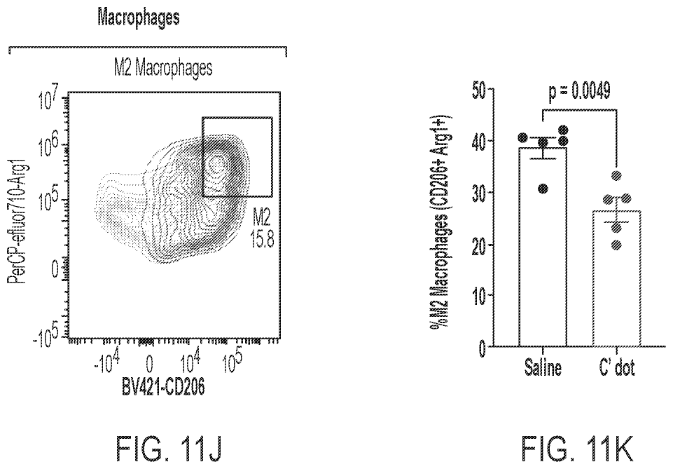

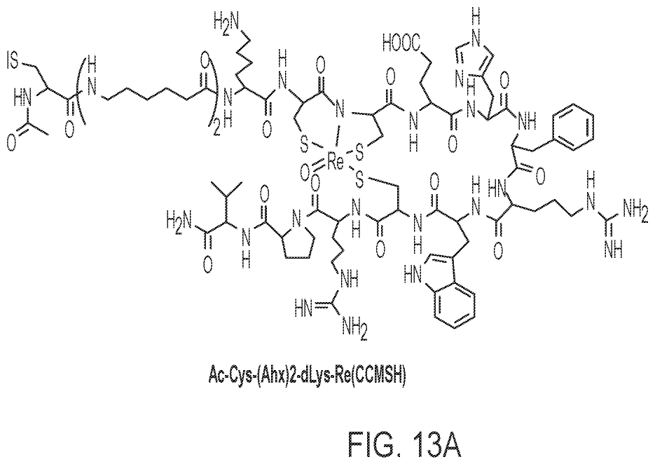

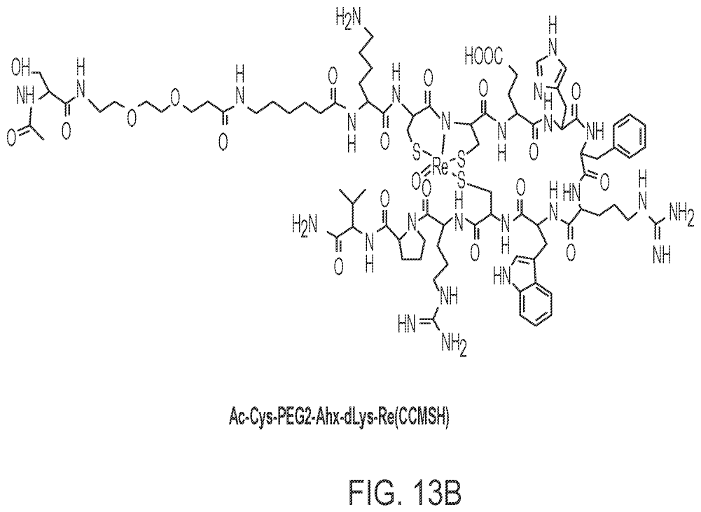

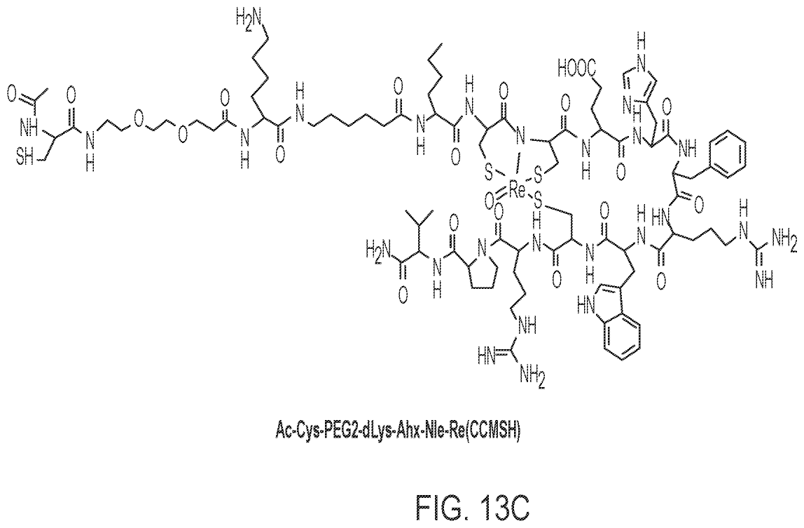

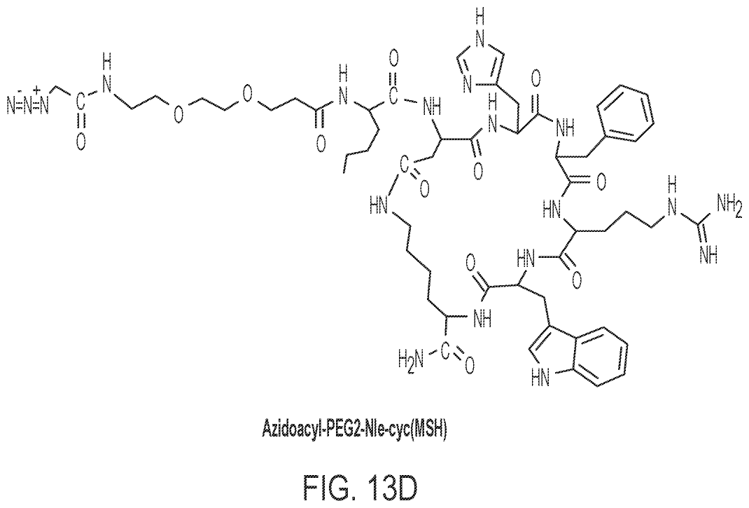

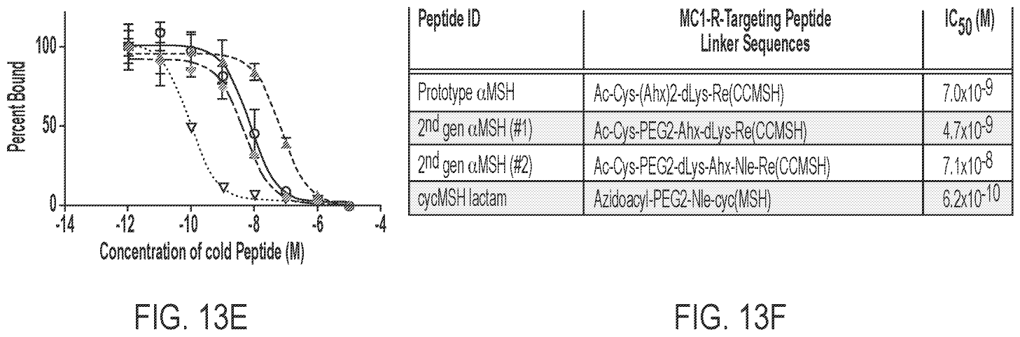

Moreover, immunophenotyping data was acquired using ascites and splenic tissue specimens harvested from the ID8 model, 96 hours after three, high-dose C′ dot injections, showed a statistically significant increase in DC maturation (FIGS. 11A-11C), accompanied by increased cytotoxic T cell activation (FIGS. 11F-11G) and a decrease in M2 anti-inflammatory macrophage polarization (FIGS. 11J-11K). No off-target effects were seen in the spleen (FIGS. 11D-11E; FIGS. 13D-13E). Importantly, without wishing to be bound to any theory, these changes—induced by untargeted particles—suggest that C′ dots have the potential to substantially activate anti-tumor immune responses in the TME without unwanted off-target effects, which may be further amplified once improved or if utilizing a targeted probe.

### Monitor Dose-Dependent Changes in Treatment Response, Immune Modulatory Status, and Cell Death in the UPK10 Model Using C′ Dots.

Orthotopic models exhibiting a spectrum of immune suppression highlights issues of intratumoral heterogeneity, which pose a major hurdle to effective treatment planning in the clinic. Obtained results can be used to inform selection of a single particle candidate to treat the immunosuppressive TME in the clinically-relevant p53/K-ras UPK10 model. Improved doses used to treat ID8 orthotopic tumors can be applied to the UPK10 model, enabling us to investigate differential immunomodulatory effects and cytotoxicity across models.

### Transcription Profiling and Cytokine Release Assays in Biological Specimens.

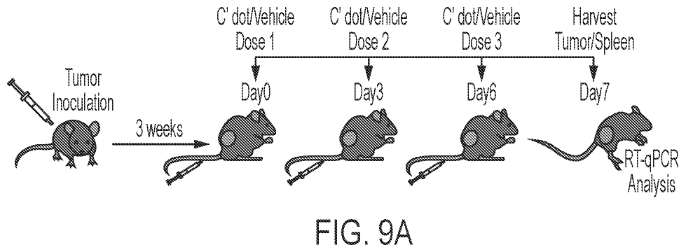

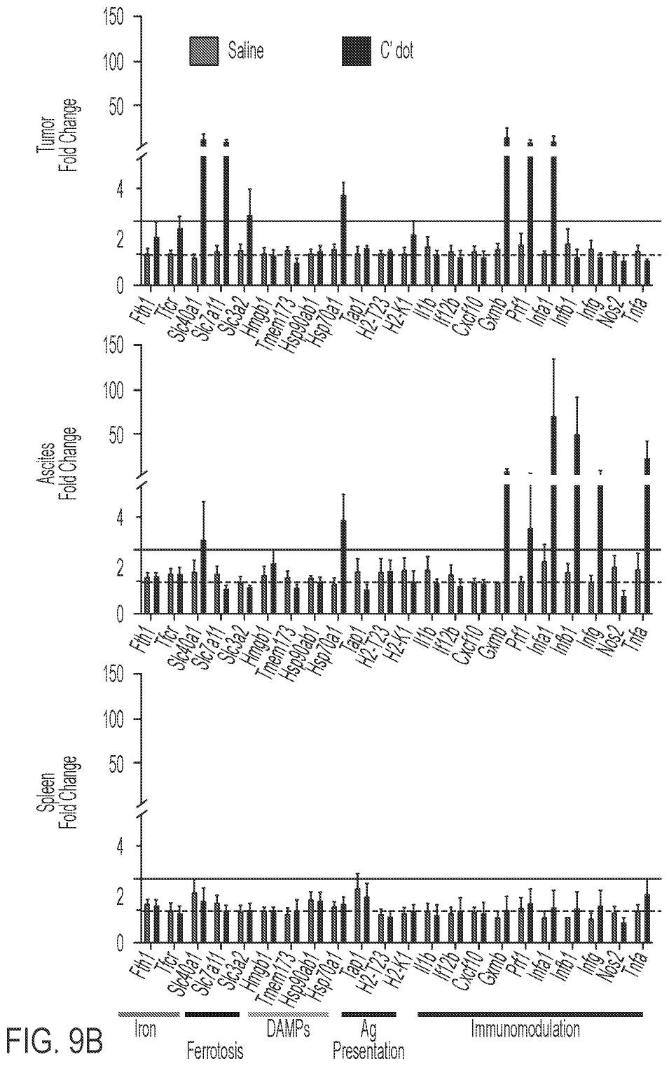

Using the described nanoparticle compositions from Example 1A, RT-qPCR can be performed to investigate alterations in the following multiple class-specific gene expression profiles as a function of particle concentration: (i) iron-related genes; (ii) ferroptosis; (iii) antioxidant response; (iv) damage associated molecular patterns (DAMPS); (v) major histocompatibility class (MHC) class-I antigen processing and presentation; and (vi) immune-related. In the described in vivo pilot studies (FIGS. 9A-9B), adult C57BL6 mice, inoculated with 1×10⁷ VEGF-A low ID8 cells, were i.v.-injected with three doses of C′ dots (12 nmoles/each). RT-qPCR of cell suspensions derived from harvested tumor tissues showed significant upregulation of multiple expression signatures (e.g., fold changes extending beyond horizontal yellow bars), including ferritin heavy chain, ferroportin, cysteine/glutamate antiporter system, DAMPs (heat shock proteins) and interferons (IFNα, IFNβ), as compared with vehicle controls. Interestingly, a similar trend was observed in the ascites, but not in the spleen, suggesting that the modulatory effects of C′ dots localized in target tissues. Gene expression profiling can be performed for tumor, ascites, and splenic tissue specimens to investigate modulation of expression signatures as a function of dose at both a local and systemic level. Tissue samples can be bisected for both mRNA isolation and cytokine release assays. Luminex bead-based multiplex assays can be used to quantify cytokine secretion, and to validate differential mRNA levels of immunostimulatory and immunosuppressive cytokines seen during expression analysis. Blood can be collected for cytokine and toxicity assays, and tumor and major organs/tissues (liver, spleen, blood, and bone marrow) harvested for pathology (H&E, IHC, microscopy). Mice can be euthanized for signs of distress, lethargy, or weight changes (greater than 15%).

C57BL6 female mice can be initially inoculated with ˜1×10⁷ ID8 cells i.p. and tumor burden monitored weekly by BLI. Single- or multi-dose (n=1, 3, or 6) i.v.-injections of C′ dots (12 nanomoles/dose; 60 μM stock) or saline vehicle can be administered to mice (n=7 mice/dose/particle) ˜35 days post-inoculation. If multi-dosing, C′ dots can be injected every 3 days until completion. Particle-treated and non-particle treated specimens can be harvested at 24 hours and at 1 week after the last injected dose; this can enable characterization of both innate (early) and adaptive (late) immune profiles. Similar to methods described herein, changes in tumor burden can also be monitored in situ and ex-vivo by BLI and fluorescence imaging. Gene expression profiling can be performed for tumor, ascites, and splenic tissue specimens to investigate modulation of expression signatures as a function of dose at both a local and systemic level. Tissue samples can be bisected for both mRNA isolation and cytokine release assays. Luminex bead-based multiplex assays can be used to quantify cytokine secretion, and to validate differential mRNA levels of immunostimulatory and immunosuppressive cytokines seen during expression analysis. Blood can be collected for cytokine and toxicity assays, tumor, and major tissues (liver, spleen, and bone marrow) harvested for pathology (e.g., H&E, IHC). Mice can be euthanized for signs of distress, lethargy, or weight changes (greater than 15%).

### Immunophenotyping and Transcriptomics of Tumor-Infiltrating Leukocytes.

To confirm that changes in gene expression translate into phenotypic changes in the TME, CD45⁺ tumor-infiltrating leukocytes (TILs) are isolated from tumor, ascites, and splenic tissues for immunophenotyping and single-cell RNA and T cell receptor sequencing (scRNA/TCR-seq) analysis. For immunophenotyping, a multiparametric flow cytometry (MPFC) panel is used, including those for NK-cells (CD11b⁺ NK1.1⁺), dendritic cells (DCs; CD11b⁺ CD11c⁺ MHCII⁺), M1 macrophages (CD11b⁺ MHCII⁺ Inos), M2 macrophages (CD11b⁺ MHCII⁺ CD206 Arginase1), cytotoxic T cells (CD3⁺ CD8⁺), Th1 cells (CD3⁺ CD4⁺ IFNγ Tbet⁺ CXCR3⁺), Th2 cells (CD3⁺ CD4⁺ GATA-3⁺ CCR4⁺), Tregs (CD3⁺ CD4⁺ CD25⁺ FoxP3⁺), monocytic myeloid derived suppressor cells (M-MDSC; CD11b⁺ Ly6G⁻ Ly6C^{high}) and granulocytic myeloid derived suppressor cells (G-MDSC; CD11b⁺ Ly6G⁺Ly6C^{low}). In addition, indicators of improved (i) effector T cell activation (increased CD8+/Treg), (ii) pro-inflammatory responses (increased Th1/Th2), and (iii) reduced immune suppression (increased M1/M2, decreased percentage of MDSC within tumors) to evaluate effective anti-tumor immune responses can be used. For scRNA/TCR-seq experiments, two additional cohorts of mice (n=7/cohort) can be treated with particles or saline vehicle using the dosing strategy that meets endpoint criteria for Example 1B, and specimens (e.g., tumor, spleen, ascites) harvested and processed for transcriptomics. CD45+ TILs can be isolated from specimens, and T cell receptor (TCR)-repertoire analyses performed.

Moreover, dose-dependent changes in treatment response, immune modulatory status, and cell death in orthotopic models using C′ dots can be monitored. Improved doses used to treat the ID8 orthotopic tumors can be applied to the UPK10 model, enabling us to investigate differential immunomodulatory effects and cytotoxicity across models. Further investigation of the extent to which treatment responses in particle-exposed tissues can reverse tumor-induced immune suppression by the successful treatment of mice inoculated with increasing numbers of luc+ VEGF-A high ID8 cells. This is expected to reduce the inhibition of CAR T cells and improve its effectiveness.

### In Vivo Biological Endpoints.

PEG-Cy5-C′ dot probes can be selected for combinatorial treatment studies using the following go/no-go criteria: (i) an increase in T cell activity, (ii) reverse immune suppression (e.g., a decrease in MDSCs, an increase Type I IFNs,); (iii) maximum tumor uptake % ID/g and minimum off-target effects.

In Example 1C, differential anti-tumor immune responses of CAR T cells in combination with C′ dots can be assessed to characterize dosing strategies needed to maximize efficacy and expand the window for CAR T cell therapy. For example, a timing window for CAR T cell administration in the UPK10 model can be determined. Limited efficacy of CAR T cell therapy in solid tumors has, in part, been attributed to an inhibitory TME that suppresses these cells and narrows the treatment window. It has been previously shown that ˜80% mice survive past 120 days when injected with 4H11-28z IL12-CAR T cells at 35 days post-inoculation of ID8 cells, this drops to ˜30% over the same time frame when injected at 42 days post inoculation. By treating ID8 tumors with C′ dots prior to CAR T cell injection, the therapeutic window may potentially be extended by reducing tumor-induced immune suppression and, in turn, increasing survival.

Anti-tumor cytotoxic effects of CAR T-cells for particle-exposed and non-exposed tumors can also be assessed. Without wishing to be bound to any theory, it is believed that C′ dots can prime/activate the TME to improve outcomes for CAR T cell therapies in solid tumors. To test combination therapeutic strategies and potential synergies, C′ dot administration can precede treatment with CAR T cells (FIG. 12).

### Treatment Strategies Combining CAR T Cells and C′ Dots.

Application of particle dosing strategies can be applied that maximized anti-tumor responses in ID8/UPK10 tumor-bearing mice to additional ID8/UPK10 tumor-bearing mice, with and without subsequent CAR T cell therapy (FIG. 12) for immunophenotyping and transcriptomics. Tumor, spleen, and ascites specimens can be harvested and analyzed for indicators (e.g., endpoints) of immune suppression and CAR T cell activation with immunophenotyping. Best results for each model meeting these endpoint criteria can be used to inform transcriptomics analyses.

### Survival Studies.

Treatment strategies leading to significant decreased in tumor growth can be repeated in this example to assess survival benefit for ovarian tumor-bearing cohorts.

### Induction of Ferroptosis for Improving Antigenic Heterogeneity in ID8 Tumors.

One major barrier to effective immunotherapy targeting is heterogeneity among cancer cells for the cell surface antigens that can be targeted by CAR T approaches, referred to as antigenic heterogeneity. Ferroptosis induction may enhance the effectiveness of CAR T cell therapy by inducing cell death that targets cells independently of their antigenic profile. As ferroptosis induced by C′ dots has also been shown to spread between cells, leading to the death of neighbors through a spreadable activity, this particular treatment may have potent activity to inhibit the suppressive effects of antigenic heterogeneity in cancers. Tumor tissues can be harvested ˜12 days from start of treatment. Four cohorts of mice (n=5/cohort) can be treated for each model: (i) combination C′ dots and CAR T cells (given after particles); (ii) C′ dots; (iii) CAR T cells; and (iv) vehicle. Transcriptomics can be repeated for TCR repertoire analysis, with and without ferroptotic induction.

### In Vivo TME Biological Endpoints.

The following endpoints apply: (i) Efficacy (+IL12 CAR T cells+C′ dots) greater than C′ dots alone, +IL12 CAR T cells alone; (ii) increased T cell activity, (iii) reverse immune suppression (e.g., decreased MDSCs, increased Type I IFNs).

### Example 2: Use of MC1-R Targeting C′ Dots, as Well as a Myeloid Cell-Targeting Pharmacologic Inhibitor, in Addition to Checkpoint Blockade (ICB)

While ICB has led to durable and, at times, curative treatment responses in subsets of patients harboring a range of malignant solid tumors, about 87% of patients do not derive long-term benefit, a result of the multiple mechanisms engaged by the tumor to evade the immune system. Among such mechanisms, myeloid cells are known to be a main driver of immune evasion and limited anti-tumor immunity. Importantly, high levels of myeloid cells in various tumors, such as melanoma and triple negative breast cancer (TNBC), herald a poor prognosis and promote immune suppression, which directly mediates resistance to ICB. In addition, tumor-associated myeloid cells contribute to the suppression of T cell function, which cannot be reversed with ICB. It has been discovered that sensitivity to ICB could be improved by selectively targeting the gamma isoform of phosphoinositide 3-kinase, PI3Kγ (a marker highly expressed by myeloid cells with a small molecule inhibitor (IPI-549), currently in clinical trials. New strategies, however, are critically needed to further improve response rates in ICB-resistant disease.

Moreover, although multiple immune suppressive mechanisms are known to mediate resistance in the melanoma TME, one important obstacle to improved immune recognition and control of tumors is the recruitment and presence of myeloid-derived suppressor cells or MDSCs, which play a pivotal role in limiting anti-tumor immunity. MDSCs may inhibit T cell responses in numerous ways, including but not limited to (i) degradation of amino acids (arginase, indolamine dioxygenase production), (ii) production of suppressive mediators (IL-10, TGF-β), (iii) production of adenosine, and (iv) expression of inhibitory receptors and ligands (PD-1, LAG-3, TIM-3, PD-L1).

Herein, non-targeting and C′ dots targeting melanocortin-1 receptor (MC1-R), overexpressed on melanoma cells, can be utilized as part of a combinatorial strategy to limit immune resistance and promote/sustain a more favorable TME. Nanoparticle compositions can be selected that maximize efficacy, cell death (e.g., ferroptosis), and proinflammatory responses in the TME with ICB. Described strategies can be extended to Example 1C to include PI3Kγ inhibition of myeloid cells in order to reverse immune suppression and limit resistance to ICB.

Moreover, the present disclosure describes that B16-GMCSF cells and tumors exposed to non-targeting C′ dots show that C′ dots can (i) serve as an agonist of interferon (IFN)-related pathways, (ii) upregulate antigen presentation pathways (e.g., TAP1, TAP2), and (iii) downregulate expression of immune checkpoint proteins (e.g., PD-L1). Collectively, findings described herein show that treatment with C′ dots can improve therapeutic efficacy by inducing pro-inflammatory phenotypes and directly enhance anti-tumor immune cell cytotoxicity, immunogenicity, and ferroptotic cell death.

### Example 2A. Identify Structural Properties of MC1-R Targeted and Non-Targeted C′ Dots that Maximize Immunogenicity and CD8 T Cell Cytotoxicity, as Well as Drive Induction of Ferroptosis and Other Cell Death Programs in Multiple Cancer Cell Lines

Experiments in this example seek to identify C′ dot candidates, both targeting and non-targeting, with characterized structural properties that can (i) activate and enhance cytotoxicity of CD8+ T cells; (ii) effectively polarize macrophages towards a pro-inflammatory phenotype, (iii) increase immunogenicity of cancer cells derived from melanoma (B16-F10, B16-GMCSF) and TNBC (4T1) models resistant to ICB (4T1 greater than B16-GMCSF greater than B16-F10); and (iv) maximize particle-driven cytotoxicity. For example, three tumor cell lines (one triple negative breast cancer line, 4T1, and 2 melanoma lines-B16-F10, B16-GMCSF) can be tested. When implanted in mice, these lines generate immunosuppressive TMEs and are immunoresistant to ICB in the following order: 4T1 greater than B16-GMCSF greater than B16-F10. Differential modulatory effects of C′ dots on macrophages, CD8+ T cells, and tumor cells can be assessed.

### Example 2A-1. Synthesis of New MC1-R-Targeting Alpha Melanocyte Stimulating Hormone (αMSH) Peptide-Linker Sequences for Particle Conjugation

Two second generation (2^{nd} gen) αMSH peptide-linker sequences (FIGS. 13A-13F) can be synthesized and characterized for particle conjugation to improve MC1-R binding affinity and enhance cellular uptake over the earlier prototype. The first construct replaces the current 12 carbon double (Ahx)₂ linker with a single 6-carbon Ahx linker, while the second construct replaces the Ahx linker with a PEG₃ linker. Both linker chemistries can be more hydrophilic than the original (Ahx)₂ linker, with the PEG linker matching the PEGylated C′ dot surface for improved display.

### Example 2B. Using Particles that Maximize Cytotoxic and Immunostimulatory Responses, Monitor Growth Inhibition and Survival, with and without ICB, Across Multiple Models

MC1-R-targeting C′ dots can be used for ferroptosis studies, with and without ICB. Treatment efficacy and the extent to which changes are modulated by immune cell activation, pro-inflammatory responses, and particle-induced cell death in the TME can be assessed.

### Example 2C. Determine Whether Resistance to Immune ICB can be Limited in Multiple Models by Combining Particle-Driven Cytotoxic Responses with Selective PI3Kγ Targeting to Subvert Immunosuppressive Components in the TME

Combinatorial dosing strategies using cytotoxic C′ dots, immune ICB, and a PI3Kγ-selective inhibitor targeting myeloid cells can be characterized to maximize efficacy, reduce immune suppression, and improve T cell effector function. This involves investigating different treatment and dosing strategies that combine particles, immune ICB, and PI3Kγ inhibitors to maximize tumor regression and reverse immune suppressive activities in the TME. This also involves investigating assay changes in myeloid-suppressive phenotypes that drive resistance, immune cell activation, and T cell-mediated cytotoxicity. Moreover, a myeloid cell-targeting agent—a selective PI3Kγ inhibitor (ICI-549, NCT02637531)—can be added to the described particle-driven combinatorial strategy with ICB, to selectively target immunosuppressive components in the TME and effectively limit immune resistance. IPI-549, currently in Phase 1 clinical trials (NCT02637531), alters the migration and production of inflammatory mediators, an effect that has been shown to reprogram the tumor-immune microenvironment and promotes cytotoxic (CD8+) T cell-mediated tumor regression. These studies can be built upon by adding C′ dots as a new combinatorial partner with both ICB and PI3Kγ inhibition to treat these same models. In addition to augmenting these proinflammatory responses, the use of C′ dots in these models lead to significant reductions in Tregs, inhibitory T cell receptors, and checkpoint proteins. Without wishing to be bound to any theory, it is believed that this combinatorial strategy can yield enhanced therapeutic responses and a new treatment paradigm for managing such immunoresistant models.

The described nanoparticle compositions screened using endpoints (below) and the characterized conditions from Example 2B can be used to further improve upon the described results in these immune-resistant models. Responses have been shown to synergize with ICB in the B16-F10 and B16-GM models, leading to significant improvements in immune resistance over that with ICB alone by increasing activated T cell and antigen-presenting cell populations, while decreasing inhibitory T cell-myeloid populations.

Example 2C can also investigate different treatment and dose schedules that combine C′ dots, immune ICB, and IPI-549 to maximize tumor regression and reverse suppressive activities in the TME. Treatment responses of C′ dots, combined with checkpoint blockade and IPI-549, can be examined in B16-F10, B16-GMCSF, and 4T1 models.

Assay Changes in Myeloid-Suppressive Phenotypes that Drive Resistance, Immune Cell Activation, and T Cell-Mediated Cytotoxicity.

Indicators of improved T cell activity (increased CD8+/Treg) and reduced immune suppression (decreased inhibitory receptors, decreased % of MDSC within tumors) have been used to evaluate effective anti-tumor immune responses.

### In Vivo TME Biological Endpoints.

C′ dot probes are evaluated for therapeutic effects alone, and in combination with ICB, with and without PI3Kγ inhibition, using endpoints that examine increased survival benefit: e.g., combination therapy >single agent therapy (e.g., ICB, C′ dots/αMSH-C′dots, PI3Kγ), increased T cell activity (CD8+/Treg), reductions in immune suppression/resistance on the basis of decreased myeloid cell populations (i.e., decreased MDSCs, tumor associated macrophages), macrophage phenotypic changes (i.e., decreased M2, increased M1), decreased inhibitory T cell receptors (i.e., LAG3, TIM3), increased Type I/II IFNs, and increased antigen presentation markers (i.e., MHC Class II). Through these endpoints, it can be understood how intrinsic therapeutic (“adjuvant”) properties of particle candidates can be leveraged within immunotherapeutic combination strategies to promote/sustain a more favorable TME, limit resistance, and improve efficacy in models with varying degrees of suppression. This requires detailed understanding of particle-driven and cellular/molecular mechanisms driving enhanced antitumor immunity.

### Example 2D. Assessment of Differential Modulatory Effects of C′ Dots on Immune Cells (e.g., Macrophages, CD8+ T Cells) and Tumor Cells Derived from Models Resistant to ICB for In Vivo Studies

Particles optimized for composition and structure and that exhibit the highest intrinsic therapeutic potential following systemic administration, including improved pro-inflammatory and cytotoxic responses, were used for in vivo studies across multiple cancer types.

One important obstacle to optimal immune recognition and control of tumors is the presence of suppressive myeloid cell populations, for example, MDSCs in the tumor microenvironment (TME). MDSCs may inhibit T cell responses in numerous ways. It was found that C′ dots polarize macrophages towards an M1 phenotype, in addition to increasing T cell activation and cell kill in the TME, in a manner that occurs independently of, for instance, ferroptosis induction. These findings suggest that in tumor microenvironments harboring a high proportion of tumor-associated myeloid cells, the ability to modulate myeloid-suppressive phenotypes towards a more pro-inflammatory one, while enhancing cytotoxicity, can potentially aid the reversal of tumor-induced immune suppression, reduce immune resistance to ICB, and, therefore, improve efficacy.

Experiments use syngeneic mouse melanoma models (e.g., 4T1, B16-F10, B16-GM) that demonstrate a spectrum of resistance to immune checkpoint blockade antibodies, mediated by the suppressive activity of infiltrating MDSCs, in order to examine the intrinsic therapeutic effects of systemically administered C′ dots. There is an association between resistance to immune checkpoint blockade (ICB) and myeloid cell infiltration in murine melanoma and triple negative breast cancer (e.g., 4T1) models. Compared with the myeloid cell-rich, immune resistant 4T1 model, the transplantable spontaneous melanoma model, B16-F10, showed less myeloid cell infiltration and activated CD8+ T cells in the TME; this model was only modestly responsive to checkpoint blockade (ICB). Relative to B16-F10 controls, however, the use of a genetically-engineered B16-GMCSF mouse model led to a more immunosuppressive TME, accompanied by loss of sensitivity to ICB. Thus, there are models with varying degrees of immunoresistance to ICB, and there is an association between resistance and myeloid cell infiltration (e.g., neutrophils, monocytes, macrophages, myeloid dendritic cells).

These studies allow for investigation of the contributions of particle-driven cytotoxic responses, macrophage polarization, and increased T cell activation, among others, to overall treatment response in a range of immune-resistant models. The combinatorial effects of C′ dots in the presence and absence of standard-of-care ICB, anti-PD1 and anti-CTLA-4 have been examined that can be combined with other experimental immunotherapeutic treatments, such as inhibitors of phosphoinositide-3-kinase gamma (PI3Kγ). The following describes a myeloid cell inhibitor to be used. IPI-549 (a selective inhibitor of PI3Kγ), is known to be highly expressed in myeloid cells, alters the migration and production of inflammatory mediators. In the absence of direct tumor cell targeting, it reprograms the tumor-immune microenvironment and promotes cytotoxic (CD8⁺) T cell-mediated tumor regression.

As discussed herein, C′ dots can drive cancer cell death processes, such as ferroptosis and immune-related cell death, to suppress tumor growth, potentially augmented by immunotherapies. Experiments can investigate how C′ dots can be systematically and rationally leveraged as combinatorial partners with immune ICB and myeloid-targeting PI3Kγ inhibitors (e.g., IPI-549) to improve treatment efficacy, as well as overcome immune resistance to ICB under conditions where tumors are infiltrated by high levels of suppressive myeloid cells.

The ability to modulate a suppressive phenotype toward a more inflammatory one represents a clear paradigm shift from traditional cancer nanomedicine drug delivery approaches that often deliver surface-conjugated cytotoxic agents or, more recently, cytokines or adjuvants, to enhance efficacy or improve immunotherapeutic responses, respectively. Experiments can evaluate new state-of-the-art combinatorial treatment strategies, tailored to the landscape of suppressive tumor-immune microenvironments, that combine multiple-dose regimens of renally-cleared C′ dots with standard and experimental immunotherapies in order to significantly reverse suppression and limit ICB resistance.

C′ dots can serve as specialized treatment tools, offering a distinct combination of multiple, separable anti-tumor intrinsic therapeutic activities in tumor models that (1) can modulate the TME toward a pro-inflammatory phenotype from one that is immunosuppressive, (2) increase anti-tumor immune cell activation and cytotoxicity in the TME, and (3) target cancer cells specifically for a potent form of cell death, ferroptosis, occurring through an iron-dependent mechanism. C′ dots also induce immune-related cytotoxic responses and can potentially induce other modes of cell death. In MC1-R expressing melanoma models, targeted MC1-R binding, uptake, and specificity can be maximized using new linkers of varying hydrophilicity for attachment of MC1-R targeting peptides to the particle surface.

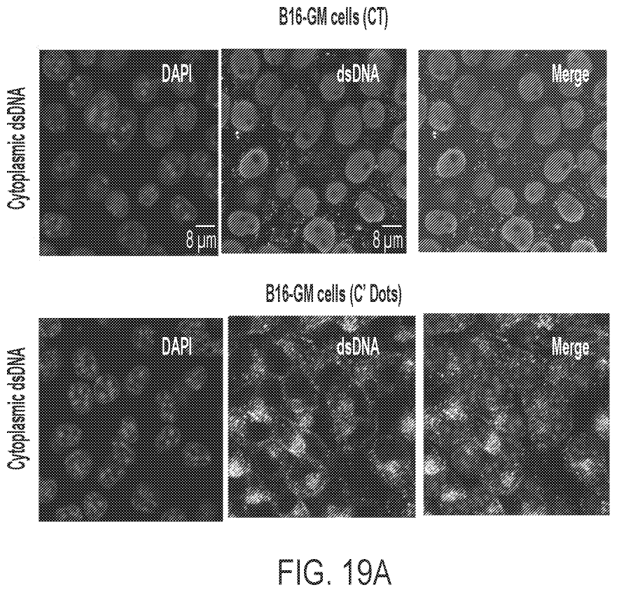

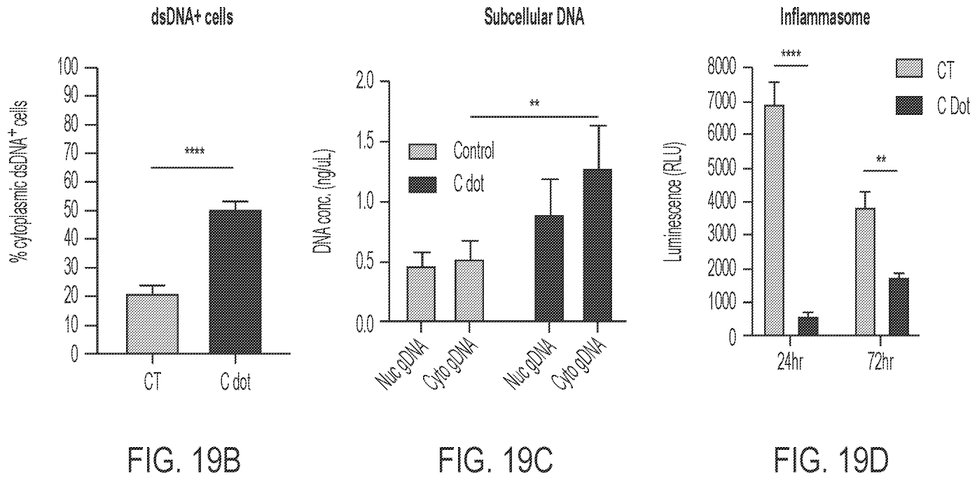

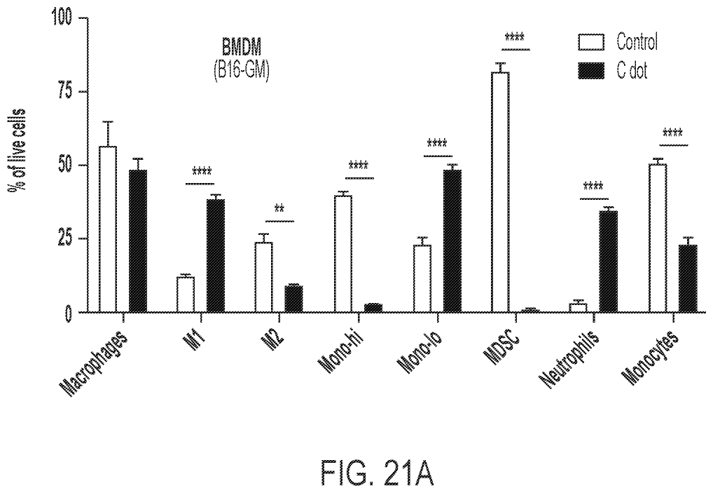

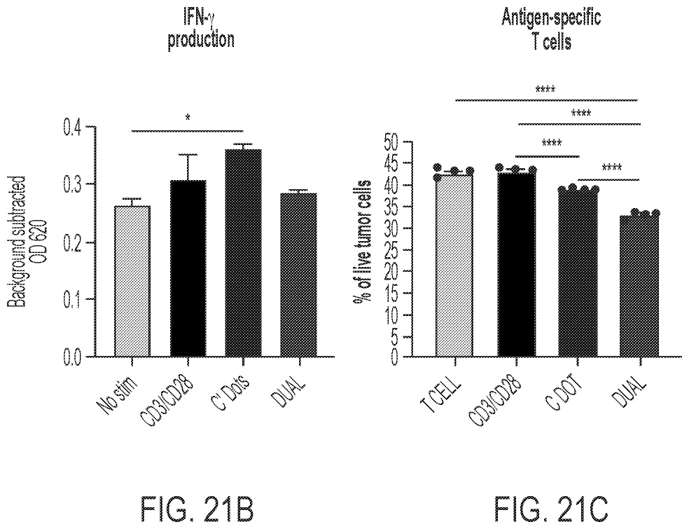

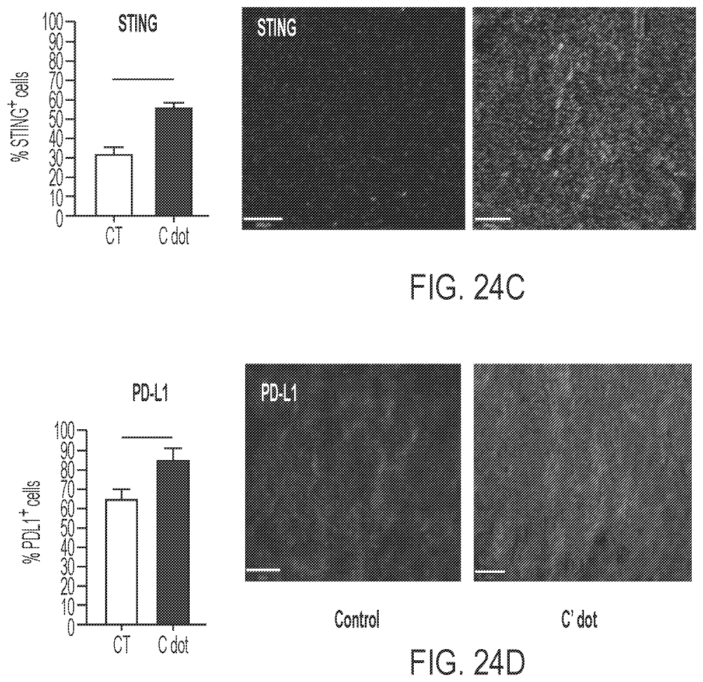

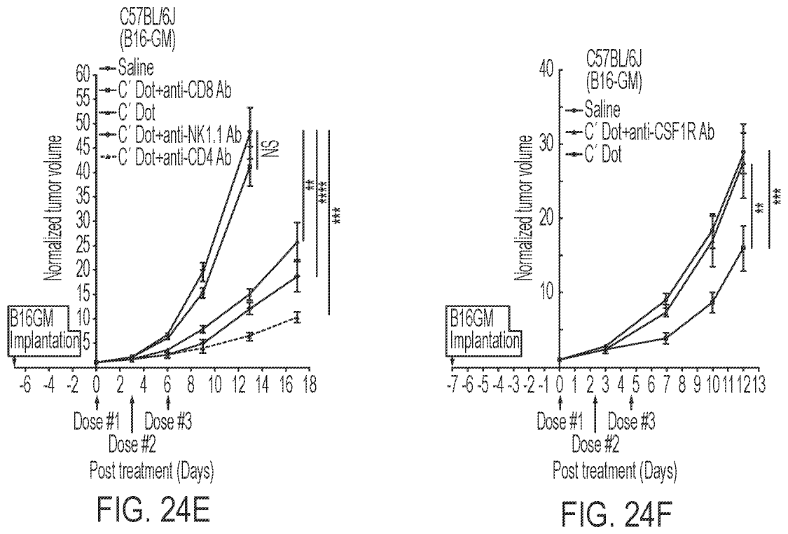

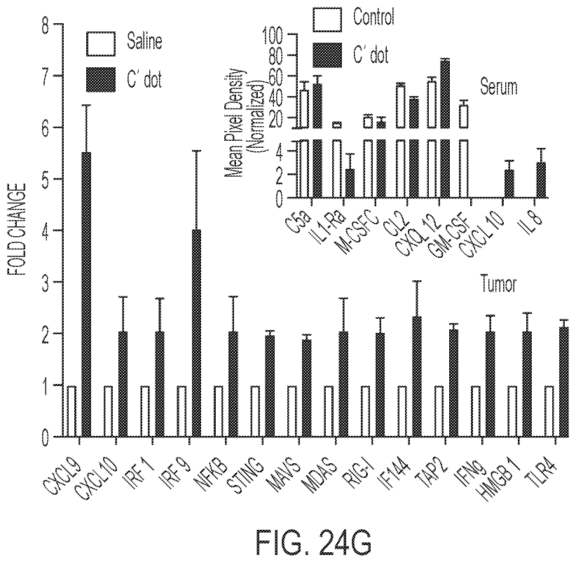

Particle candidates, optimized in terms of composition and structure, and that exhibit the highest intrinsic therapeutic potential, including improved pro-inflammatory and cytotoxic responses, are used to treat multiple cancer and immune cell types. Preliminary data of B16-GMCSF cells exposed to 15 μM C′ dots (72 h) demonstrated a significant increase in cytoplasmic double-stranded DNA (dsDNA) over controls by immunofluorescence (IF) (FIGS. 19A, 19B), confirmed using a fractionation protocol (FIG. 19C), and suggesting cGAS-STING activation. No contributions were found as a result of the inflammasome, noting marked reductions in caspase-1 from controls (72 h) (FIG. 19D) and in serum interleukin-1 receptor antagonist (Il-1R, see FIG. 24G) relative to controls. It was also observed polarization of BMDMs after C′ dot exposure (15 μM, 48 h) and near elimination of MDSCs (FIG. 21A). Lastly, particle-exposed (15 μM, 72 h) gp100+ T cells (from the pmel-1 transgenic model) showed an increase in IFNγ production as well as tumor-specific T cell kill when co-cultured with B16-GMCSF cells (blue bar, FIG. 21B, 21C), relative to both unstimulated T cells (gray bars) and co-stimulated (CD3/CD28) T cells (black bars). Moreover, C′ dots incubated with T cells co-stimulated with CD3/CD28 led to the highest T cell cytotoxic responses (red bar, FIG. 21C).

Based on these data, this example describes alterations in expression profiles, ROS, DAMPs (e.g., ATP), dsDNA, and immune cell phenotypes with both non-targeted and MC1-R-targeted C′ dot constructs, the latter improving cellular uptake. Particles can be incubated with B16-F10, B16-GMCSF, TNBC cells, T cells (splenocytes) and BMDMs from tumor-bearing C57BL6 mice. Cellular uptake, immune cell activation, cell death, and promotion of IFN responses can be assayed over a range of particle concentrations (100 nM-15.0 μM) and times (24-72 hours) using bulk RNA-seq to assess the effects of C′ dot constructs on various cell types and to elucidate mechanisms.

### Induction of Ferroptosis and Pro-Inflammatory Responses in Tumor-Bearing Models.

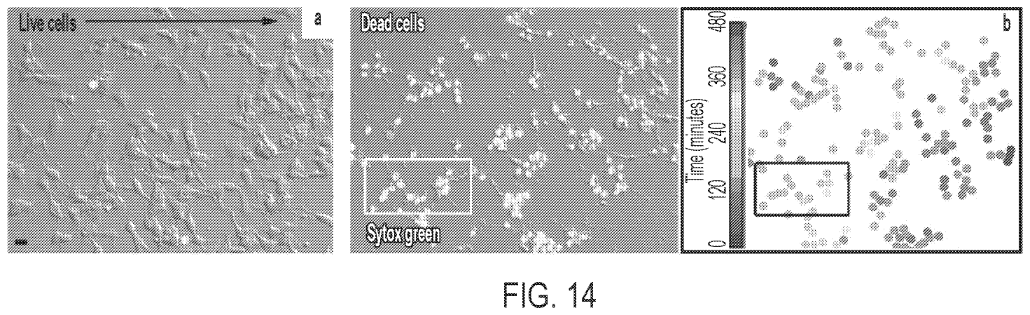

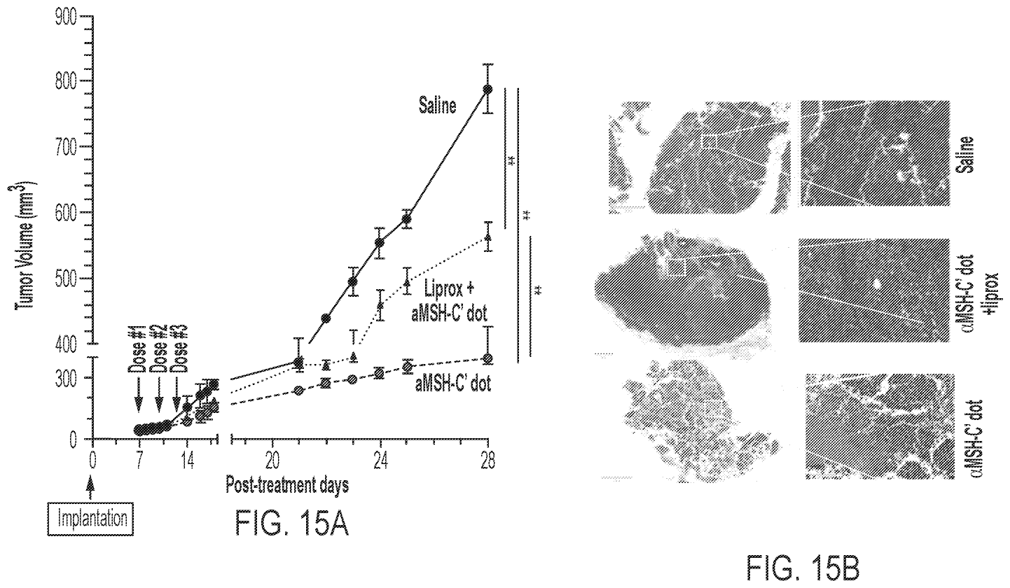

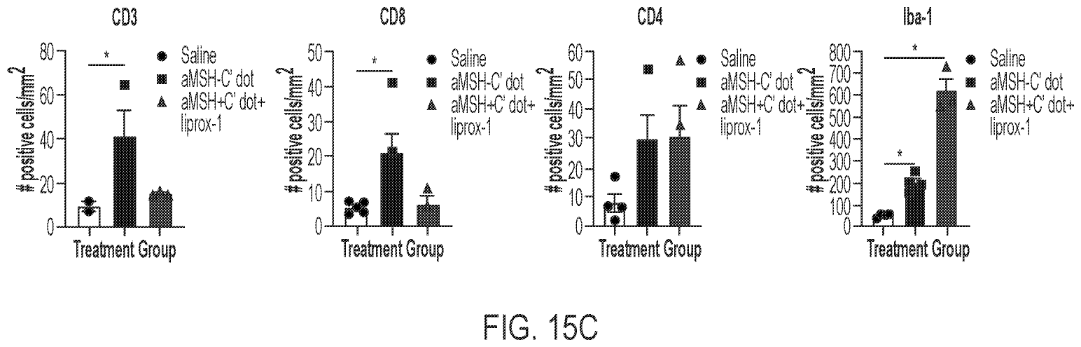

The present example describes a statistically significant and marked drop in tumor volume (red curve) to nearly 65% of control values (blue curve) following a multiple, high-dose (60 μM) regimen of i.v.-injected αMSH-PEG-C′ dots in B16-F10 mice when tumors reached 50 mm. After intraperitoneal (i.p.) injection of liproxstatin-1 (ferroptosis inhibitor), this effect is substantially attenuated (gray curve), confirming that ferroptotic induction significantly contributes to overall anti-tumor cell kill; the remainder is attributed to changes in the TME linked to immune-related cell death—an active area of investigation. H&E staining of C′ dot-treated tumor tissue shows marked tumor tissue necrosis that is reversed with liproxstatin-1, and is similar in appearance to control specimens (FIG. 15B). To evaluate the extent of immune cell infiltration, IHC was performed to assess for Iba1, CD3, CD4, and CD8 expression, with results graphically depicted in FIG. 15C. Statistically significant increases in the number of Iba1+ macrophages, CD3+ T cells, and CD8+ T cells per unit area were found in the melanoma TME after C′ dot treatment relative to control samples at about 23 days post-implantation. Interestingly, a rise in Iba1+ cells was seen in liproxstatin-treated tissues, probably reflecting an influx of macrophages to clear necrotic debris. Induction of ferroptosis in B16-F10 tumors is consistent with our prior results using other murine and human tumor types, even those not expressing MC1-R. These findings suggest that C′ dots deliver iron into cells, an activity that has been shown to lead to ferroptosis in culture (FIG. 14). These findings highlight the broad tumor-inhibiting capabilities of C′ dots that can be used to elucidate mechanisms underlying these effects.

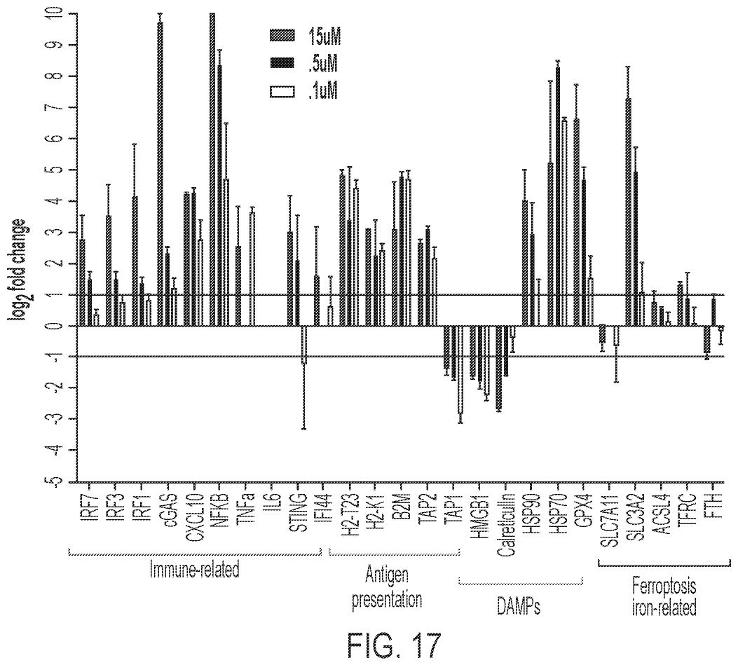

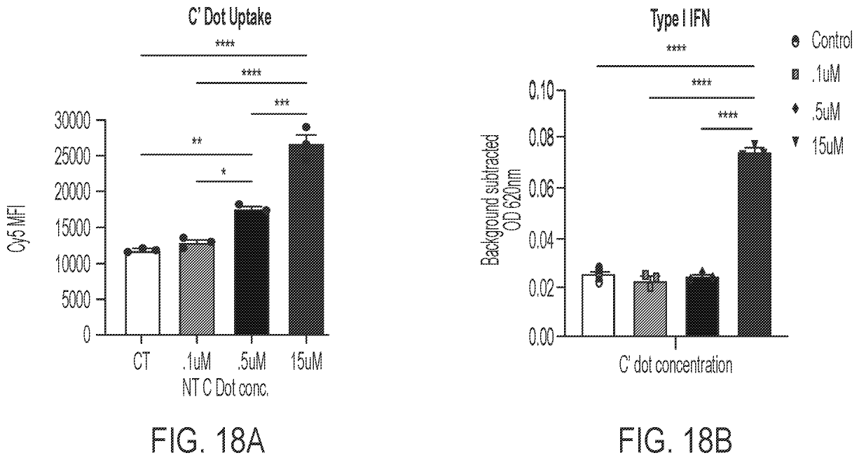

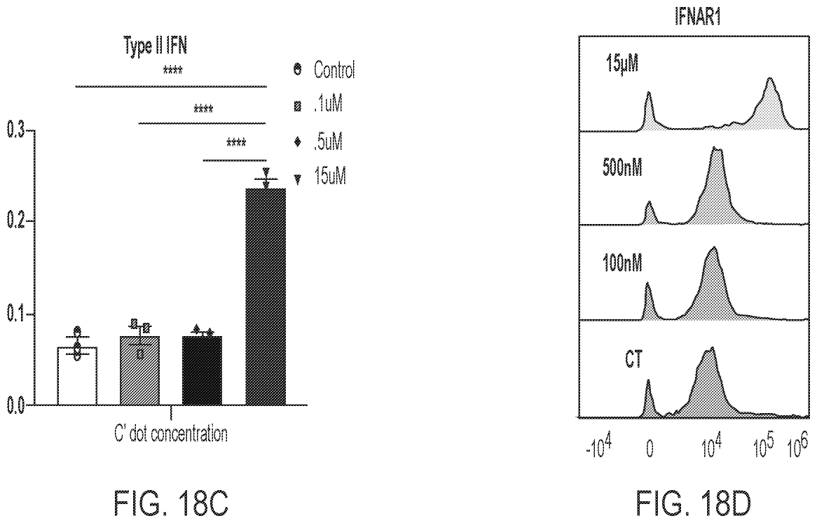

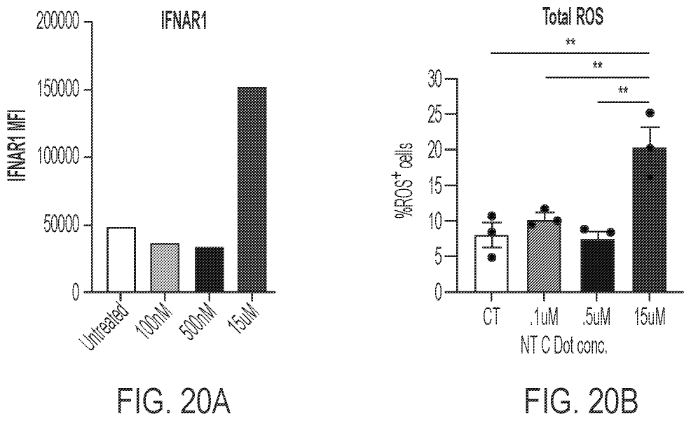

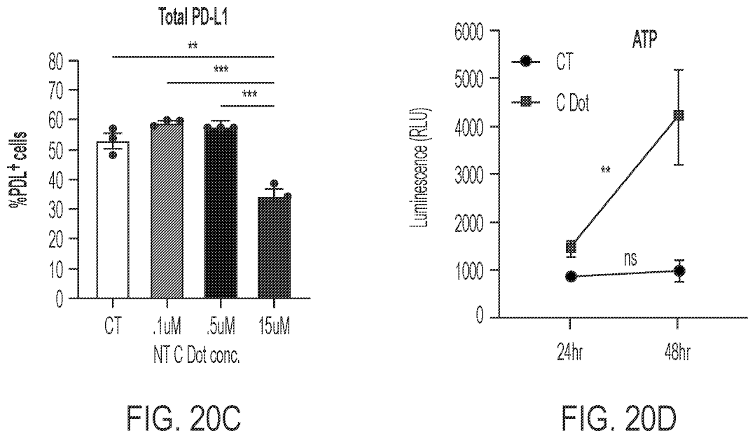

C′ dots upregulate iron-related genes. In addition, evidence of particularly pronounced immune signature gene changes in a well-characterized immunosuppressed model, B16-GMCSF, using gene expression profiling over a range of particle concentrations (100 nM-15 μM) has been found. Interestingly, B16-GMCSF cells treated with non-targeted C′ dots at 15 μM for 3 days led to significant upregulation (log 2-transformed fold changes extending beyond the horizontal green bars) of major histocompatibility complex class I (MHC-I) antigen processing and presentation genes, including TAP2 and H2-K1 (FIG. 17). In addition, DAMPs (e.g., HSP70/90) and immune-related genes, including interferon-stimulatory genes (ISGs), cGAS, STING, NF-KB, and interferon-response factors (IRFs) 1, 3, and 7, were also seen to be upregulated over the range of concentrations. These transcription factors are key drivers of tumor cell intrinsic immunity and lead to potent Type I IFN responses, the sequelae of known cell stress and cell death programs (FIG. 17). Mechanistically, it is therefore hypothesized that C′ dots induce a cell stress response in tumor cells, as evidenced by a significant increase in DAMPs, innate immunity, and oxidative stress. Previous studies have demonstrated a link between enhanced cellular stress/cell death programs with the sensing of nucleic acids and/or generation of oxidative stress from subcellular compartments (e.g., mitochondria) with engagement of the cGAS/STING pathway and initiation of Type I IFN responses. These proinflammatory responses are due, in part, to (i) an observed progressive rise in the uptake of non-targeted C′ dots in B16-GMCSF cells over 72 hours in a concentration-dependent manner (0-15 μM) by FC (FIG. 18A), which was accompanied by (ii) statistically significant increases in Type I/II IFN (FIGS. 18B-18C), Interferon Receptor 1 (IFNAR1) (FIGS. 18D-18E), and ROS (FIG. 20B), along with (iii) a significant decrease in PD-L1 expression (FIG. 20C). Moreover, a rise in ATP secretion was noted (FIG. 20D), consistent with cellular necrosis and immune-related death. However, relative to the B16-F10 model, C′ dot-exposed B16-GMCSF cells were found not to be as sensitive to ferroptosis, e.g., no upregulation of the majority of iron-/ferroptosis-related genes was observed (FIG. 17), nor was ferroptosis observed on time-lapse microscopy (data not shown).

Interestingly, B16-F10 melanoma cells treated with C′ dots at 15p M for 3 days in vitro upregulate major histocompatibility class (MHC)-I antigen processing and presentation and Type I interferon (IFNs) response genes. This increase in pro-inflammatory genes shows that C′ dots have the ability to enhance immunogenicity of B16-F10 melanoma cells and potentially combine favorably with different immunotherapies.

In summary, these results highlight the spectrum of anti-tumor inflammatory and cytotoxic changes induced by non-targeted C′ dots, which complement work described herein in which treatment of B16-F10 mice with low-dose αMSH-C′ dots (˜50 pmoles) led to an immune response characterized by a shift toward M1-activated macrophages, activation of natural killer (NK) cells, and infiltration of T cells and monocytes into tumors—without ferroptotic induction. Specifically, it was observed significant re-programming of expression signatures and immune cells driven by direct C′ dot uptake in B16-GMCSF cells, elucidating its multiple roles as an agonist of the cGAS-STING-NFκB, TLR-, and IFN-related pathways, and a modulator of immunogenicity and resistance (e.g., PD-L1 decrease). These and additional supporting data below underscore the potential of C′ dots to effectively prime endogenous innate and effector immune responses and induce cell death, providing strong mechanistic rationale for their synergistic combination with immunotherapeutics.

The present application describes dose- and time-dependent responses in B16-GMCSF cells treated with single-dose PEG-C′ dots. Ferroptosis-related genes, immune related genes, and antigen presentation/cell stress related genes are strongly modulated in response to PEG-C′ dot treatment. Specific components of “ferroptosis-related” pathways are significantly modulated in response to single-dose C′ dot treatment (e.g., GPX4, SLC3A2), whereas others are not appreciably altered (e.g., SLC7A11, Ferritin heavy chain). Moreover, the experiments show strong immunomodulatory effects due to PEG-C′ dot treatment at early and later time points.

In an immunosuppressive variant of the parental immunosilent murine melanoma line B16F10, in which the parental line was transduced with a vector promoting GM-CSF secretion (B16-GMCSF), it was found that a strong correlation between antioxidant response (GPX4) and the Stimulator of Interferon Genes (STING), in response to C′ dot treatment. Both GPX4 and STING were significantly upregulated in response to C′ dot treatment in vitro. This correlation is highly suggestive of two biologic phenomena: 1) The immunosuppressive variant B16-GMCSF contains a high capacity to resist antioxidant induced stress, and 2) A potential compensatory mechanism exists in which intracellular stress via C′ dot treatment triggers innate immune responses in an attempt to bypass this antioxidant stress resistant phenotype. Importantly, upstream and downstream components of STING activity/cell stress are also upregulated. Specifically, HSP70/90 and NF-kB, which ultimately led to increases in type I IFN mRNA levels. Likewise, components of the ferroptotic mechanism were upregulated. Namely the chaperone portion of the system Xc-antiporter, SLC3A2 and the transferrin receptor which is responsible for iron mediated endocytosis.

It was also found that multiple doses of αMSH-functionalized C′ dots (36 nmoles total, 60 μM stock), i.v.-injected into a genetically engineered mouse model of high grade glioma (mGBM), led to a reduced percentage of M2 or pro-tumoral macrophages within glioma by comparing immunofluorescence (IF) staining of untreated and treated tumor sections for M2 (CD206) and total macrophage (Iba1) expression markers. These findings were confirmed in a separate study of mGBM mice treated with low-dose particles (12 nmoles, 60 μM) versus vehicle control. At 96 hours p.i., M1-like (MHC-II^{high}Ly6C^{low}) tumor-associated macrophages increased in PEG-C′ dot-treated tumors relative to vehicle-treated and wild-type (WT) tumors, while M2-like (MHC-II-Ly6C^{low}) macrophages decreased relative to controls. T cell priming was also observed in a separate in vitro study when carboxyfluorescein succinimidyl ester (CFSE)-labeled CD8+ pmel-1 T cells, expressing gp100 (e.g., a melanoma-associated antigen), were co-cultured with particle-exposed bone marrow-derived antigen presenting cells (BM-APCs) loaded with gp100. Specifically, these T cells, derived from the pmel-1 mouse model, showed a significant increase in proliferation rate (CFSE) and activation state (CD44+CD25+). These data show re-programming of immune cells driven by direct C′ dot uptake, which occurs independently of ferroptosis.

In Example 2A, differential modulatory effects of C′ dots on macrophages, CD8+ T cells, and tumor cells derived from models resistant to ICB have undergone in vitro biological screening assays using αMSH-PEG-C′ dots and non-targeted C′ dots in order to identify candidates for in vivo studies. Particle candidates exhibiting the highest intrinsic therapeutic potential, including improved immunogenicity, antigenicity, and cytotoxicity, across multiple cancer and immune cell types, were be used to select nanoparticle compositions for in vivo studies. In addition, this part can examine whether alterations in expression profiles and immune cell phenotypes benefit from MC1-R-targeting C′ dots that can better localize within target cells and tissues. Particles were incubated with B16-F10, B16-GMCSF, TNBC cells, T cells (produced from splenocytes of naïve C57BL6 mice), and bone marrow-derived macrophages (BMDMs). Cellular internalization, immune cell priming, ferroptosis induction, and promotion of antigenic, antioxidant, and Type I IFN responses have been assayed over a range of particle concentrations (100 nM-15.0 μM) and exposure times (e.g., 24-72 hours).

### Melanoma and MC1-R-Targeting C′ Dots.

While the base particle possesses intrinsic therapeutic properties, modification of its surface chemistry, such as conjugation with αMSH peptides (FIGS. 13A-13E), can augment or attenuate these properties and other biological activities. Detailed structural investigations can be performed to assess whether improvements in C′ dot architecture and surface chemistry ultimately amplify intrinsic anti-tumor responses. These examples build on nanoparticles described previously, by the conjugation of newer generation cyclic α-melanocyte stimulating hormone (αMSH) peptide-linker conjugates (e.g., up to 20) to C′ dots for targeting MC1-R (FIG. 18A), as described below. MC1-R is a G protein-coupled transmembrane receptor overexpressed on melanoma cells and highly conserved in human and mouse. Recent data has shown that αMSH-PEG-C′ dots are phagocytosed by and accumulate within macrophages, which also express MC1-R, as well as B16-F10 cells. This is of interest given that monocyte-derived leukocytes (e.g., THP-1 cells) are known to express MC1-R, which it is hypothesized can be exploited to reduce suppressive myeloid populations while sparing T cell populations. Structure-activity experiments proposed herein can assess how reducing hydrophobicity of the linker between the αMSH peptide and C′ dot and/or replacing rhenium-cyclization by lactam cyclization (FIGS. 13B-13D) leads to enhanced peptide ligand surface display and receptor targeting given the up to 10-fold improvement in IC₅₀ values (FIGS. 13E-13F). Without wishing to be bound to any theory, it is hypothesized that the tuning of such specific C′ dot structural parameters (e.g., linker chemistry, ligand densities) can support and enhance intrinsic therapeutic activities, including cancer cell death as well as modulate innate and adaptive immune responses. Gene expression profiling can also be conducted to select particles that induce upregulation of gene expression signatures related to iron metabolism (e.g., FTH1, TF, SLC40A1), antioxidant enzymes (e.g., GPX4, system Xc⁻ cystine/glutamate antiporter), MHC class I antigen processing and presentation (e.g., TAP1, TAP2) and type I interferon response (e.g., IFNA1, IFNB1). Changes in gene expression can be measured using quantitative reverse transcriptase-polymerase chain reaction (RT-qPCR) with TaqMan assays in duplexed reactions with controls (e.g., GAPDH). Relative expression of M1/M2 macrophage polarization (e.g., TNFα, iNOS, CD86 vs. CD206, Arg1) and T cell activation (e.g., IFNγ) markers can be assessed in particle-exposed BMDMs. Higher expression of select non-secreted genes can be validated by western blot analysis.

### Co-Culture Studies: T Cell Proliferation and Cytokine Release Profiles.

Upregulation of gene expression profiles in melanoma cells leads to enhanced T cell activation. Incubation of melanoma tumor cell lines with C′ dots over a range of increasing concentrations (100 nM-15 μM) and incubation times (24-72 hours), prior to co-culture with T cells, can be performed. T cell activation can be assessed by cytokine release profiles (e.g., IFNγ, TNFα, IL-2, -10, -12, TGFβ, perforin, granzyme B) using Luminex assays and proliferation using cell proliferation tracking dyes (e.g., CFSE) and flow cytometry.

### In Vitro Biological Endpoints.

C′ dot probes have been selected for in vivo melanoma studies using the following endpoints (which can also be applied to TNBC cells): (i) cell viability less than 10% (ferroptosis); (ii) increased Type I/II IFN responses; (iii) increased immune-related genes (e.g., IRFs); (iv) increased IC₅₀ (αMSH-C′ dots) greater than IC₅₀ (peptide) (e.g., 10⁻⁹ μM), with internalization of the former.

In Example 2B, analysis of particle-driven cytotoxicity of the TME has been evaluated with and without immune ICB. Adult B16-F10, B16-GMCSF, or 4T1 tumor-bearing mice (♀ and ♂, 6-8 weeks old) were i.v.-injected with multiple doses of ferroptosis-inducing particles (αMSH-/PEG-C′ dots), as against 0.9% saline vehicle. This approach enables assessment of growth inhibition and survival across multiple models, as well as the observation of both acute/innate immune responses, as well as the more latent/adaptive immune responses in the absence and presence of ICB. ICB is another sensitizer of ferroptosis (e.g., through repression of the amino acid transporter, SLC7A11, which may synergize with C′ dots to enhance the efficacy of IT.

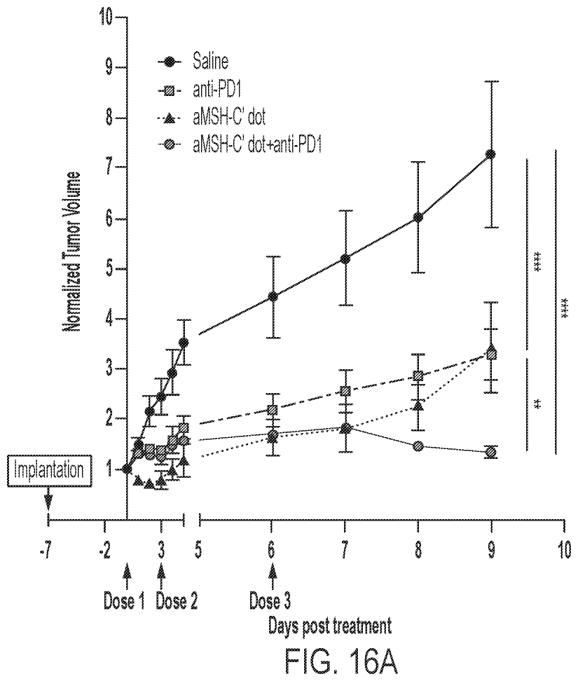

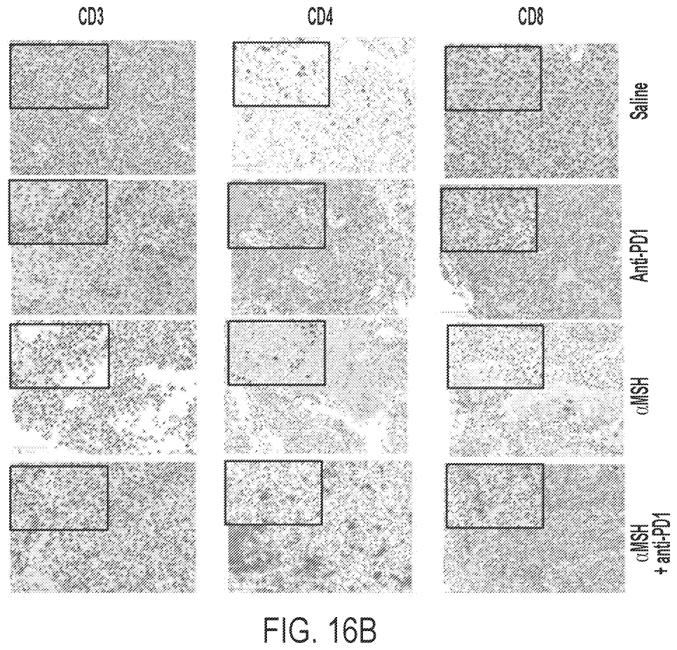

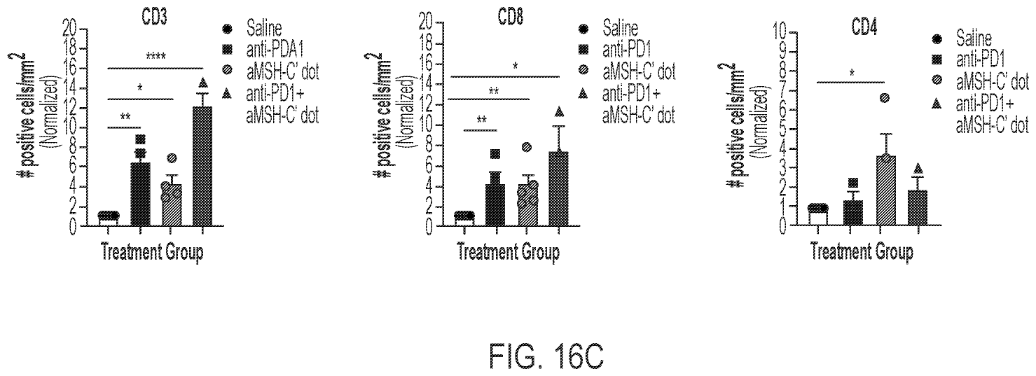

These studies also include correlation with transcriptomic profiles, immune modulatory status, and cell death. In particle-driven studies (FIGS. 16A, 16C), it was found that the combination of immune ICB with αMSH-PEG-C′ dots resulted in statistically significant B16-F10 tumor volume reductions compared with those using anti-PD1 inhibitor, αMSH-C′ dots, or vehicle control. Thus, C′ dots may potentially serve as synergistic partners with ICB to enhance treatment responses in immunosuppressive TMEs. Particle concentrations and incubation times found to significantly enhance particle-intrinsic therapeutic properties in vitro as initial starting values to induce ferroptosis in vivo—with and without multi-dose regimens of one or more checkpoint inhibitors, anti-PD-1 and anti-CTLA-4. Dosing strategies have been improved to maximize treatment efficacy, ferroptosis, and activation of the TME—all with the aim of improving immune suppression and attenuating immune resistance mechanisms. Tissues have been subsequently harvested for performing gene expression profiling and immunophenotyping; single cell suspensions from harvested tissues can undergo scRNA/TCR-seq. Results have been compared with untreated controls, and provide a landscape of mechanism-based changes to be further confirmed.

To investigate whether immune suppressive activities be reversed through particle-driven activities in the TME, preliminary data was generated using C′ dots in combination with ICB in the B16-F10 model (FIGS. 16A, 16C). It was found that 17 days after implantation (e.g., early time point relative to the 30-day interval in FIGS. 15A-15C), a statistically significant tumor volume reduction (e.g., nearly 90%) was observed in the cohort treated with C′ dots with anti-PD1 (relative to vehicle control), as against greater than 63% with particle alone (FIG. 16A). Analysis of correlative IHC data suggested that enhanced responses were due to increased lymphocyte infiltration, noting statistically significant increases in the number of CD3⁺ and CD8⁺ T cells per unit area for all treated cohorts, greatest for combination therapy (FIG. 16C).

In the B16-GMCSF model (FIGS. 24A-24G), a significant tumor volume reduction (˜60%) was seen in immunocompetent hosts (FIG. 24A), which was abolished in an immunodeficient mouse model (FIG. 24B) following triply dosed (60 μM, 200 μl/dose) i.v.-injected C′ dots when tumors reached 50 mm³. IHC of harvested tumor specimens showed STING and PD-L1 upregulation (green, FIGS. 24, 24D), the latter supporting addition of anti-PD1. To determine the extent to which the TME contributed to the findings of FIGS. 24A-24B, antibody depletion studies were further conducted using established protocols for anti-CD8/CD4/NK1.1 and anti-CSF1R antibodies (i.p.-injected). Results showed that CD8+ T cells and myeloid cells contributed significantly to particle-induced anti-tumor responses (FIGS. 24E, 24F). Further, gene expression and cytokine release profiles of tumor and serum (inset) samples (FIG. 24G) revealed significant activation of PRR pathways (e.g., STING, TLR, RIG-I), but no activation of the inflammasome (decrease in IL-1Ra) or production of suppressive GM-CSF (serum). Together, findings indicate that C′ dots can serve as a potentially synergistic partner with ICB to augment innate and adaptive immune responses in immunosuppressive TMEs. This example also describes mechanistic insights into the nature of the particle contributions driving anti-tumorigenic responses within suppressive TMEs.

Combination therapies were also evaluated in these models. Applicants have previously administered only immune checkpoint inhibitor therapies—anti-PD-1 and/or anti-CTLA4− to B16-F10, B16-GMCSF, and 4T1 tumors, with results showing only modest survival benefit over untreated controls for the more immunogenic B16-F10 model, while immunosuppressive phenotypes (B16-GMCSF, 4T1) showed no survival benefit. To overcome these limitations and examine anti-tumor responses in vivo—with and without multi-dose regimens of one or more checkpoint inhibitors, mice (6-8 weeks, female and male) were engrafted with B16-F10 and B16-GMCSF, and treated with combination C′ dots plus ICB, as against each agent alone and saline vehicle using particle concentrations and incubation times that were known to significantly enhance particle-intrinsic therapeutic properties in vitro. Dosing strategies were optimized to maximize treatment efficacy, ferroptosis, and activation of the TME—all with the ultimate aim of further reducing, if not eliminating, immune resistance mechanisms (e.g., via reversal of immune suppression). Tissues were also harvested for performing expression profiling and immunophenotyping; single cell suspensions can undergo scRNA/TCR-seq. Use of scRNA/TCR-seq can elucidate changes in intratumoral cell states and T cell clonality.

Preliminary data generated using C′ dots as a combinatorial partner with ICB in the B16-F10 model (FIGS. 16A, 16C), as described herein. It was found that, at 9 days after initial dosing (e.g., early time point relative to the 28-day interval in FIGS. 15A-15C), statistically significant tumor volume reductions were observed in all treated cohorts relative to vehicle control. However, the magnitude of the response was greatest with combination therapy, as against αMSH-C′ dots or anti-PD1 inhibitor monotherapies. These enhanced responses were due to increased lymphocyte infiltration; e.g., statistically significant increases in the number of CD3⁺ and CD8⁺ T cells per unit area, normalized to controls, for all treated cohorts, which was greatest for combination therapy, as shown by IHC (FIG. 16B). It was also noted significant elevations in CD4⁺ T cells, normalized to controls, for αMSH-C′ dot monotherapy, which was not seen with combination or anti-PD1 therapy; this remains an active area of investigation. These initial findings suggest that C′ dots can serve as a potentially synergistic partner with ICB to augment adaptive immune responses in immunosuppressive TMEs.

### Ferroptosis-Mediated Regulation of the TME.

Effects elicited within the TME have been assayed in a time-dependent manner, initially based upon the observed time of maximal particle uptake in tumors. Briefly, adult B16-F10, B16-GMCSF, or 4T1 tumor-bearing mice (female and male, 6-8 weeks old) were i.v.-injected with multiple doses of ferroptosis-inducing particles (αMSH-/PEG-C′ dots), as against 0.9% saline vehicle. Particles were allowed to circulate for extended time intervals, which included the observed times of maximal tumor uptake. Tissue specimens (e.g., tumor, liver, spleen, blood, and bone marrow) were harvested at these time points, as well as at earlier (e.g., t=0.5, 2, and 4 hr) and later times, extending out to about 2 weeks beyond the time at which expected peak particle uptake occurs (e.g., 24, 48, 72, 96, 168, 336 hrs; n=8 mice/time point). This approach enabled the observation of both acute and innate immune responses, as well as the more latent, adaptive immune responses in the absence of ICB. Harvested tumor tissue specimens were sectioned and CD45+ tumor-infiltrating leukocytes (TILs), as well as GFP+ tumor cells, and assayed for changes in markers: (i) gene signatures related to M1 and M2 macrophage polarization (RT-qPCR), (ii) cytokine release profiles (Luminex), (iii) cellular phenotypes (MPFC), (iv) iron metabolism, (v) antioxidant enzyme responses, and (vi) histopathology (H&E, IHC, confocal microscopy).

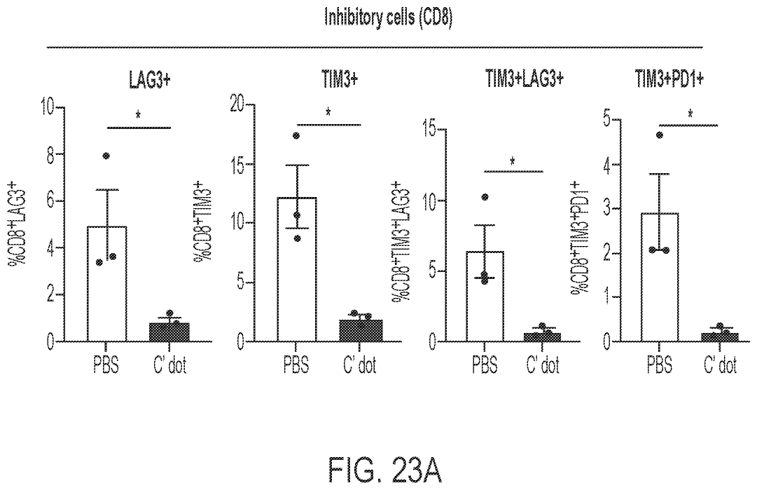

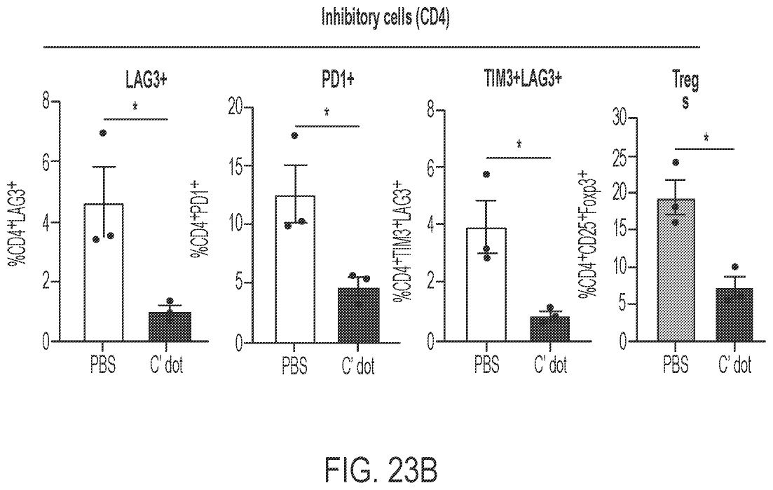

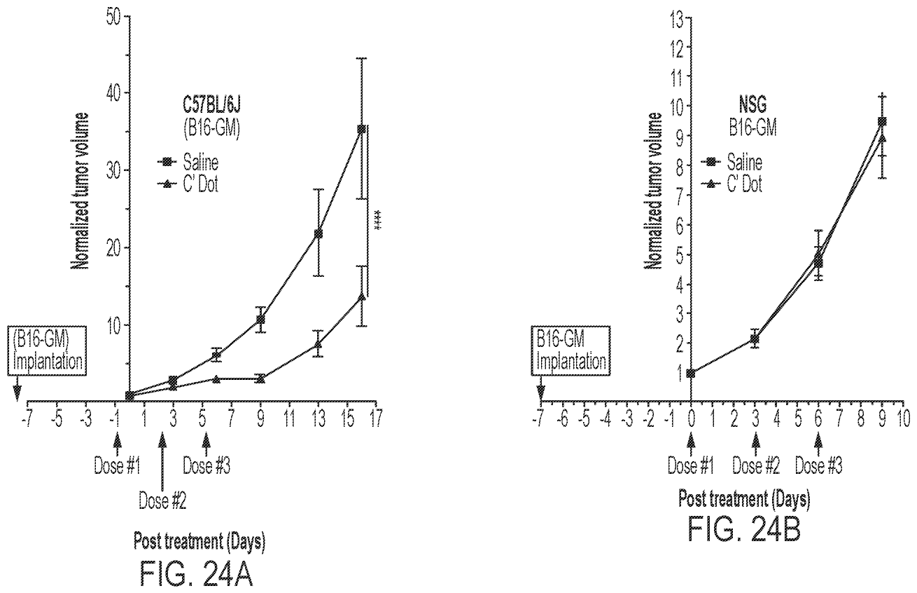

For example, preliminary data, acquired in tumor tissue specimens harvested from the B16-GMCSF model, 96 hours after three, high-dose non-targeted C′ dot injections, shows substantial increases in activated (anti-tumor) T cell and myeloid cell populations (FIGS. 22A, 22B), in addition to marked decreases in immune suppressive myeloid (FIG. 22C) and several inhibitory T cell populations (FIGS. 23A, 23B)—all statistically significant. Importantly, applicants do not observe any negative off-target effects in the serum (FIG. 24G, insert). Together, the foregoing changes suggest that C′ dots can substantially reduce TME suppressive mechanisms, meeting multiple endpoints (e.g., increase in CD8⁺/T_{reg}, decrease in MDSC) without off-target effects.

Immunophenotyping studies of CD45+ TILs were performed for all treatment groups using flow cytometry to quantify relative proportions of immune cells, including: (i) CD4+ Th1 cells (CD3⁺ CD4⁺ IFN-γ, Tbet⁺ CXCR3⁺), (ii) CD4⁺ Th2 cells (CD3⁺ CD4⁺ GATA-3 CCR4⁺), (iii) CD8⁺ T cells (CD3⁺ CD8⁺), (iv) Tregs (CD3⁺, CD4⁺, CD25⁺, FoxP3⁺), and (v) T helpers (CD3+, CD4+, FoxP3), (vi) M-MDSC (CD11b⁺ Ly6G⁻ Ly6C^{high}), (vii) G-MDSC (CD11b⁺ Ly6G⁺ Ly6C^{low}), (viii) M1 (CD11b⁺ MHCII⁺ Inos) and (ix) M2 (CD11b⁺ MHCII⁺ CD206 Arginase1). For each group, assessment of TMEs for increased T cell activity (CD8+/Treg) and for markers of improved immune suppression/resistance (e.g., decreased percentage of MDSCs, decreased inhibitory T cell receptors, increased Type I/II IFNs, and increased antigen presentation pathways) were conducted.

In addition, the extent to which observed changes in these immune TME parameters could also be attributed to a cell death process, e.g., immune-related cell death or ferroptosis, can be examined by repeating experiments in the presence and absence of the specific inhibitors, using the same methods above.

### Tumor Growth Inhibition in Immunosuppressed TMEs: C′ Dots Plus Checkpoint Blockade.

Alterations in the TME by C′ dots, in the presence of ICB, has be examined in B16-F10, B16-GMCSF and 4T1 models exhibiting a spectrum of immunosuppression. Particles have been either injected as multiple doses (n=3) alone or concomitantly with either the first three doses (“early”, on days 7, 10, and 13) or the final three doses (“late”, on days 16, 19, and 22) of anti-PD-1 (250 μg/mouse), anti-CTLA4 (100 μg/mouse), or anti-PD-1+anti-CTLA4 antibody regimens (see FIG. 12). Checkpoint inhibitors have been administered intraperitoneally (i.p.) on days 7, 10, 13, 16, 19, and 22 post-implantation of tumor cells for a total of 6 injections. Three doses of these inhibitors can occur either after or prior to particle administration. It can then be determined whether the order of administration of these agents is important in terms of modulating overall treatment responses.

Transcriptomics of Tumor-Infiltrating Leukocytes (TILs) that Drive Resistance, Immune Cell Activation, Proliferation, and T Cell-Mediated Cytotoxicity.

Using the treatment arm that shows maximum changes in gene expression, cytokine release, and one or more cell phenotypes for each lead particle, relative to vehicle controls, a parallel study for transcriptomics with scRNA/TCR-seq analysis in new cohorts of mice can be conducted. For example, a parallel study to analyze transcripts using scRNA/TCR-seq analysis in new cohorts of mice (n=6 total; 2/cohort per each lead particle and 2/cohort for saline vehicle) using the same experimental design can be performed. CD45+ TILs have be isolated from enzymatically digested tumor and splenic tissue specimens, along with GFP+ tumor cells, and can include TCR-repertoire analysis.

### In Vivo Biological Endpoints.

αMSH-PEG-Cy5-C′ dot and/or PEG-Cy5-C′ dot probes can be selected for combinatorial treatment studies in Example 2C using the following go/no-go criteria: (i) maximum tumor uptake/minimal off-target effects, (ii) increased survival benefit: combo (2 agents) greater than single agent therapy (e.g., ICB, C′ dots/αMSH-C′ dots), (iii) increased T cell activity (CD8⁺/Treg), (iv) reverse/limit immune suppression/resistance (decreased MDSC, decreased inhibitory T cell receptors, increased Type I/II IFNs, increased antigen presentation pathways).

Example 2D can examine alterations in expression profiles and immune cell phenotypes from MC1-R-targeting C′ dots that improve uptake within target cells and tissues. Particles can be incubated with B16-F10, B16-GMCSF, TNBC cells, T cells (produced from splenocytes of naïve C57BL6 mice), and bone marrow-derived macrophages (BMDMs). Cellular internalization, immune cell priming, ferroptosis induction, and promotion of antigenic, antioxidant, and Type I IFN responses can be assayed over a range of particle concentrations (100 nM-15.0 μM) and exposure times (24-72 hours).

### Gene Expression Profiling.

C′ dots have be incubated with B16-F10, B16-GMCSF and 4T1 cells over a range of increasing concentrations (100 nM-15 μM) and incubation times (24-72 hours) to select those that induce upregulation of gene expression signatures related to iron metabolism (e.g., Fth1, Tf, Slc40a1), ferroptosis (e.g., Slc7a11/Slc3a2 antiporter), antioxidant enzymes (e.g., Nrf2, Nqo1, Gpx4, Xc⁻ cystine/glutamate antiporter), MHC class I antigen processing and presentation (e.g., H2-D1, H2-T23, Tap1, Tap2) and Type I interferon response (e.g., Ifna2, Ifnb1). Changes in gene expression can be measured using quantitative reverse transcriptase-polymerase chain reaction (RT-qPCR) with TaqMan assays in duplexed reactions with controls (e.g., Gapdh). Results can be analyzed as a fold-change using the ΔΔCT method with log 2-transformation. Relative expression of M1/M2 macrophage polarization (e.g., Tnfa, Nos2, Cd86 vs. Cd206, Arg1) and T cell activation (e.g., Ifng) can be assessed in particle-exposed BMDMs and T cells, respectively. Higher expression of select genes can be validated by western blot analysis.

### Cytokine Release Assays.

Changes in gene expression profiles related to immunostimulatory or immunosuppressive cytokines, can be validated using a multiplexed cytokine release assay (Luminex). Samples can be snap-frozen and results can be quantified using a concentration standard curve and normalized based on a reference protein concentration sample.

### Co-Culture Studies: T Cell Proliferation and Cytokine Release Profiles.

To confirm that upregulation of gene expression profiles in melanoma cells, identified in these examples, leads to enhanced T cell activation, melanoma cell lines can be exposed to C′ dot candidates over increasing concentrations (100 nM-15 μM) and incubation times (24-72 hours) prior to co-culture with gp100 antigen-specific T cells from pmel-1 transgenic mice. T cell activation can be assessed by cytokine release profiles (e.g., IFNγ, TNFα, IL-2, -10, -12, TGFβ, perforin, granzyme B) using Luminex assays and proliferation using cell proliferation tracking dyes (e.g., CFSE) and flow cytometry.

C′ dot probes can be selected for in vivo studies (see Example 2) based on the following criteria in melanoma and TNBC cells: (i) cell viability less than 10% (ferroptosis); (ii) increasedType I/II IFN responses; (iii) increased immune-related genes (e.g., IRFs); (iv) increased IC₅₀ (αMSH-C′ dots) greater than IC₅₀ (peptide) (e.g., 10⁻⁹ μM), with internalization of the former.

### Example 3: Use of PSMA-C′ Dots and EB-RT, in Addition to Checkpoint Blockade

### Advanced Prostate Cancer.

Prostate cancer (PC) is the second most common cause of male cancer death in the US. In recent years, both the treatment management of PC and knowledge of mechanisms driving resistance to therapies targeting the androgen receptor (AR), a pathway in PC, have significantly improved. Current advanced PC treatment strategies involve administration of oral androgen deprivation therapies (ADT) or targeted AR signaling inhibitors, such as abiraterone acetate and enzalutamide, radiation therapy (e.g., EB-RT), and chemotherapy. Although gains have been made in understanding of AR biology and its signaling pathways, in turn, accelerating drug development efforts and leading to prolonged overall survival, these agents have, unfortunately, failed to halt the disease. In most patients, for instance, complete androgen blockade with ADT treatment results in remissions lasting 1-2 years, but cancer cells soon become resistant to ADT, concurrent with the emergence of metastatic castration resistant PC (mCRPC). Therefore, despite recent treatment advances, the disease eventually progresses, with survival often less than 20 months. These impediments have underscored the need for more efficacious treatments, resulting in a burgeoning number of radiotherapies (RT), including radium-223 for treating only symptomatic bone metastases, and those designed to target alpha- and beta-emitting radionuclides to PC via PSMA for treating bone and visceral disease.

### PSMA Radioligand Therapies.

PSMA radioligand therapies (or PRLT), used in clinical practice, emerged as a promising therapeutic option for PC about 2 decades ago. Used principally as a salvage therapy for mCPRC, ¹⁷⁷Lu-PSMA-617 therapy has shown a high response rate, while ²²Ac-labeled PSMA-617, developed more recently, has also shown remarkable results in naïve patients and patients that have failed previous treatments. Multiple studies using PRLT, however, have reported severe salivary gland (SG) toxicity. The largest database, compiled for 40 patients treated with ²²⁵Ac-PSMA-617, found frequent occurrence of severe xerostomia, which was dose-limiting, and the principal cause of patients discontinuing therapy or refusing additional doses. Another recent clinical trial reported that ˜23% of patients discontinued ²²⁵Ac-PSMA-617 treatment due to xerostomia. In addition, while reported SG effects for PRLT are of an acute nature, more chronic sequelae can occur in humans, such as renal toxicity due to high cumulated uptake at this site. Although antibodies have also been used as radiotherapeutic vehicles, there is an associated increased risk of developing dose-dependent marrow or hepatic toxicity, with limited tumor delivery and penetration. These adverse events have promoted efforts to develop effective preventive strategies for salivary gland (SG) toxicity.

### Tumor Microenvironment in PC.

Another hurdle to the treatment of PC is the tumor microenvironment (TME), which has proved to be a particularly challenging target, particularly for immunotherapies (ITs) due to their low mutational burden, lack of activated tumor-infiltrating lymphocytes, and specific genetic alterations that influence the immune landscape. Prostate tumors generally have an immunosuppressive microenvironment, including increased infiltration of M2-like macrophages, myeloid-derived suppressor cells (MDSCs), and suppressive dendritic cells (DCs), in addition to recruitment of regulatory T cells (Tregs) and increased levels of immunosuppressive cytokines. Moreover, about 50% of aggressive PCs often express high levels of PD-L1, which also suppresses the local immune response. As a result, studies supporting the activity of PD-1/PD-L1 inhibitors in PC have generally resulted in low responses to checkpoint blockade. However, more recently, the targeting of cytotoxic T lymphocyte-associated antigen 4 (CTLA-4) with ipilimumab in two phase 3 trials, although failing to demonstrate a survival benefit in mCRPC patients, showed significant durable responses in a subset of patients with favorable TME characteristics.

### Combination Radiotherapy and Immunotherapy.

The combination of external beam radiotherapy (or alpha-/beta-emitting peptide radioligands) and immunotherapy is an emerging and promising treatment paradigm. Radiation therapy (e.g., XRT) activates both the adaptive and innate immune systems through DNA damage responses and direct tumor cell kill, generating mutations in tumor-specific peptides (e.g., neoantigens), and promoting localized and systemic inflammatory responses that increase immune cell trafficking. Recent studies have highlighted both pro-immunogenic and immunosuppressive roles the immune system plays in the therapeutic effects of radiation in promoting tumor cell death. External beam radiotherapy (EB-RT), for instance, which has dominated preclinical combination radiotherapy-immunotherapy trials, has been found to upregulate MHC class-I, boost sensitization to cytotoxic lymphocytes, enhance tumor antigen presentation, increase generation of neoantigens and stimulate Type I/II interferon (IFN) production via activation of cGAS-STING responses—all which increase the anti-tumor effects of immunotherapy. Conversely, however, EB-RT, may lead to an increase in immunosuppressive cytokines (e.g., TGF-β) that decrease CD8 T-cell proliferation and stimulate CD4 T-cells to adopt a regulatory (Treg) phenotype. Moreover, depending on the tumor type, TME, and radiation dosing strategy, tumor-dependence on PD-1/PD-L1 immunosuppression may be enhanced in lesions responding to radiation, which requires the addition of a checkpoint inhibitor to avoid impeding radiotherapeutic-induced anti-tumor responses. It would be desirable, therefore, to utilize EB-RT or targeted radiotherapeutic ligands (e.g., peptides) with targeted particle compositions, which offers the possibility of reducing tumor-mediated immune suppression and dose-limiting toxicity while promoting anti-tumor cytotoxic responses. Targeted radiotherapeutic peptides have been used in combination with immunotherapeutic agents and are promising, showing survival benefit relative to the respective individual monotherapies in a PC model.

The present example describes advances to previous techniques. A first advancement is the development of a next generation, self-therapeutic sub 8-nm PSMA-targeted particle platform that, in combination with either ICB or EB-RT, enhances tumor cell kill in PC while, simultaneously amplifying proinflammatory responses and reducing immune suppressive activities. A second advancement is that these particle-based anti-tumor responses create a more favorable TME, which can be used to implement combinatorial therapeutic options for PC. A third advancement includes multiple self-therapeutic properties of the particle that can be exploited for anti-cancer therapy, including: (1) modulation of the TME by directly priming T cells, polarizing macrophages to M1 phenotypes, and altering cancer cell responses to ones that recruit and activate immune cells; and (2) induction of cancer cell death through an iron-dependent mechanism, ferroptosis. A fourth advancement is the use of improved newer-generation peptide linker chemistries, which optimize display of the PSMA targeting peptides on the particle's surface and improve target affinity by facilitating a tighter fit into the PSMA active site based on published crystallographic data.

In Example 3, PSMA-targeted particle probes are developed that overcome limitations of existing agents, synergize with checkpoint inhibitors, and activate the tumor microenvironment in prostate cancer (PC) to improve treatment outcomes. The combination of systemically administered PSMA-targeting C′ dots and ICB in transgenic PC models has been found to substantially reduce immune suppression, improve overall survival, and increase responsiveness to ICB without adverse off-target effects. These findings are due, in part, to improvements in PK and enhanced targeted tumor uptake over that found using non-targeted C′dots. Upregulation of immunosuppressive populations/markers in the spleen have not been observed. For these studies, a newer generation hybrid organic-inorganic ultrasmall (e.g., sub 10-nm) PSMA-targeting fluorescent core-shell silica nanoparticle, Cornell prime dots (C′ dots) was used that incorporated optimized linker chemistry for attaching the PSMA receptor pharmacophore to the core. The small size (enabling renal clearance) and well-controlled surface chemical properties of C′ dots led to favorable PK and therapeutic benefits (“target-or-clear” paradigm), as with earlier platforms. It is noted that use the same platform for studies that add EB-RT to systemic administration of PSMA-targeted C′ dots and ICB can be used; it is the insight of the preset disclosure that this combinatorial strategy can significantly improve upon findings without the addition target radiation.

An additional important benefit is that C′ dots are intrinsically therapeutic or “self-therapeutic”, contributing to anti-tumor responses. PC has generally been classified as a low mutation/tumor neoantigen burden neoplasm relatively unresponsive to IT. However, results from a recent clinical trial, conducted to determine if antigen specific T-cell responses could be elicited after immunotherapy in PC patients with low tumor mutational burden (TMB), demonstrated that patients did respond to immunotherapy if they had high intratumoral CD8+ T cell density, IFNγ-responsive gene signatures and/or antigen-specific T cell responses. High TMB was not necessary for neoantigen responses.

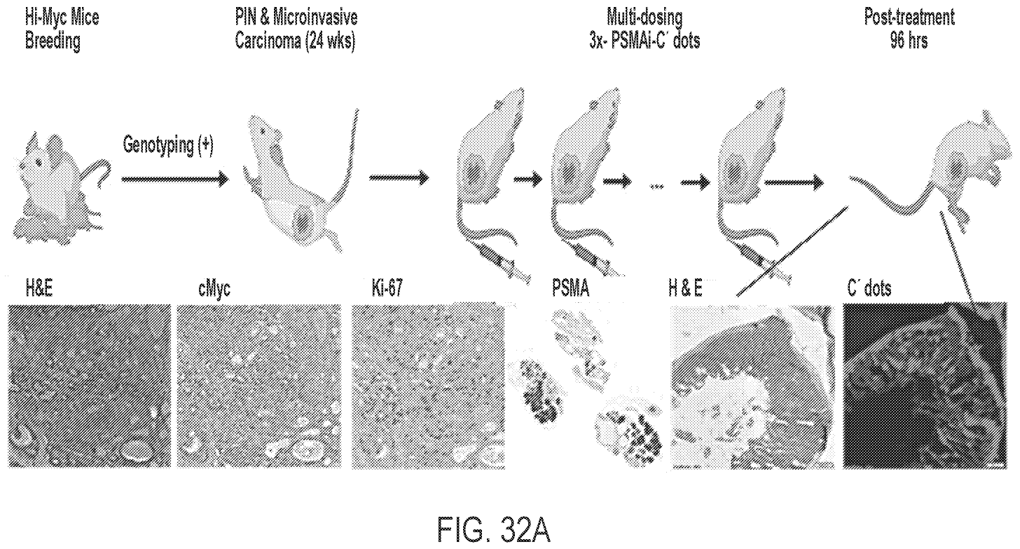

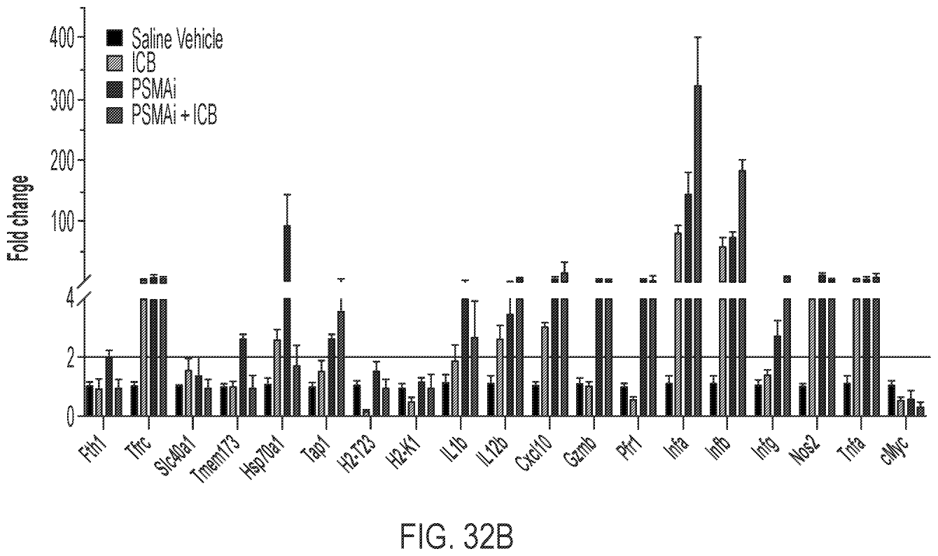

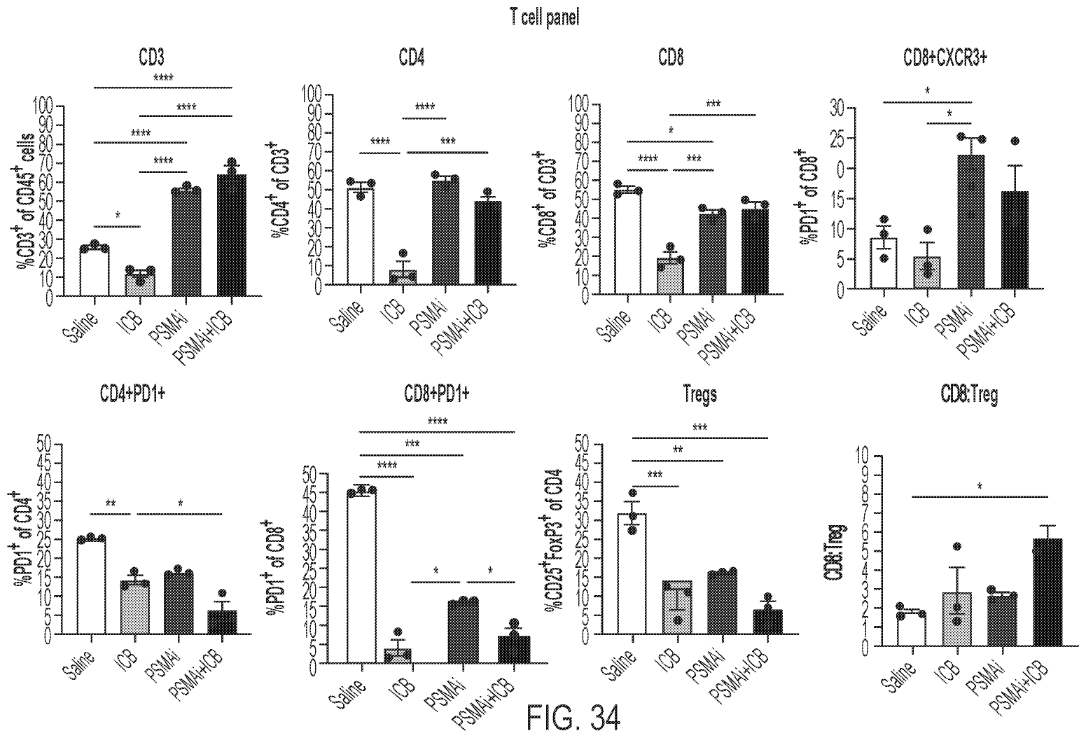

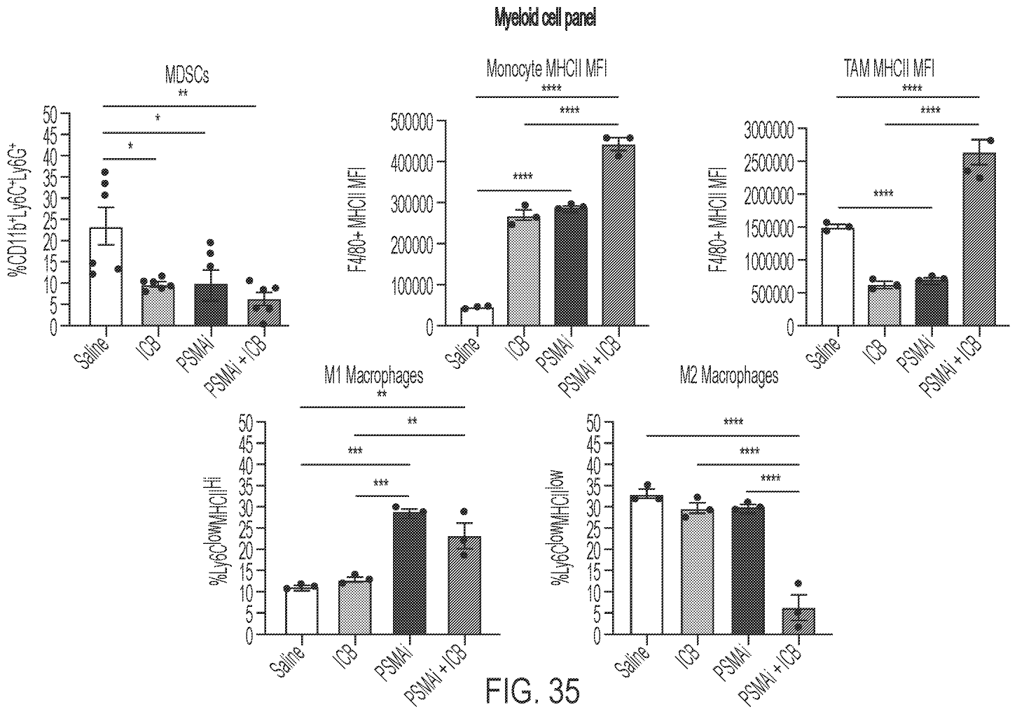

On the basis of the data described herein, the self-therapeutic properties of the particle create a more immunogenic TME that can synergize with EB-RT and prime the TME for the addition of IT. RT-qPCR results demonstrate upregulation of multiple gene expression signatures in the Hi-Myc model (FIGS. 32A-32B), including IFNs, Tmem173 gene encoding the STING protein, MHC Class I/II antigens, anti-tumor cytokines, DAMPS (Hsp90), and granzyme/perforin proteases, accompanied by decreases in several inhibitory T cell populations and increases in MHC-II expressing myeloid cell populations (FIGS. 34-35)—all activating the immune system and increasing anti-tumor activities.

By harnessing the intrinsic therapeutic properties of C′ dots, namely their ability to prime/activate effector cell populations, regulate macrophage phenotype, and induce an iron-driven cell death program, ferroptosis, it is further believed that the immune suppressive TME of PC can be re-programmed to potentially augment the efficacy of checkpoint blockade.

### Example 3A: Assess Re-Programming of the Immunosuppressive Prostate Cancer TME in the Hi-Myc Transgenic Mouse Model Using PSMA-Targeted C′ Dots

Experiments include evaluation of time-dependent particle-induced alterations of pro-inflammatory markers in the TME using gene expression profiling and performing immunophenotyping and transcriptomics characterization of tumor-infiltrating leukocytes derived from histologic specimens.

The experimental findings described herein suggest an improved mechanism for such combinatorial treatment strategies of prostate cancer (PC) whereby suppression of tumor growth through the use of PSMA-targeting particles in combination with EB-RT may be augmented due to the combination of the particle's intrinsic therapeutic properties with those resulting from radiation.

Presented herein is a PSMA-targeting C′ dot which, in and of itself, has the ability to remodel the TME to be less immunosuppressive and that, in combination with EB-RT and ICB, can improve PC treatment outcomes. Additional innovation is present in peptide linker chemistries, which improve display of the PSMA targeting peptides on the particle surface and improve target affinity by facilitating a tighter fit into the PSMA active site based on crystallographic data.

### Specific Tumor Targeting.

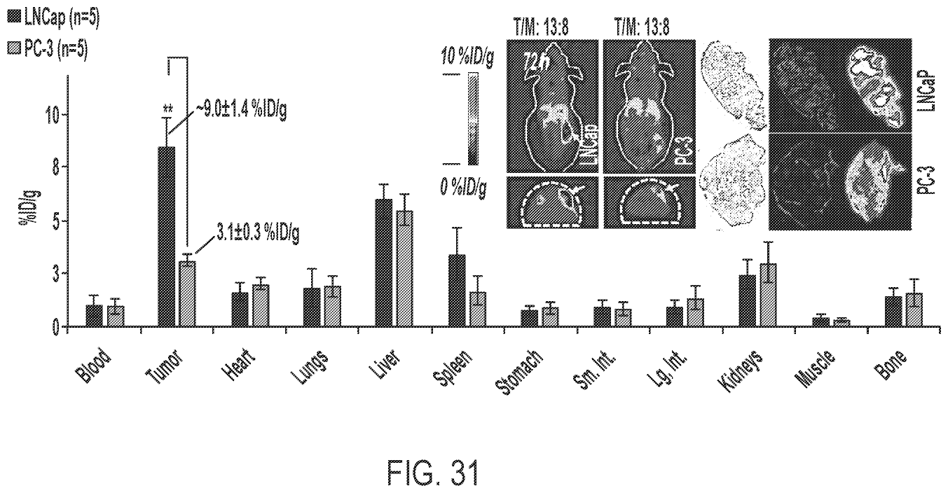

Target-specific uptake was evaluated in LNCaP and PC-3 xenograft models using ⁸⁹Zr-DFO-PSMAi-PEG-Cy5-C′ dots (FIG. 31) and PET imaging. A greater than 13-fold difference in the tumor target-to-background (T/B) ratio was found by PET for PSMA⁺ LNCaP xenografts (i.e, T/B˜14), as against PC-3 tumors (FIG. 31). Importantly, earlier tumor uptake values of the radiolabeled PSMA-targeting monoclonal antibody (mAb), J591, in PSMA⁺ LNCaP models are fairly equivalent to those found for PSMA-targeting C′ dots. Significantly greater absolute tumor uptake (% ID/g) was also noted for LNCaP versus PC-3 tumors. Ex vivo autoradiography and fluorescence microscopy at 72 h p.i. (FIG. 31) further confirmed specific uptake and target tissue penetration of ⁸⁹Zr-DFO-PSMAi-PEG-Cy5-C′ dots in LNCaP tumors. Radiostability was greater than 90% at 24 h p.i.

### DFO-PEG-Cy5-C′ Dot Impact on the TME.

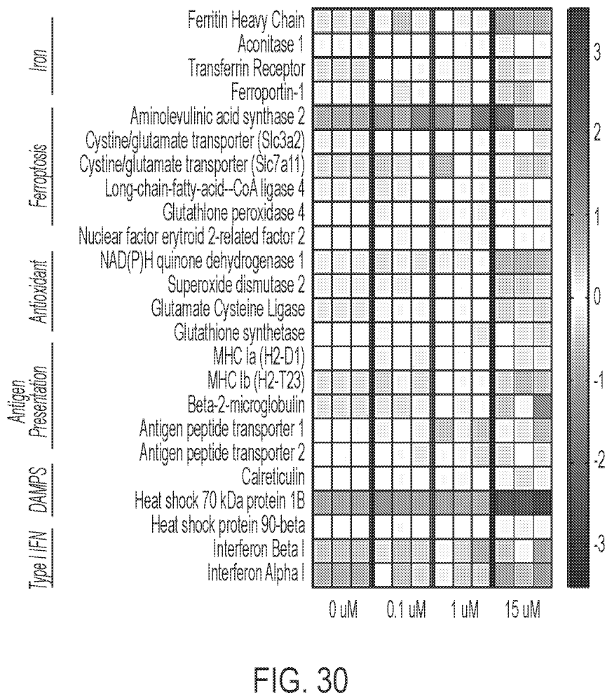

Cultured TRAMP-C2 cells were incubated with 0, 0.1, 1 and 15 μM DFO-PEG-Cy5-C′ dots (n=3) for 3 hours and examined using real-time quantitative polymerase chain reaction (RT-qPCR) for the expression of multiple gene signatures: damage associated molecular patterns (DAMPS), antigen presentation, Type I IFNs, ferroptosis, iron, and antioxidant genes (FIG. 30). Maximum upregulation of signatures across classes were seen at high C′ dot concentrations, including genes for ferritin heavy chain, antigen presentation, DAMPs/cell stress markers (heat shock protein 1B) and Type-I IFNs. Collectively these results indicate that C′ dots can induce upregulation of cellular stress and immune-related signatures. An in vivo pilot study of the TME in the Hi-Myc+ mouse model was also performed. Mice were bred, genotyped, and allowed to mature and at 24 weeks of age, Hi-Myc mice were administered a single particle dose (12 nmoles) of DFO-PSMAi-PEG-Cy5-C′ dots, and sacrificed 96 hours post-injection (p.i.) for histopathology and RT-qPCR analysis of the tumor tissue specimen.

### Example 3B: PSMAi Peptide-Linker Properties can Enhance Pharmacophore Interactions with the PSMA Target to Maximize PSMAi-PEG-Cy5-C′ Dot Binding Affinity, Avidity, and Target Accessibility in PC Cell Lines

To enhance pharmacophore interactions with the PSMA target, a PSMA-targeting peptide sequence, synthesized using standard solid phase methods, can be chemically adapted with improved linkers to preserve pharmacophore activity when conjugated via α,ω-heterobifunctional PEG ligands to NIR dye-encapsulating (e.g., Cy5)-C′ dots.

### Example 3B-1. Development of PEG-Cy5-C′ Dots Having Different Compositions and Surface Properties Using Peptide Constructs of Varying Hydrophilicity and DFO for PET Imaging

### Design and Synthesis of PSMAi Peptides.

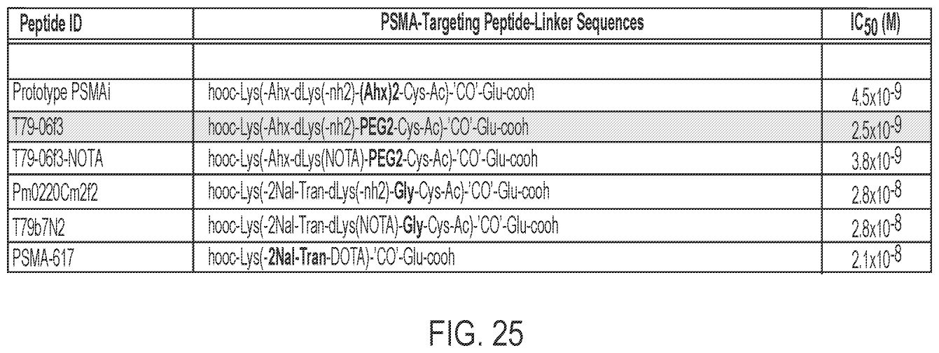

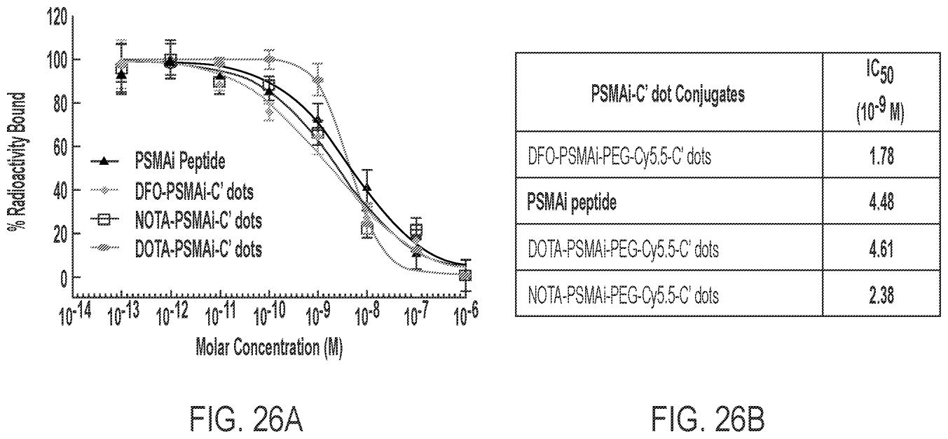

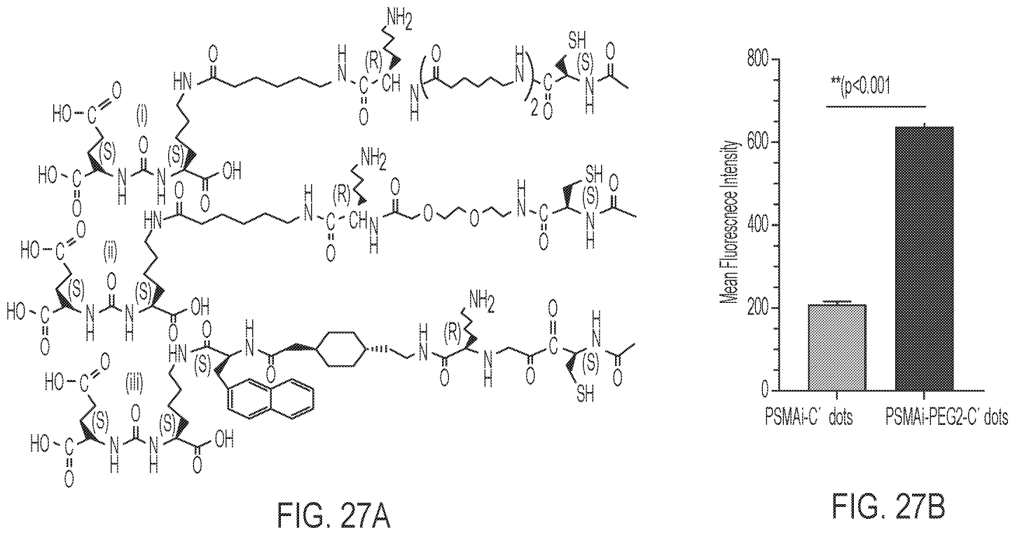

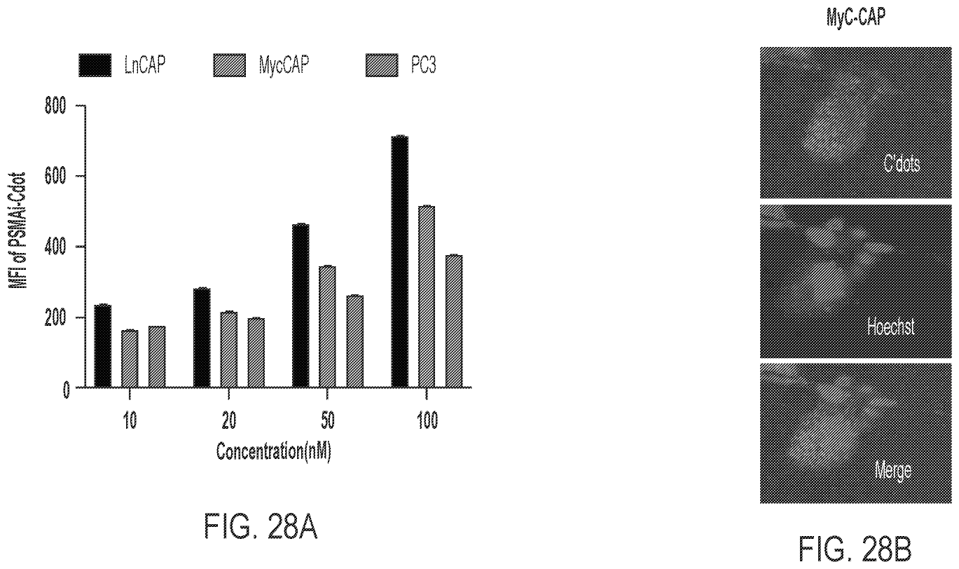

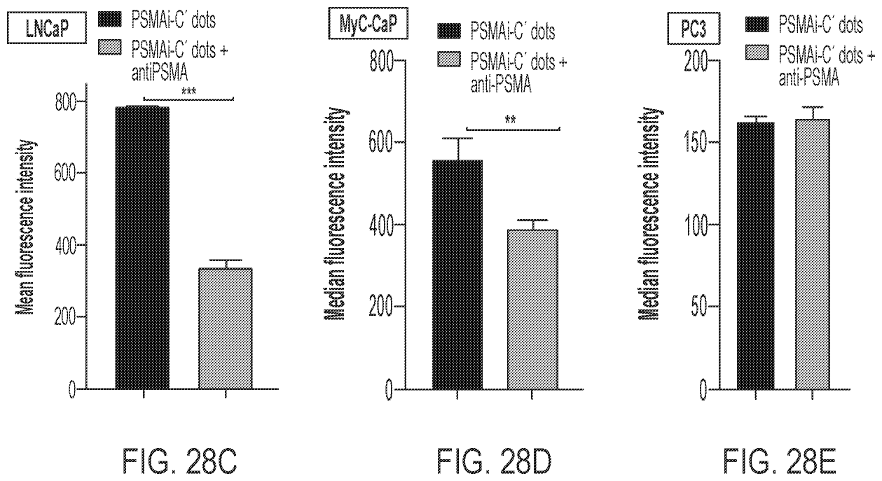

PSMAi peptides (FIG. 25) was produced using standard Fmoc solid phase peptide synthesis (SPPS) chemistry on a Tetras (Advanced ChemTech) multiple peptide synthesizer following the synthetic strategy used to produce the prototype PSMAi peptide (FIG. 27B.i). A limited number of PSMA avid peptides was examined for improved target affinity and tumor cell internalization and retention. The Glu-urea-Lys PSMA pharmacophore remained the same, while two linker modifications were examined. The first can substitute the hydrophobic (Ahx)₂ linker segment between dLys and Cys with a PEG2 linker (FIG. 27B.ii). A PSMAi-PEG2 linker should reduce hydrophobicity and allow improved pharmacophore presentation on the particle surface and enhance in vivo pharmacodynamics. The second linker modification can replace the (Ahx)₂ segment between pharmacophore Lys and dLys with a 2-Naphthyl-alanine (2Nal) tranexamic acid (Tran) linker (FIG. 27B.iii). Based on peptide-bound PSMA crystal structure analysis, the PSMAi-2Nal-Tran linker was designed to fill and interact with the larger diameter non-pharmacophore portion of the active site to improve PSMA affinity. Initial uptake data in LNCaP cells obtained for the prototype PSMAi-(Ahx)₂ and PSMAi-PEG2-Cy5-C′ dots (˜21 PSMA ligands per particle) demonstrated a factor of three improvement with the new PEG2 linker as against the prototype linker (FIG. 27C). Using the same new PEG2 linker construct, this example also showed concentration-dependent uptake for LNCaP, MyC-CaP, and PC-3 cells, with LNCaP demonstrating the highest mean fluorescence intensity (MFI) by FC (FIG. 28A). Internalization of (PSMAi-PEG2)-PEG-Cy5-C′ dots was confirmed by confocal microscopy in MyC-CaP cells after incubation with 100 nM particles for 4 hours (FIG. 28B). It was also showed that binding specificity by FC following exposure of LNCaP and MyC-CaP cells to this construct for 4 hours, and after pre-incubating cells with anti-PSMA antibody (FIGS. 28C-28E). Improvements in cellular uptake can be due to the number of ligands that are conjugated to the nanoparticle(s) (e.g., using the chemistries described herein) and/or other types of changes in surface chemistry (e.g., whether a positive or negative dye (e.g., Cy5) is used, location of dye incorporation (e.g., core vs. surface incorporation), or variations in shell size to core size ratio).

### ‘Structural’ Endpoints.

Structural ‘endpoints’ used as go/no-go screening criteria for this example are: (i) peptide, C′ dot purity >95%; (ii) C′ dot hydrodynamic diameter <8 nm; (iii) number of Cy5 dyes (>1); (iv) PSMAi ligands per C′ dot: 7 (min)-20 (max); (v) DFO ligands (3-5)/C′ dot.

### Competitive Binding Assays.

Prostate cancer cells have been incubated with increasing concentrations of PSMAi-PEG-Cy5-C′ dots bearing different numbers of peptides (e.g., ˜7-20) (with new/prototype linkers) for 4 hours to assess specificity. IC₅₀ values of chelated PSMAi-PEG-Cy5.5-C′dots (i.e., DFO-, NOTA- and DOTA) were determined in a competitive cell binding assay using the corresponding ⁶⁷Ga-labeled PSMAi peptide with human PSMA⁺ LNCaP cells (FIGS. 30A-30B). The apparent K_{d} values for the DFO-, NOTA, and DOTA-PSMAi-PEG-Cy5.5-C′ dot constructs were 1.78, 2.37, and 4.61 nanomolar (10⁻⁹ μM), about 2.5× (DFO) and 2× (NOTA), and greater than the peptide alone (4.47×10⁻⁹) or equivalent (DOTA).

### Gene Expression Profiling.

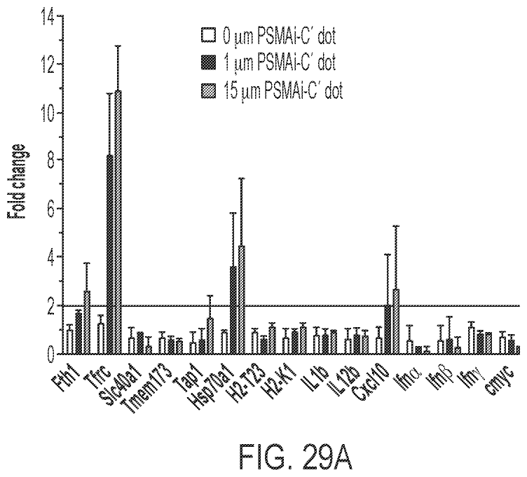

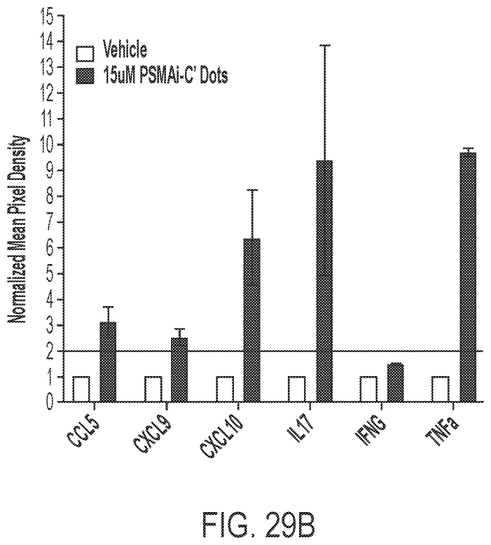

Non-targeting and PSMA-targeting C′ dots (from this example) can be incubated with MyC-CaP (derived from the Hi-Myc model) and TRAMP-C2 cells over a range of concentrations (100 nM-15 μM) and incubation times (24-72 hours) to select candidates upregulating transcription related to antigen presentation (e.g., H2-K1), Type I interferon (e.g. Ifnb1), damage-associated molecular patterns (e.g., Hsp70a1), iron metabolism (e.g., Tfrc, Fth1), and immunostimulatory or immunosuppressive cytokines (e.g., Il1b, Il10). Transcription changes can be measured by quantitative reverse transcriptase-polymerase chain reaction (RT-qPCR) with TaqMan assays in duplexed reactions with controls (e.g., GAPDH). Results can be analyzed as fold changes using the ΔΔCT method with the log 2-transform. Preliminary data from particle- (versus vehicle-) treated MyC-CaP cells show concentration-dependent upregulation of iron-related genes and DAMPs (FIG. 29A), as well as pro-inflammatory cytokines/chemokines (FIG. 29B).

### Cytokine Release Assays.

Changes in gene expression profiles related to immunostimulatory or immunosuppressive cytokines described here, were validated using targeted cytokine proteome assays (FIG. 29B). Samples were quantified using a concentration standard curve and normalized based on a reference protein concentration sample.

### Specific Tumor Targeting.

Target-specific uptake was also evaluated in LNCaP and PC3 xenograft models using ⁸⁹Zr-DFO-PSMAi-PEG-Cy5-C′ dots and PET imaging. A greater than 13-fold difference in the tumor contrast or tumor-to-background (T/B) ratio was found by PET imaging for PSMA⁺ LNCaP xenografts (e.g., T/B˜14), as against PC3 tumors. Importantly, earlier tumor uptake values of the radiolabeled PSMA-targeting monoclonal antibody (mAb), J591, in PSMA⁺ LNCaP models were fairly equivalent to those for PSMA-targeting C′ dots. However, the PK of C′ dots are more favorable than that for mAbs. Antibodies have blood clearance half-times of days to about a week versus ˜15-20 hours for C′ dots. Favorable C′ dot PK can reduce off-target effects (i.e., RES) compared with mAbs. Further, the smaller size (˜6 nm) and essentially neutral charge of C′ dots enable better tumor tissue penetration/distribution within target tissues relative to mAbs.

### In Vitro Biological Endpoints.

C′ dot probes were selected for in vivo studies using the following criteria for prostate cell studies: (i) IC₅₀ value (PSMAi-C′ dots)<5 nM with cellular internalization by FC and live cell imaging; (ii) specificity (block)≥50%; and (iii) specific activity (particle tracer)>1×10⁴ Ci/mol.

### Example 3C: Evaluate Therapeutic Efficacy in the Hi-Myc Transgenic PC Model Using PSMA-PEG-Cy5-C′ Dots Alone and in Combination with EB-RT and Correlate Findings with Tumor Volumes

An immune-competent PSMA⁺ Hi-Myc (FVB-Tg(ARR2/Pbsn-MYC)7Key/Nci) mouse model, which develops pre-neoplastic prostate intraepithelial neoplasia (PIN) in ˜2-4 weeks and imageable invasive carcinoma at >6 months is used for all in vivo studies (FIGS. 32A-32B). Growth inhibition and survival will be assessed in the Hi-Myc transgenic PC model, an immunocompetent model which recapitulates the molecular features of human PC in addition to expressing high PSMA levels. Efficacy can be evaluated using combinatorial therapies (e.g., PSMA-targeting C′ dots+single-dose EB-RT versus non-targeting C′ dots+single-dose EB-RT) or single-agent therapy (i.e., PSMA-targeting C′ dots, non-targeting C′ dots, EB-RT) can be evaluated, as against controls, correlating responses with initial tumor volumes and tumor growth volume reductions post-treatment using 7T MR imaging. Initial tumor volumes can range from about 8-10 mm³ to 150-200 mm³; larger tumor volumes would result in greater morbidity.

### Example 3D: Identify Structural Variations in DFO-PSMAi-PEG-Cy5-C′ Dot Tracer Surface Chemistries that Lead to Favorable Biodistribution and PET Imaging Profiles

Based on screening assays, PK and tumor-targeting studies can be performed using candidate PSMA-targeting particle probes in the PSMA-expressing Hi-Myc mouse model.

### Example 3E: Assess Survival Benefit and Safety of EB-RT in Combination with PSMA-Targeted Versus Non-Targeted C′ Dots with and without ICB in the Hi-Myc Model

To determine whether further improvements in survival can be achieved over EB-RT plus PSMA-targeted (versus non-targeted C′ dots), combination therapy studies in the PSMA⁺ Hi-Myc model can be performed using EB-RT, followed by PSMA-targeted C′ dots and dual ICB (anti-PD1+anti-CTLA4 mAb). Responses can be compared with EB-RT plus non-targeted C′ dots plus dual ICB, EB-RT alone, particles alone, dual ICB alone, and vehicle control. Irradiating tumor tissue with EB-RT can produce reactive tumor antigens or mutations that can then be recognized and destroyed by the host immune system, augmented by the addition of self-therapeutic C′ dots and dual ICB.

In vivo biological endpoints for all treatment studies and subsequent TME analyses are based on the following in vivo criteria: (i) survival benefit: EB-RT+PSMA-targeted C′ dot>EB-RT+C′ dot>EB-RT, C′ dot alone (ii) survival benefit (EB-RT+PSMA-targeted C′ dot+dual ICB)>EB-RT+non-targeted C′ dots+dual ICB>EB-RT, PSMA-targeted C′ dots, dual ICB alone.

Next, experiments can assess re-programming of the immunosuppressive prostate cancer TME in the Hi-Myc model using PSMA-targeted C′ dots. This includes evaluating time-dependent particle-induced alterations in pro-inflammatory markers using gene expression profiling.

Using a panel of 50 class-specific genes encompassing (i) iron metabolism; (ii) ferroptosis; (iii) antioxidant response; (iv) damage associated molecular patterns (DAMPS); (v) major histocompatibility class (MHC) class-I antigen processing/presentation; and (vi) immunomodulation, qRT-PCR can be used to investigate gene expression fold changes of tumor and splenic tissue specimens harvested 3-4 weeks post-treatment from representative long-term survivors (n=3 mice/cohort) of EB-RT+PSMA-targeted C dot-treated Hi-Myc mice, with and without dual ICB. Tissue samples can be bisected for both mRNA isolation and cytokine release assays. Luminex bead-based multiplex assays can be used to quantify cytokine secretion, as well as to validate differential mRNA levels of immunostimulatory and immunosuppressive cytokines noted during gene expression analysis.

Perform Immunophenotyping and Transcriptomics Characterization of Tumor-Infiltrating Leukocytes Derived from Histologic Specimens.

To confirm that changes in gene expression translate into phenotypic changes in the TME, CD45⁺ tumor-infiltrating leukocytes (TILs) from tumor and splenic tissues harvested 3-4 weeks post-treatment from representative long-term survivors (n=3 mice/cohort) can be isolated for immunophenotyping and scRNA/TCR-seq analysis. For immunophenotyping, multiparametric flow cytometry (MPFC) panel can be used, including those for NK-cells (CD11b⁺ NK1.1⁺), dendritic cells (DCs; CD11b⁺ CD11c⁺ MHCII⁺), M1 macrophages (CD11b⁺ MHCII⁺ Inos), M2 macrophages (CD11b⁺ MHCII⁺ CD206 Arginase1), cytotoxic T cells (CD3⁺ CD8⁺), Th1 cells (CD3⁺ CD4⁺ IFNγ Tbet⁺ CXCR3⁺), Th2 cells (CD3⁺ CD4⁺ GATA-3⁺ CCR4⁺), Tregs (CD3⁺ CD4⁺ CD25⁺ FoxP3⁺), monocytic myeloid derived suppressor cells (M-MDSC; CD11b⁺ Ly6G⁻ Ly6C^{high}) and granulocytic myeloid derived suppressor cells (G-MDSC; CD11b⁺ Ly6G⁺ Ly6C^{low}) can be used. Populational changes in these cell types for both particle-exposed and vehicle-treated cohorts (n=7 mice/cohort) can be analyzed and compared. In addition, indicators of improved (i) T cell effector function (↑CD8+/Treg), (ii) pro-inflammatory responses (↑Th1/Th2), and (iii) reduced immunosuppression (↑M1/M2, ↓percentage of MDSC within tumors) can be used to evaluate effective anti-tumor immune responses.

Evaluate Particle-Induced Alterations of Pro-Inflammatory Markers within the TME of the Hi-Myc Model Using Expression Profiling.

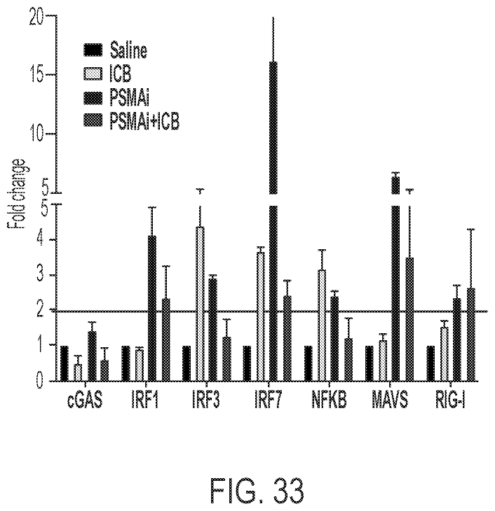

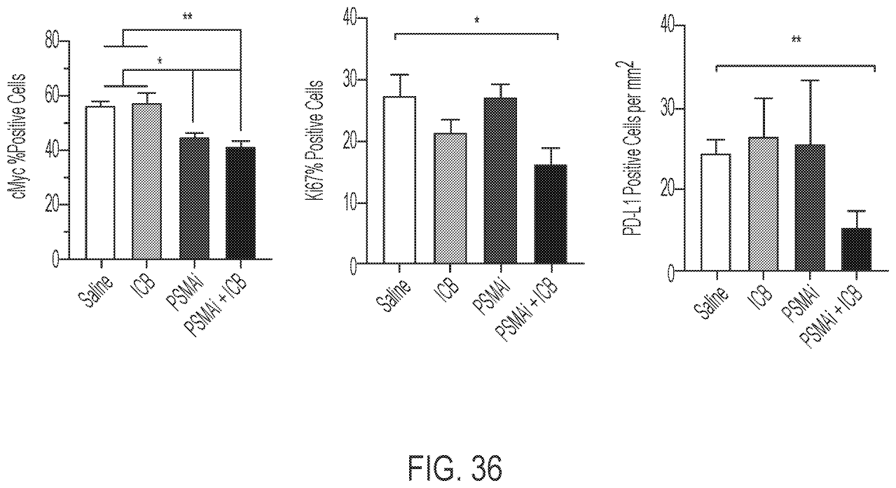

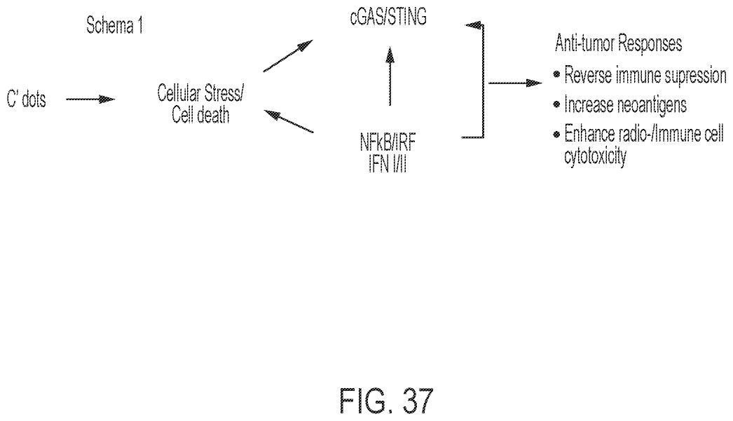

Preliminary gene expression profiling data was acquired in Hi-Myc mice stratified to 4 different treatment cohorts: (i) i.v.-injected non-radiolabeled PSMAi-PEG-Cy5-C′ dots (n=3 doses, 12 nmoles/dose), (ii) dual ICB (n=5 doses; e.g., anti-PD1/anti-CTLA4, i.p.-injected); (iii) non-radiolabeled PSMAi-PEG-Cy5-C′ dots followed by i.p.-injected dual ICB (n=5 doses), or (iv) saline vehicle (control). Mice were bred, genotyped, and allowed to mature to ˜24 weeks of age prior to studies. Animals were sacrificed 96 hours after the final treatment dose for histopathology and RT-qPCR analysis of tumor and splenic tissue specimens. Microscopy of H&E-stained tissue specimens showed multifocal prostatic intraepithelial neoplasia (PIN) lesions, a pre-neoplastic phenotype, within the ventral lobe, while the lateral lobe contained both multifocal PIN lesions and invasive carcinoma, the latter composed of multiple microinvasive lesions and a larger invasive carcinoma (FIG. 34). Gene expression profiles of prostate tumors harvested from these treatment cohorts are shown in FIG. 32B. Significant upregulation (e.g., fold changes greater than 2) of multiple expression signatures was found in particle- (as against vehicle-) treated tumor specimens (FIGS. 32B, 33, 36), including interferons (Ifna1, Ifnb1, Ifng), DAMPs (e.g., Hsp70a1), pro-inflammatory M1-macrophage markers (e.g., Nos2), and interleukins (e.g., IL12b). Further, interferon-stimulated gene (ISG) responses were also enhanced, including IRF7 (FIGS. 33, 36, upper panel) and Hsps, suggesting promotion of both innate and effector anti-tumor immunity. Surprisingly, RIG-1 and Mavs genes (FIG. 33) were also upregulated, part of a well-established pathway of innate immunity that drives production of IFNs and may synergize with STING pathway upregulation (FIG. 37). While treatment with ICB alone showed significant upregulation of a few genes (e.g., Ifna, Ifnb), combination dosing generally resulted in the highest transcription levels, notably for IFNs. Finally, statistically significant decreases in several tumor-specific, immune suppressive, and functional markers (e.g., cMyc, Ki-67, PD-L1) were seen by IHC (FIG. 36) relative to controls, most pronounced with combination therapy. No inhibitory modulation of the TME was seen at a systemic level (e.g., splenic tissue specimens, data not shown).

Using tissues from growth inhibition studies described herein, gene expression signatures in representative tumor and splenic tissue specimens harvested from all 4 specified cohorts of treated mice can be assessed; specimens can be bisected for mRNA isolation and cytokine release assays. A panel of 50 class-specific genes, informed by the described example, can include those related to (i) iron metabolism; (ii) ferroptosis; (iii) antioxidant responses; (iv) damage associated molecular patterns (DAMPS); (v) major histocompatibility class (MHC) class-I antigen processing/presentation; and (vi) immunomodulation. Data can be normalized for target and reference (e.g., Gapdh) genes, and results analyzed by the ΔΔCT method to obtain fold changes. To quantify cytokine secretion and validate differential mRNA levels of key stimulatory and suppressive cytokines identified by gene expression profiling, targeted cytokine proteome profiler assays can be used. Blood can be collected for assessing cytokine release profiles and toxicity. Tumor and major organs/tissues can be harvested for histopathology, including H&E, Ki-67, and IHC for DNA damage and cell death markers (STING, γH2AX, cleaved caspase-3).

### Example 3F: Perform Immunophenotyping and Transcriptomics Characterization of Tumor-Infiltrating Leukocytes Derived from Histologic Specimens and Examine Mechanisms that Reverse Immune Suppression

To confirm that changes in gene expression in translate into phenotypic changes in the TME, isolation of CD45+ tumor-infiltrating leukocytes (TILs) from tumor and splenic tissues harvested 3-4 weeks post-treatment from representative long-term survivors (n=3 mice/cohort) can be performed, as in for immunophenotyping and single-cell RNA and T cell receptor (TCR) sequencing (scRNA/TCR-seq). Preliminary data, acquired in tumor tissue specimens harvested from the Hi-Myc model 96 hours after four, high-dose non-targeted C′ dot injections, showed statistically significant decreases in several inhibitory T cell populations: PD-1 (FIG. 35, upper panel). Similarly, increases in MHC-II expressing myeloid cell populations (e.g., activated) were observed—all statistically significant (FIG. 35, lower panel). Importantly, upregulation of any immunosuppressive populations/markers in the spleen was not observed (data not shown). Together, these changes suggest that C′ dots have the potential to substantially reduce an immune suppressed TME without off-target effects prior to immunotherapy. In the studies described herein, analysis and comparison of populational changes in these cell types for both particle-exposed and vehicle-treated cohorts (n=7 mice/cohort) can be observed. For scRNA/TCR-seq experiments, two additional cohorts of mice (n=7/cohort) can be treated with lead particles or saline vehicle using the above procedures, and specimens (e.g., tumor, spleen) harvested and processed. CD45+(TILs)/CD45− (tumor) cells can be isolated from specimens, and T cell receptor (TCR)-repertoire analyses performed.

### In Vivo TME Biological Endpoints:

(i) increased T cell activity: CD8+/Treg; (ii) increased immunogenicity (increased TMB/neoantigen repertoire, increased MHC-I/II, increased Type I/II IFNs); (iii) reverse/limit suppression/resistance (decreased Tregs, decreased inhibitory T cell receptors/ligands (e.g., PD-1, LAG-3), increased antigen presentation).

### Example 3G: Perform Immunophenotypic and Transcriptomic Characterization of Tumor-Infiltrating Leukocytes Derived from Histologic Specimens and Examine Mechanisms that Reverse Immune Suppression

Preliminary data acquired from tumor tissue specimens harvested from the Hi-Myc model 96 hours after three PSMA-targeted C′ dot injections (12 nmoles/dose)+/−dual ICB, showed statistically significant increases in CD3+ and CD8+CXCR3+ TILs, as well as marked decreases in several inhibitory T cell populations, including PD-1+CD4 and CD8 T cell subsets and suppressive Tregs, most pronounced for combination therapy, leading to a significantly higher CD8+/Treg ratio (FIG. 34). In the myeloid panel (FIG. 35), statistically significant increases in MHC-II-expressing (e.g., activated) monocytes/macrophages and M1-like macrophages were also noted, with decreases in MDSCs and M2-like macrophages. Importantly, upregulation of immunosuppressive populations/markers in the spleen was not observed (data not shown). Together, these additive/synergistic changes suggest that this ultrasmall particle technology has the potential to substantially reduce immune suppression, improve overall survival, and increase responsiveness to ICB without adverse off-target effects.

### Key In Vivo TME Biological Endpoints.

To evaluate effective anti-tumor immune responses, lead PSMA-targeting C′ dots can be evaluated with and without ICB using the following endpoints: (i) increased cytotoxic T cell activity: CD8+/Treg; (ii) increased immunogenicity (increased TMB/neoantigen repertoire, increased MHC-I/II, increased Type I/II IFNs); (iii) reverse/limit suppression (decreased Tregs, decreased inhibitory T cell receptors and ligands (e.g., PD-1), increased antigen presentation).

### Pharmaceutically Acceptable Compositions

According to certain embodiments, the invention provides a composition comprising a nanoparticle as described herein and a pharmaceutically acceptable carrier, adjuvant, or vehicle. In certain embodiments, the amount of nanoparticle in administered compositions is such that is effective to measurably induce changes in immune cells of the tumor microenvironment, in a biological sample, or in a patient. In certain embodiments, a composition described herein is formulated for administration to a patient in need of such composition. In some embodiments, a composition is formulated for oral administration to a patient.

The term “patient,” as used herein, means an animal, preferably a mammal, and most preferably a human. In certain embodiments, the patient is a mouse.

The term “pharmaceutically acceptable carrier, adjuvant, or vehicle” refers to a non-toxic carrier, adjuvant, or vehicle that does not destroy the pharmacological activity of the nanoparticle with which it is formulated. Pharmaceutically acceptable carriers, adjuvants or vehicles that may be used in the compositions of various embodiments of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Compositions of certain embodiments of the present invention may be administered orally, parenterally, intraperitoneally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously. Sterile injectable forms of the compositions of certain embodiments of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Pharmaceutically acceptable compositions of certain embodiments described herein may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, pharmaceutically acceptable compositions of certain embodiments described herein may be administered in the form of suppositories for rectal administration. These can be prepared by mixing the agent with a suitable non-irritating excipient that is solid at room temperature but liquid at rectal temperature and therefore can melt in the rectum to release the drug. Such materials include cocoa butter, beeswax and polyethylene glycols.

Pharmaceutically acceptable compositions of certain embodiments described herein may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, provided pharmaceutically acceptable compositions may be formulated in a suitable ointment containing the active component suspended or dissolved in one or more carriers. Carriers for topical administration of compounds of embodiments described herein include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, provided pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions of certain embodiments of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Most preferably, pharmaceutically acceptable compositions of certain embodiments described herein are formulated for oral administration. Such formulations may be administered with or without food. In some embodiments, pharmaceutically acceptable compositions of certain embodiments described herein are administered without food. In other embodiments, pharmaceutically acceptable compositions of certain embodiments described herein are administered with food.

The amount of nanoparticles of certain embodiments described herein that may be combined with the carrier materials to produce a composition in a single dosage form can vary depending upon the host treated, the particular mode of administration. In certain embodiments, a dosage may be prepared to have a concentration of up to 100 μM of nanoparticles (e.g., up to 80 μM of nanoparticles). In certain embodiments, multiple dosage may be administered multiple times as part of a treatment regimen.

In certain embodiments, particles and one or more immunotherapies are administered sequentially. In other embodiments, particles and one or more immunotherapies are administered as a cocktail (e.g., for injection).

It should be understood that dose, dose frequency, timing of doses relative to one another and/or relative to other therapies may be evaluated and varied for a particular particle and/or a particular tumor type. Moreover, in certain embodiments, the number of doses of checkpoint inhibitors are varied, and/or the number and/or type of therapies used in combination are assessed for achieving maximal responses. In some embodiments, the described compositions comprising nanoparticles are administered before ICB. In some embodiments, the described compositions comprising nanoparticles are administered after ICB.

It should also be understood that a specific dosage and treatment regimen for any particular patient can depend upon a variety of factors, including the activity of the specific compound employed, the age, body weight, general health, sex, diet, time of administration, rate of excretion, drug combination, and the judgment of the treating physician and the severity of the particular disease being treated.

### Nanoparticle Compositions

In certain embodiments, the described compositions comprise ultrasmall nanoparticles or “C or C′ dots,” which are fluorescent, organo-silica core shell particles that have diameters controllable down to the sub-10 nm range with a range of modular functionalities. C or C′ dots are described by U.S. Pat. No. 8,298,677 B2 “Fluorescent silica-based nanoparticles”, U.S. Publication No. 2013/0039848 A1 “Fluorescent silica-based nanoparticles”, and U.S. Publication No. US 2014/0248210 A1 “Multimodal silica-based nanoparticles”, the contents of which are incorporated herein by reference in their entireties. Incorporated into the silica matrix of the core are near-infrared dye molecules, such as Cy5.5, which provides its distinct optical properties. Surrounding the core is a layer or shell of silica. The silica surface is covalently modified with silyl-polyethylene glycol (PEG) groups to enhance stability in aqueous and biologically relevant conditions. These particles have been evaluated in vivo and exhibit excellent clearance properties owing largely to their size and inert surface. Among the additional functionalities incorporated into C or C′ dots are chemical sensing, non-optical (PET) image contrast and in vitro/in vivo targeting capabilities, which enable their use in visualizing lymph nodes for surgical applications, and melanoma detection in cancer. C or C′ dots provide a unique platform for drug delivery due to their physical properties as well as demonstrated human in vivo characteristics. These compositions are ultrasmall (e.g., sub-8 nm) and benefit from targeted delivery (e.g., TLR-9) and EPR effects in tumor microenvironments, while retaining desired clearance and pharmacokinetic properties. To this end, in certain embodiments, drug constructs are covalently attached to C dots (or other nanoparticles). C dot-based compositions for drug delivery provide good biostability, minimize premature drug release, and exhibit controlled release of the bioactive compound. In certain embodiments, peptide-based linkers are used for the described compositions and other applications described herein. These linkers, in the context of antibodies and polymers, are stable both in vitro and in vivo, with highly predictable release kinetics that rely on enzyme catalyzed hydrolysis by lysosomal proteases. For example, cathepsin B, a highly expressed protease in lysosomes, can be utilized to facilitate drug release from macromolecules. By incorporating a short, protease sensitive peptide between the macromolecular backbone and the drug molecule, controlled release of the drug can be obtained in the presence of the enzyme. Interestingly, the described nanoparticles exhibit intrinsic therapeutic capabilities that (1) modulate the tumor microenvironment (TME) toward a pro-inflammatory phenotype, (2) increase immune cell activation and cytotoxicity in the TME, and (3) target cancer cells directly for cell death through the mechanism of ferroptosis.

In certain embodiments, the compositions comprises an ultrasmall (e.g., sub-50 nm diameter, e.g., sub-20 nm diameter, e.g., sub-15 nm diameter, e.g., sub-10 nm diameter, e.g., sub-8 nm diameter) silica nanoparticle containing a deep red/near-infrared dye (e.g., Cy5; absorption peak: 650 nm) that is covalently encapsulated within the silica-matrix. In this embodiment, due to the encapsulation of the dye and the specific design on the compositions, the brightness is dramatically improved (e.g., at least 2-times, e.g., at least 10-times, e.g., at least 50-times, e.g., at least 100-times, e.g., at least 600-times) as compared to the free dye.

In certain embodiments, cellular binding ligands (or targeting ligands) can be attached to the nanoparticle as described herein. In certain embodiments, the compositions comprise from 1 to 100 discrete targeting ligands (e.g., of the same type or different types), wherein these targeting ligands bind to receptors within/on tumor cells (e.g., wherein the compositions have an average diameter no greater than 15 nm, e.g., no greater than 10 nm, e.g., from about 5 nm to about 7 nm, e.g., about 6 nm). In certain embodiments, the compositions comprise a plurality of targeting ligands. In certain embodiments, the nanoparticles comprise from 1 to 100 discrete targeting ligands, e.g., from 1 to 30 discrete targeting ligands, e.g., from 1 to 20 discrete targeting ligands, e.g., from 1 to 10 discrete targeting ligands.

In certain embodiments, the nanoparticles comprise (e.g., has attached) one or more cellular binding ligands (or targeting ligands), e.g., for targeting cancer tissue/cells of interest. In certain embodiments, the composition comprises one or more cellular binding ligands (or targeting ligands) (e.g., attached thereto), such as, but not limited to, small molecules (e.g., folates, dyes, etc.), aptamers (e.g., A10, AS1411), polysaccharides, small biomolecules (e.g., folic acid, galactose, bisphosphonate, biotin), oligonucleotides, and/or proteins (e.g., (poly)peptides (e.g., αMSH, RGD, octreotide, AP peptide, epidermal growth factor, chlorotoxin, transferrin, etc.), antibodies, antibody fragments, proteins, etc.) or other peptides, antibody fragments, and/or immune modulatory components. In certain embodiments, the composition comprises one or more immune adjuvants (e.g., pattern recognition receptors, e.g., toll-like receptor agonists, e.g., antibody fragments) (and, optionally, a targeting agent). In certain embodiments, the composition comprises one or more contrast/imaging agents (e.g., fluorescent dyes, (chelated), MR-active agents, CT-agents), and/or therapeutic agents (e.g., small molecule drugs, therapeutic (poly)peptides, therapeutic antibodies, etc.).

The compositions may comprise one or more polymers (e.g., that may be associated with the nanoparticles), e.g., one or more polymers that have been approved for use in humans by the U.S. Food and Drug Administration (FDA) under 21 C.F.R. § 177.2600, including, but not limited to, polyesters (e.g., polylactic acid, poly(lactic-co-glycolic acid), polycaprolactone, polyvalerolactone, poly(1,3-dioxan-2-one)); polyanhydrides (e.g., poly(sebacic anhydride)); polyethers (e.g., polyethylene glycol); polyurethanes; polymethacrylates; polyacrylates; polycyanoacrylates; copolymers of PEG and poly(ethylene oxide) (PEO). In certain embodiments, the diameter of the compositions is not substantially increased by the one or more polymers.

The compositions may comprise one or more degradable polymers, for example, certain polyesters, polyanhydrides, polyorthoesters, polyphosphazenes, polyphosphoesters, certain polyhydroxyacids, polypropylfumerates, polycaprolactones, polyamides, poly(amino acids), polyacetals, polyethers, biodegradable polycyanoacrylates, biodegradable polyurethanes and polysaccharides. For example, specific biodegradable polymers that may be used include but are not limited to polylysine, poly(lactic acid) (PLA), poly(glycolic acid) (PGA), poly(caprolactone) (PCL), poly(lactide-co-glycolide) (PLG), poly(lactide-co-caprolactone) (PLC), and poly(glycolide-co-caprolactone) (PGC). Another exemplary degradable polymer is poly (beta-amino esters), which may be suitable for use in accordance with the present application.

In certain embodiments, a composition can have or be modified to have one or more functional groups. Such functional groups (within or on the surface of a nanoparticle) can be used for association with any agents (e.g., detectable entities, targeting entities, therapeutic entities, or PEG). In addition to changing the surface charge by introducing or modifying surface functionality, the introduction of different functional groups allows the conjugation of linkers (e.g., (cleavable or (bio-)degradable) polymers such as, but not limited to, polyethylene glycol, polypropylene glycol, PLGA, etc.), targeting/homing agents, and/or combinations thereof.

In certain embodiments, the composition comprises a therapeutic agent, e.g., a drug moiety (e.g., a chemotherapy drug). As used herein, “therapeutic agent” refers to any agent that has a therapeutic effect and/or elicits a desired biological and/or pharmacological effect, when administered to a subject.

In some embodiments, the compositions described herein demonstrate enhanced penetration of tumor tissue and diffusion within the tumor interstitium, e.g., for treatment of cancer, as described in International Patent Application No. PCT/US17/30056 (“Compositions and Methods for Targeted Particle Penetration, Distribution, and Response in Malignant Brain Tumors,” filed Apr. 28, 2016) by Bradbury et al., the contents of which is hereby incorporated by reference in its entirety. Further described are methods of targeting tumor-associated macrophages, microglia, and/or other cells in a tumor microenvironment using such nanoparticles conjugates.

In some embodiments, the compositions described herein can be used to induce ferroptosis, as described in International Patent Application No. PCT/US16/34351 (“Methods of Treatment Using Ultrasmall Nanoparticles to Induce Cell Death of Cancer Cells via Ferroptosis,” filed on May 26, 2016) by Bradbury et al., the contents of which is hereby incorporated by reference in its entirety. In some embodiments, the compositions described herein can be used to induce ferroptosis, as described in International Patent Application No. PCT/US18/63751 (“Methods of Cancer Treatment via Regulated Ferroptosis,” filed on Dec. 4, 2018) by Bradbury et al., the contents of which is hereby incorporated by reference in its entirety. In some embodiments, the compositions described herein can be used to activate innate and adaptive immune responses within the tumor microenvironment, as described in International Patent Application No. PCT/US19/66944 (“Inducing Favorable Effects on Tumor Microenvironment via Administration of Nanoparticle Compositions,” filed on Dec. 17, 2019, by Bradbury et al., the contents of which is hereby incorporated by reference in its entirety. In some embodiments, the compositions described herein can be used to activate tumor cells and/or innate and adaptive immune responses within the tumor microenvironment, as described in U.S. Provisional Application No. 63/212,930 (“Nanoparticle-mediated Enhancement of Immunotherapy to Promote Ferroptosis-induced Cytotoxicity and Antitumor Immune Responses,” filed on Jun. 21, 2021) by Bradbury et al., the contents of which is hereby incorporated by reference in its entirety.

Moreover, diagnostic, therapeutic, and theranostic (diagnostic and therapeutic) platforms featuring such compositions are described for treating targets in both the tumor and surrounding microenvironment, thereby enhancing efficacy of cancer treatment e.g., immunotherapies. Use of the compositions described herein with other conventional therapies, including chemotherapy, radiotherapy, immunotherapy, CAR T cell therapy, and the like, is also envisaged.

Moreover, use of fluorescent markers attached to (or incorporated in or on, or otherwise associated with) the nanoparticles provide quantitative assessment of compositions uptake at the target site and within the body, as well as permit monitoring of treatment response. In various embodiments, modular linkers are described for incorporating targeting ligands to develop a drug delivery system with controlled pharmacological properties. The described platforms determine the influence of targeting on compositions penetration and accumulation, thereby establishing an adaptable platform for improved delivery of a range of tractable SMIs, for example, to primary and metastatic brain tumors.

In certain embodiments, PET (Positron Emission Tomography) tracers are used as imaging agents. In certain embodiments, PET tracers comprise ⁸⁹Zr, ⁶⁴Cu, [¹⁸F] fluorodeoxyglucose.

In certain embodiments, the composition comprises one or more fluorophores, e.g., that may be associated with the nanoparticles. Fluorophores comprise fluorochromes, fluorochrome quencher molecules, any organic or inorganic dyes, metal chelates, or any fluorescent enzyme substrates, including protease activatable enzyme substrates. In certain embodiments, fluorophores comprise long chain carbophilic cyanines. In other embodiments, fluorophores comprise DiI, DiR, DiD, and the like. Fluorochromes comprise far red, and near infrared fluorochromes (NIRF). Fluorochromes include but are not limited to a carbocyanine and indocyanine fluorochromes. In certain embodiments, imaging agents comprise commercially available fluorochromes including, but not limited to methylene blue, Cy5.5, Cy5 and Cy7 (GE Healthcare); AlexaFlour660, AlexaFlour680, AlexaFluor750, and AlexaFluor790 (Invitrogen); VivoTag680, VivoTag-S680, and VivoTag-S750 (VisEn Medical); Dy677, Dy682, Dy752 and Dy780 (Dyomics); DyLight547, DyLight647 (Pierce); HiLyte Fluor 647, HiLyte Fluor 680, and HiLyte Fluor 750 (AnaSpec); IRDye 800CW, IRDye 800RS, and IRDye 700DX (Li-Cor); methylene blue; and ADS780WS, ADS830WS, and ADS832WS (American Dye Source) and Kodak X-SIGHT 650, Kodak X-SIGHT 691, Kodak X-SIGHT 751 (Carestream Health). In certain embodiments, a multi-wavelength camera as described by Bradbury et al. US Publication No. US 2015/0182118 A1, “Systems, Methods, and Apparatus for Multichannel Imaging of Fluorescent Sources in Real Time”, the disclosure of which is hereby incorporated by reference in its entirety. In certain embodiments, the imaging system used to image the lesion provides both static and functional assessments of the area of treatment (and its surroundings).

The surface chemistry, uniformity of coating (where there is a coating), surface charge, composition, concentration, frequency of administration, shape, and/or size of the compositions can be adjusted to produce a desired therapeutic effect.

In certain embodiments, the composition comprises a chelator (e.g., that may be associated with the nanoparticles), for example, 1,4,8,11-tetraazabicyclo[6.6.2]hexadecane-4,11-diyl)diacetic acid (CB-TE2A); desferoxamine (DFO); diethylenetriaminepentaacetic acid (DTPA); 1,4,7, 10-tetraazacyclotetradecane-1,4,7, 10-tetraacetic acid (DOTA); thylenediaminetetraacetic acid (EDTA); ethylene glycolbis(2-aminoethyl)-N,N,N′,N′-tetraacetic acid (EGTA); 1,4,8,11-tetraazacyclotetradecane-1,4,8,11-tetraacetic acid (TETA); ethylenebis-(2-4 hydroxy-phenylglycine) (EHPG); 5-Cl-EHPG; 5Br-EHPG; 5-Me-EHPG; 5t-Bu-EHPG; 5-sec-Bu-EHPG; benzodiethylenetriamine pentaacetic acid (benzo-DTPA); dibenzo-DTPA; phenyl-DTPA, diphenyl-DTPA; benzyl-DTPA; dibenzyl DTPA; bis-2 (hydroxybenzyl)-ethylene-diaminediacetic acid (HBED) and derivatives thereof, Ac-DOTA; benzo-DOTA; dibenzo-DOTA; 1,4,7-triazacyclononane N,N′,N″-triacetic acid (NOTA); benzo-NOTA; benzo-TETA, benzo-DOTMA, where DOTMA is 1,4,7, 10-tetraazacyclotetradecane-1,4,7,10-tetra(methyl tetraacetic acid), benzo-TETMA, where TETMA is 1,4,8,11-tetraazacyclotetradecane-1,4,8,11-(methyl tetraacetic acid); derivatives of 1,3-propylenediaminetetraacetic acid (PDTA); triethylenetetraaminehexaacetic acid (TTHA); derivatives of 1,5,10-N,N′,N″-tris(2,3-dihydroxybenzoyl)-tricatecholate (LICAM); and 1,3,5-N,N′,N″-tris(2,3-dihydroxybenzoyl)aminomethylbenzene (MECAM), or other metal chelators.

In certain embodiments, the composition comprises an azide moiety, e.g., that may be associated with the nanoparticles. In certain embodiments, an azide moiety is attached to an antibody fragment for conjugation to a nanoparticle described herein. In certain embodiments, azide moieties are attached to ligands, for instance, for conjugation to a nanoparticle described herein.

What is claimed:

1. A method of treatment of a subject having been diagnosed with cancer (e.g. prostate cancer, ovarian cancer, a malignant brain tumor (e.g., brain or spinal), melanoma), the method comprising administering (e.g., via IV administration) to the subject a composition comprising ultrasmall nanoparticles in concert with administering one or more of (i) to (iv) as follows:
(i) cellular therapy;
(ii) one or more immune checkpoint blockade antibodies (ICB);
(iii) one or more pharmacologic inhibitors; and
(iv) external beam radiation or molecular radiotherapy (e.g., peptide radioligands (e.g., radiolabeled PSMA-targeting ligands)).

2. The method of claim 1, comprising administering external beam radiation or radiotherapy, wherein the nanoparticles in combination with radiation enhance efficacy of checkpoint blockade.

3. The method of claim 1 or 2, comprising administering radiotherapy, wherein the molecular radiotherapy comprises a radiotherapeutic label.

4. The method of claim 3, wherein the radiotherapeutic label comprises an alpha-emitting radioisotope or a beta-emitting radioisotope.

5. The method of claim 4, wherein the alpha-emitting radioisotope comprises ²²⁵Ac.

6. The method of claim 4, wherein the beta-emitting radioisotope comprises ¹⁷⁷Lu.

7. The method of any one of claims 1 to 6, comprising administering cellular therapy, wherein the cell therapy comprises T-cell therapy or engineered cell therapy (e.g., CAR T cell therapy).

8. The method of any one of claims 1 to 7, comprising administering one or more pharmacologic inhibitors, wherein the pharmacologic inhibitors comprise one or more myeloid cell-targeting inhibitors.

9. A method of treatment of a subject having been diagnosed with cancer (e.g., ovarian cancer), the method comprising:
(i) administering (e.g., via IV administration) to the subject a composition comprising ultrasmall nanoparticles; and
(ii) administering to the subject cells.

10. The method of claim 1, wherein the cells comprise engineered cells.

11. The method of claim 9 or 10, wherein the cells comprise engineered cells, wherein the engineered cells comprise CAR T cells, and wherein the nanoparticles (i) augment intratumoral immune responsiveness and cytotoxicity and/or (ii) improves CAR T cell exhaustion or enhances CAR T cell persistence.

12. The method of any one of claims 9 to 11, wherein the nanoparticles comprise tumor-targeting ligands.

13. The method of any one of claims 9 to 12, wherein the nanoparticles do not comprise tumor-targeting ligands.

14. A method of treatment of a subject having been diagnosed with cancer (e.g., tumors comprised of high levels of myeloid cells, a main driver of immune evasion e.g., melanoma or triple negative breast cancer, TNBC), the method comprising:
(i) administering (e.g., via IV administration) to the subject a composition comprising ultrasmall nanoparticles, said nanoparticles comprising targeting ligands;
(ii) administering to the subject a myeloid-targeting inhibitor; and
(iii) administering to the subject one or more immune checkpoint blockade antibodies.

15. The method of claim 14, wherein the cancer comprises one or more tumors comprised of high levels of myeloid cells.

16. The method of claim 14 or 15, wherein the targeting ligands comprise melanocortin-1 receptor (MC1-R) targeting ligands of a single type or multiple types.

17. The method of claim 16, wherein the myeloid-targeting inhibitor comprises a PI3Kγ-selective inhibitor targeting myeloid cells (e.g., IPI-549).

18. The method of any one of claims 14 to 17, wherein the one or more immune checkpoint blockade antibodies comprises a member selected from the group consisting of ipilimumab (anti-CTLA4), pembrolizumab (anti-PD1), nivolumab (anti-PD1), and atezolizumab (anti-PD-L1).

19. The method of any one of claims 14 to 18, wherein resistance to immune ICB is limited by combining particle-driven cytotoxic responses and enhanced pro-inflammatory responses with one or more immune checkpoint blockade antibodies and/or selective PI3Kγ-targeting to subvert immunosuppressive components in a tumor microenvironment (TME).

20. A method of treatment of a subject having been diagnosed with cancer (e.g., prostate cancer), the method comprising:
(i) administering (e.g., via IV administration) to the subject a composition comprising ultrasmall nanoparticles, said nanoparticles comprising targeting ligands;
(ii) administering to the subject external beam radiotherapy or molecular radiotherapy (e.g., peptide radioligands (e.g., radiolabeled PSMA-targeting ligands)); and
(iii) administering to the subject one or more immune checkpoint blockade antibodies.

21. The method of claim 20, wherein the targeting ligands comprise PSMA-targeting ligands of a single type or multiple types.

22. The method of claim 20 or 21, wherein the molecular radiotherapy comprises a radiotherapeutic label.

23. The method of claim 22, wherein the radiotherapeutic label comprises an alpha-emitting radioisotope or a beta-emitting radioisotope.

24. The method of claim 23, wherein the alpha-emitting radioisotope comprises ²²Ac.

25. The method of claim 23, wherein the beta-emitting radioisotope comprises ¹⁷Lu.

26. The method of any one of claims 20 to 25, wherein the one or more immune checkpoint blockade antibodies comprises a member selected from the group consisting of ipilimumab (anti-CTLA4), pembrolizumab (anti-PD1), nivolumab (anti-PD1), and atezolizumab (anti-PD-L1).

27. The method of any one of claim 20 to 26, further comprising administering to the subject a myeloid-targeting inhibitor.

28. The method of claim 27, wherein the myeloid-targeting inhibitor comprises a PI3Kγ-selective inhibitor targeting myeloid cells (e.g., IPI-549).

29. The method of any one of the preceding claims, wherein the nanoparticles have a diameter no greater than 20 nm.

30. The method of any one of the preceding claims, wherein the nanoparticles have a diameter no greater than 10 nm.

31. The method of any one of the preceding claims, wherein the nanoparticles comprise silica.

32. The method of any one of the preceding claims, wherein the cancer comprises prostate cancer, ovarian cancer, malignant brain tumors, melanoma, breast cancer, or lung cancer.

33. The method of any one of the preceding claims, wherein each of the nanoparticles comprises 1 to 25 targeting ligands.

34. The method of claim 33, wherein the targeting ligand is a targeting ligand for a cellular receptor.

35. The method of claim 34, wherein the targeting ligand for a cellular receptor comprises MC1-R or PSMA.

36. The method of any one of the preceding claims, wherein each of the nanoparticles has a hydrodynamic diameter no greater than 20 nm.

37. The method of any one of the preceding claims, wherein each of the nanoparticles has a hydrodynamic diameter no greater than 10 nm.

38. The method of any one of the preceding claims, wherein each of the nanoparticles comprises a silica core.

39. The method of claim 38, wherein the silica core has a diameter less than 10 nm.

40. The method of any one of the preceding claims, wherein each of the nanoparticles comprises a polyethylene glycol (PEG) shell.

41. The method of claim 40, wherein the thickness of the PEG shell is less than 2 nm.

42. The method of any one of the preceding claims, wherein the nanoparticle comprises a chelator.

43. The method of claim 42, wherein the chelator is selected from the group comprising 1,4,7,10-tetraazacyclododecane-1,4,7,10-tetraacetic acid (DOTA), DOTA-Bz-SCN, and desferoxamine (DFO).

44. The method of any one of the preceding claims, wherein local concentration of nanoparticles within a microenvironment of a tumor of the subject is in a range from about 0.013 nmol/cm³ to about 86 nmol/cm³ or from about 0.013 nmol/cm³ to about 0.14 nmol/cm3 or from about 8 nmol/cm³ to about 86 nmol/cm³.

45. The method of claim 55, wherein an administered dose (e.g., by IV administration) has a particle concentration from about 100 nM to about 60 μM, or wherein an administered dose has particle concentration less than 150 nM.

46. The method of claim 44, wherein an administered dose has particle concentration greater than or equal to about 1 μM.

47. A composition for use in the method of any one of the preceding claims, the composition comprising ultrasmall nanoparticles having the following attributes:
(i) a number of targeting ligands from 5 to 60 per nanoparticle;
(ii) a heterogeneous surface characterized by one or more of (a) to (d) as follows:
(a) an unincorporated dye;
(b) a variation in a polyethylene glycol (PEG) coating;
(c) a variation in dye encapsulation; and
(d) a variation in number of targeting ligands;
(iii) a particle core and shell having a hydrodynamic diameter in a range from 4.7 nm to 7.8 nm; and
(iv) a silica composition controlled for ferroptosis.

48. The composition of claim 47, wherein the silica composition controlled for ferroptosis comprises nanoparticles made using a ratio of phosphonate-silane to tetramethyl orthosilicate (TMOS) in a reaction feed at or above 20% such that ferroptosis may occur.

49. The composition of claim 47, wherein the silica composition controlled for ferroptosis comprises nanoparticles made using a ratio of phosphonate-silane to tetramethyl orthosilicate (TMOS) in a reaction feed from 0% to 20% such that ferroptosis may not occur.

50. The composition of any one of claims 47 to 49, wherein the ultrasmall nanoparticles have a heterogenous surface characterized by a variation in a polyethylene glycol (PEG) coating, wherein the PEG coating comprises from about 100 to about 500 PEG chains per nanoparticle.

51. The composition of any one of claims 47 to 50, wherein the ultrasmall nanoparticles have a heterogenous surface characterized by wherein the dye encapsulation is by PEG.

52. The composition of any one of claims 47 to 51, wherein the ultrasmall nanoparticles have a heterogenous surface characterized by a variation in number of targeting ligands, wherein the targeting ligands range from 1 to 60 per nanoparticle, or from 1 to 15 per nanoparticle, or from 40 to 60 per nanoparticle.

53. A method of identifying a treatment for a subject or group of subjects involving administration of silica nanoparticles, said method comprising identifying one or more biomarkers.

54. The method of claim 53, wherein the one or more biomarkers comprise one or more of the following:
Markers for Immunophenotyping

| Marker | Cell Type |
|---|---|
| Th1 cells | CD3+, CD4+, IFN-γ+, Tbet+ CXCR3+ |
| Th2 cells | CD3+, CD4+, GATA-3+, CCR4+ |
| Activated CD8+ T | CD3+, CD8+, CXCR3+ |
| cells | |
| Tregs | CD3+, CD4+. CD25+, FoxP3+ |
| T helpers | CD3+, CD4+, FoxP3− |
| Inhibitory T cells | CD8/CD4 (LAG3+, TIM-3+, PD1+, CTLA4+) |
| M-MDSC | CD11b+, Ly6G−, LY6Chigh |
| G-MDSC | CD11b+, Ly6G+, LY6Clow |
| Macrophage M1 | CD11b+, MHCII+, iNOS |
| Macrophage M2 | CD11b+, MCHII+, CD206+, Arginase1+ |
| Dendritic cells | CD11b+, CD11c+, MHCII+ (CD40, CD80, CD86) |
| (DCs) | |
| Monocytes | CD11b+, LY6G+, MGCII+ |
| Neutrophils | Ly6G+/− |
| Natural Killer (NK) | NK1.1 |
| cells | |

55. The method of claim 53 or 54, wherein the one or more biomarkers comprise one or more of the following:
Markers for Adaptive Immune Response

| Gene | Gene | | |
|---|---|---|---|
| Number | Symbol | Gene Name | Annotation |
| 1 | Ccl20 | Chemokine (C-C motif) | Binds to CCR6. Recruits DCs, effector/memory |
| | | ligand 20 | T-cells and B-cells, Th17 and Treg cells. |
| 2 | Ccl22 | Chemokine (C-C motif) | Binds to CCR4. In OC, TAMs secrete CCL22 to |
| | | ligand 22 | attract Treg cells. (PMID: 28555670). |
| 3 | Cd401g | CD40 ligand | Binds to CD40. Upon binding to CD40 (CD138) |
| | | | on DCs, CD40L mediates CD8+ T-cell immunity |
| | | | via secretion of IL-12 and IFN-γ. Some CAR-T |
| | | | cells are designed to express CD40L (PMID: |
| | | | 27068948). |
| 4 | Csf1 | Colony stimulating | Plays an essential role in the regulation of |
| | | factor 1 (macrophage) | survival, proliferation and differentiation of |
| | | | macrophages and monocytes. Blocking Csf1/Csf1R |
| | | | signaling in the TME decreases TAM (PMID: 25082815). |
| 5 | Cxcl10 | Chemokine (C-X-C | Ligand for CXCR3. Increased CXCL10 is |
| | | motif) ligand 10 | associated with increased CD8 T infiltrating |
| | | | tumor cells. Elevated serum levels of CXCL9 and |
| | | | 10 are associated with higher survival rates in |
| | | | patients with OC (PMID: 27490802). |
| 6 | Cxcl12 | Chemokine (C-X-C | Ligand for CXCR4. Recruits Treg cells. (PMID: |
| | | motif) ligand 12 | 26629936) |
| 7 | Cxcl5 | Chemokine (C-X-C | Ligand for CXCR2. Involved in neutrophil |
| | | motif) ligand 5 | activation. Associated with late stage gastric |
| | | | cancer (PMID: 17479287) and poor survival in |
| | | | pancreatic cancer (PMID: 21356384). Recruits |
| | | | MDSC (PMID: 28555670). |
| 8 | Cxcl9 | Chemokine (C-X-C | Ligand for CXCR3. CD8 T cells, Th1 and NK |
| | | motif) ligand 9 | cells express CXCR3. Increased CXCL10 is |
| | | | associated with increased CD8 T infiltrating |
| | | | tumor cells. Elevated serum levels of CXCL9 and |
| | | | 10 are associated with higher survival rates in |
| | | | patients with ovarian cancer (PMID: 27490802). |
| 9 | Fasl | Fas ligand (TNF | Involved in cytotoxic T-cell-mediated apoptosis, |
| | | superfamily; | natural killer cell-mediated apoptosis and in T- |
| | | member 6) | cell development (PMID: 9228058, PMID: |
| | | | 7528780, PMID: 9427603). High levels of FasL |
| | | | in the tumor stroma decrease CD8 T cell |
| | | | infiltration in tumors (PMID: 24793239) and help |
| | | | tumor escape immune attack (PMID: 17667919). |
| 10 | Ifng | Interferon gamma | Secreted by Th1 and Type I NKT cells. Activates |
| | | | CD8 T cells and DCs. |
| 11 | Ifna1 | Interferon alpha-1 | Produced by macrophages, IFN-alpha have antiviral |
| | | | activities. |
| 12 | Il10 | Interleukin 10 | Ligand for the heterotetrameric receptor |
| | | | comprised of IL10RA and IL10RB. Targets |
| | | | macrophages and monocytes and limits their |
| | | | release of pro-inflammatory cytokines including |
| | | | GM-CSF, IL-1 alpha, IL-1 beta, IL-6, IL-8 and |
| | | | TNF-alpha (PMID: 1940799, PMID: 7512027, |
| | | | PMID: 11564774). Interferes with antigen |
| | | | presentation by reducing the expression of MHC- |
| | | | class II and co-stimulatory molecules, thereby |
| | | | inhibiting their ability to induce T cell activation |
| | | | (PMID: 8144879). |
| 13 | Il12b | Interleukin 12B | Acts as a growth factor for activated T and NK |
| | | | cells, enhance the lytic activity of NK/ |
| | | | lymphokine-activated killer cells, and stimulate |
| | | | the production of IFN-gamma. |
| 14 | Il13 | Interleukin 13 | Ligand for IL-13Rα1 and IL-13Rα2. Interacts |
| | | | with IL4 to mediate tumor progression but |
| | | | synergizes with IL2 to increase production of |
| | | | IFN-γ (PMID: 8096327). |
| 15 | Il15 | Interleukin 15 | Activates T cells and NK cells. IL15 agonist |
| | | | ALT803 enhances NK function against ovarian |
| | | | cancer (PMID: 30410679, 28236454). |
| 16 | Il17a | Interleukin 17A | Ligand for IL17RA and IL17RC. Induces stromal |
| | | | cells to produce proinflammatory and |
| | | | hematopoietic cytokines (PMID: 8676080). |
| | | | Stimulates CD133+ cells in OC and drives tumor |
| | | | progression (PMID: 24362529). |
| 17 | Il1a | Interleukin 1 alpha | IL1A is a potential diagnostic biomarker for |
| | | | NSCLC (PMID: 25554695). |
| 18 | Il1b | Interleukin 1 beta | Promotes Th17 differentiation of T-cells. |
| 19 | Il2 | Interleukin 2 | Produced by T-cells in response to antigenic or |
| | | | mitogenic stimulation, it is required for T-cell |
| | | | proliferation. |
| 20 | Il23a | Interleukin 23, alpha | Promotes inflammatory responses and increases |
| | | subunit p19 | angiogenesis. Necessary for activation of Th17 |
| | | | cells. |
| 21 | Il4 | Interleukin 4 | Immunosuppressive cytokine in the TME |
| | | | (PMID: 28733709). Activates Th2 cells. |
| 22 | Il6 | Interleukin 6 | Increased levels of IL6 are associated with poor |
| | | | prognosis in patients (PMID: 21795409). |
| | | | Involved in lymphocyte and monocyte |
| | | | differentiation. Required for the generation of |
| | | | Th17 and Treg cells. |
| 23 | Il7 | Interleukin 7 | Decreased Treg tumor infiltration and apoptosis |
| | | | of T cells (PMID: 19454692). |
| 24 | Il9 | Interleukin 9 | Supports IL-2 and IL-4 independent growth of |
| | | | helper T-cells. (PMID: 27832300). |
| 25 | Inha | Inhibin alpha | Activates T cells. |
| 26 | Nfkb1 | Nuclear factor of kappa | Elevated expression associated with poor |
| | | light polypeptide gene | survival in newly diagnosed patients in OC |
| | | enhancer in B-cells 1, | (PMID: 20564628 - 2010). |
| | | p105 | |
| 27 | Nos2 | Nitric oxide synthase 2, | M1 macrophage marker. |
| | | inducible | |
| 28 | Tnf | Tumor necrosis factor- | In the TME increases myeloid cell recruitment in |
| | | alpha | an IL-17-dependent manner that contributes to |
| | | | tumor growth (PMID: 19741298). |
| 29 | Tgfb1 | TGF-Beta 1 | Can promote Th17 or Treg lineage differentiation |
| | | | by expression of Foxp3 (PMID: 14676299 - 2003). |
| 30 | Prf1 | Perforin 1 | Cytolytic protein produced by T and NK cells. |
| 31 | Gzma | Granzyme A | Cytolytic protein produced by T and NK cells. |

Markers for Innate Immune Response, Antigen Presentation and DAMPs

| Gene | Gene | | |
|---|---|---|---|
| Number | Symbol | Gene Name | Annotation |
| 32 | H2-D1 | MHC Ia, Histocompatibility | Involved in the presentation of foreign |
| | | 2, D1 | antigens to the immune system. |
| 33 | H2-T23 | MHC Ib, H-2 class I | Involved in the presentation of foreign |
| | | histocompatibility antigen, | antigens to the immune system. |
| | | D-37 alpha chain | |
| 34 | B2M | Beta-2-microglobulin | Component of the class I major |
| | | | histocompatibility complex (MHC). |
| | | | Involved in Ag presentation to the immune |
| | | | system. |
| 33 | TAP1 | Antigen peptide transporter 1 | Involved in the transport of antigens from |
| | | | the cytoplasm to the endoplasmic reticulum |
| | | | for association with MHC class I molecules. |
| 34 | TAP2 | Antigen peptide transporter 2 | Involved in the transport of antigens from |
| | | | the cytoplasm to the endoplasmic reticulum |
| | | | for association with MHC class I molecules. |
| 35 | Calr | Calreticulin | Translocation to the extracellular space is |
| | | | an early indicator of cell stress response |
| | | | (PMID: 18573340). |
| 36 | Hspa1b | Heat shock 70 kDa protein 1B | Early indicator of cell stress response |
| | | | (PMID: 18573340). |
| 37 | Hsp90ab1 | Heat shock protein HSP 90- | Early indicator of cell stress response |
| | | beta | (PMID: 18573340). |
| 38 | IFNB1 | Interferon Beta I | Indicator of innate immune response. |
| 39 | IFNA1 | Interferon Alpha I | Indicator of innate immune response. |
| 40 | IFI204 | Interferon-activable protein | Essential for IRF3 and NF-kB activation |
| | | 204 | and induction of IFN beta 1. |
| 41 | IFI44 | Interferon-induced protein 44 | Upregulated in OC (PMID: 17145569). |
| 42 | HMGB1 | High mobility group box 1 | DAMPs marker |
| | | protein | |
| 43 | S100A8 | S100 calcium-binding protein | DAMPs marker |
| | | A8 | |
| 44 | S100A9 | S100 calcium-binding protein | DAMPs marker |
| | | A9 | |
| 45 | STING | Stimulator of interferon genes | DAMPs marker |

Markers for Ferroptosis, Iron metabolism and Antioxidant

| Gene | Gene | | |
|---|---|---|---|
| Number | Symbol | Gene Name | Annotation |
| 46 | Fth1 | Ferritin Heavy Chain | Stores iron in a soluble, non-toxic, readily |
| | | | available form. |
| 47 | Aco1 | Aconitase 1 | When cellular iron levels are high, the |
| | | | encoded protein functions as an aconitase, |
| | | | an essential enzyme in the TCA cycle that |
| | | | catalyzes the conversion of citrate to |
| | | | isocitrate. When cellular iron levels are low, |
| | | | the encoded protein regulates iron uptake |
| | | | and utilization by binding to iron-responsive |
| | | | elements in the untranslated regions of |
| | | | mRNAs for genes involved in iron metabolism. |
| 48 | Tfrc | Transferrin receptor | Iron uptake via receptor-mediated endocytosis. |
| 49 | Slc40a1 | Ferroportin-1 | Iron exporter. |
| 50 | Alas2 | Aminolevulinic acid synthase | Locates in the mitochondria. Maintains iron |
| | | 2, erythroid | homeostasis. |
| 51 | Slc3a2 | 4F2 cell-surface antigen heavy | (IFNγ) released from CD8+ T cells |
| | | chain | downregulates the expression of SLC3A2 |
| | | | and SLC7A11, two subunits of the |
| | | | glutamate-cystine antiporter system xc−, |
| | | | impairs the uptake of cystine by tumour |
| | | | cells, and as a consequence, promotes |
| | | | tumour cell lipid peroxidation and |
| | | | ferroptosis. (PMID: 31043744) |
| 52 | Slc7a11 | Cystine/glutamate transporter | (IFNγ) released from CD8+ T cells |
| | | | downregulates the expression of SLC3A2 |
| | | | and SLC7A11, two subunits of the |
| | | | glutamate-cystine antiporter system xc−, |
| | | | impairs the uptake of cystine by tumour |
| | | | cells, and as a consequence, promotes |
| | | | tumour cell lipid peroxidation and |
| | | | ferroptosis. (PMID: 31043744) |
| 53 | Acs14 | Long-chain-fatty-acid--CoA | Cells resistant to ferroptosis exhibit reduced |
| | | ligase 4 | levels of Acsl4. |
| 54 | Gpx4 | Glutathione peroxidase 4 | Protects cells against membrane lipid |
| | | | peroxidation. |
| 55 | Nfe2l2 | NRF2 | Transcription factor controlling antioxidant |
| | | | genes including GCLC and NQO1. |
| 56 | Nqo1 | NAD(P)H quinone | Antioxidant enzyme regulated by NRF2. |
| | | dehydrogenase 1 | |
| 57 | Sod2 | Superoxide dismutase 2, | Transforms toxic superoxide into hydrogen |
| | | mitochondrial | peroxide. |
| 58 | Gclc | Glutamate-cysteine ligase | First enzyme in the glutathione (GSH) |
| | | catalytic subunit | biosynthesis pathway. |
| 59 | Gss | Glutathione synthetase | Second enzyme in the glutathione (GSH) |
| | | | biosynthesis pathway. |

56. The method of claim 55, wherein the method is performed via real-time quantitative reverse transcription polymerase chain reaction (qRT-PCR).

57. The method of claim 53 or 56, wherein the method comprises identifying one or more pattern recognition receptors (e.g., STING (Stimulator of interferon genes), TLR (Toll-like receptor), RIG-I (Retinoic acid-inducible gene I) biomarkers.

58. A kit comprising a nanoparticle composition in a unit dosage effective for enhanced treatment of cancer in a subject having been diagnosed with the cancer and receiving (or having received) therapy with engineered cells, the nanoparticle composition comprising ultrasmall nanoparticles.

59. The kit of claim 58, wherein the nanoparticles comprise tumor-targeting ligands.

60. The kit of claim 58, wherein the nanoparticles do not comprise tumor-targeting ligands.

61. The kit of any one of claims 58 to 60, wherein the composition of the nanoparticle (i) augments intratumoral immune responsiveness and cytotoxicity and/or (ii) improves CAR T cell exhaustion and/or persistence.

62. A kit comprising a nanoparticle composition in a unit dosage effective for enhanced treatment of cancer (e.g., one or more tumors having a high level of myeloid cells, a main driver of immune evasion e.g., melanoma or triple negative breast cancer, TNBC) in a subject having been diagnosed with the cancer and receiving (or having received) therapy with a myeloid-targeting inhibitor and one or more immune checkpoint blockade antibodies, the nanoparticle composition comprising ultrasmall nanoparticles, said nanoparticles comprising targeting ligands.

63. The kit of claim 62, wherein the cancer comprises one or more tumors comprised of high levels of myeloid cells.

64. The kit of claim 62 or 63, wherein the myeloid-targeting inhibitor comprises a PI3Kγ-selective inhibitor targeting myeloid cells (e.g., IPI-549).

65. The kit of any one of claims 62 to 64, wherein the one or more immune checkpoint blockade antibodies comprises a member selected from the group consisting of ipilimumab (anti-CTLA4), pembrolizumab (anti-PD1), nivolumab (anti-PD1), and atezolizumab (anti-PD-L1).

66. The kit of any one of claims 62 to 65, wherein the targeting ligands comprise melanocortin-1 receptor (MC1-R) targeting ligands of a single type or multiple type.

67. The kit of any one of claims 62 to 66, wherein resistance to immune ICB is limited by combining particle-driven cytotoxic processes (e.g., ferroptosis, immune-related cell death) and enhanced pro-inflammatory responses with one or more immune checkpoint blockade antibodies and/or selective PI3Kγ-targeting to subvert immunosuppressive components in a tumor microenvironment (TME).

68. A kit comprising (a) a nanoparticle composition in a unit dosage effective for enhanced treatment of cancer (e.g., prostate cancer) in a subject having been diagnosed with the cancer and receiving (or having received) therapy with one or more immune checkpoint blockade antibodies, the nanoparticle composition comprising ultrasmall nanoparticles, said nanoparticles comprising targeting ligands; and (b) a molecular radiotherapeutic, e.g., a composition comprising peptide radioligands (e.g., radiolabeled PSMA-targeting ligands) (e.g., further comprising a myeloid-targeting inhibitor (e.g., a PI3Kγ-selective inhibitor targeting myeloid cells (e.g., IPI-549))).

69. The kit of claim 68, wherein the molecular radiotherapeutic comprises a radiotherapeutic label.

70. The kit of claim 69, wherein the radiotherapeutic label comprises an alpha-emitting radioisotope or a beta-emitting radioisotope.

71. The kit of claim 70, wherein the alpha-emitting radioisotope comprises ²²⁵Ac.

72. The kit of claim 70, wherein the beta-emitting radioisotope comprises ¹⁷⁷Lu.

73. The kit of any one of claims 68 to 72, further comprising the one or more immune checkpoint blockade antibodies, wherein the one or more immune checkpoint blockade antibodies comprises one or more members selected from the group consisting of ipilimumab (anti-CTLA4), pembrolizumab (anti-PD1), nivolumab (anti-PD1), and atezolizumab (anti-PD-L1).

74. The kit of any one of claims 68 to 73, wherein the targeting ligands comprise PSMA-targeting ligands.

75. The kit of any of claims 68 to 74, wherein the nanoparticles have a diameter no greater than 20 nm.

76. The kit of any of claims 58 to 75, wherein the nanoparticles have a diameter no greater than 10 nm.

77. The kit of any of claims 58 to 76, wherein the nanoparticles comprise silica.

78. The kit of any one of claims 58 to 76, wherein each of the nanoparticles comprises 1 to 25 targeting ligands.

79. The kit of any one of claims 58 to 78, wherein the cancer comprises prostate cancer, ovarian cancer (e.g., high-grade ovarian cancer), malignant brain tumors, melanoma, breast cancer, or lung cancer.

80. A method comprising administering to a subject a nanoparticle composition in a unit dosage effective for enhanced treatment of cancer (e.g., ovarian cancer), wherein the subject has been diagnosed with the cancer and is receiving (or has received) therapy with cells (e.g., T cells, e.g., dendritic cells, e.g., engineered cells, e.g., chimeric antigen receptor (CAR) T-cells), the nanoparticle composition comprising ultrasmall nanoparticles.

81. The method of claim 80, wherein the nanoparticles comprise tumor-targeting ligands.

82. The method of claim 80, wherein the nanoparticles do not comprise tumor-targeting ligands.

83. The method of any one of claims 80 to 82, wherein the nanoparticle composition (i) augments intratumoral immune responsiveness and cytotoxicity and/or (ii) improves CAR T cell exhaustion.

84. A method comprising administering to a subject a nanoparticle composition in a unit dosage effective for enhanced treatment of cancer (e.g., one or more tumors having a high level of myeloid cells, a main driver of immune evasion e.g., melanoma or triple negative breast cancer, TNBC), wherein the subject has been diagnosed with the cancer and is receiving (or has received) therapy with a myeloid-targeting inhibitor and one or more immune checkpoint blockade antibodies, the nanoparticle composition comprising ultrasmall nanoparticles, said nanoparticles comprising targeting ligands.

85. The method of claim 84, wherein the cancer comprises one or more tumors having a high level of myeloid cells.

86. The method of claim 84 or 85, wherein the myeloid-targeting inhibitor comprises a PI3Kγ-selective inhibitor targeting myeloid cells (e.g., IPI-549).

87. The method of any one of claims 84 to 86, wherein the one or more immune checkpoint blockade antibodies comprises a member selected from the group consisting of ipilimumab (anti-CTLA4), pembrolizumab (anti-PD1), nivolumab (anti-PD1), and atezolizumab (anti-PD-L1).

88. The method of any one of claims 84 to 87, wherein the targeting ligands comprise melanocortin-1 receptor (MC1-R) targeting ligands of a single type or multiple types.

89. The method of any one of claims 84 to 88, wherein resistance to immune ICB is limited by combining particle-driven ferroptosis and enhanced pro-inflammatory responses with one or more immune checkpoint blockade antibodies and/or selective PI3Kγ-targeting to subvert immunosuppressive components in a tumor microenvironment (TME).

90. A method comprising administering to a subject (a) a nanoparticle composition in a unit dosage effective for enhanced treatment of cancer (e.g., prostate cancer), wherein the subject has been diagnosed with the cancer and is receiving (or has received) therapy with one or more immune checkpoint blockade antibodies, the nanoparticle composition comprising ultrasmall nanoparticles, said nanoparticles comprising targeting ligands; and (b) external beam radiotherapy or molecular radiotherapy, e.g., peptide radioligands (e.g., radiolabeled PSMA-targeting ligands).

91. The method of claim 90, comprising administering to the subject molecular radiotherapy, wherein the molecular radiotherapy comprises a radiotherapeutic label.

92. The method of claim 91, wherein the radiotherapeutic label comprises an alpha-emitting radioisotope or a beta-emitting radioisotope.

93. The method of claim 92, wherein the alpha-emitting radioisotope comprises ²²Ac.

94. The method of claim 93, wherein the beta-emitting radioisotope comprises ¹⁷Lu.

95. The method of any one of claims 90 to 94, wherein the one or more immune checkpoint blockade antibodies comprises a member selected from the group consisting of ipilimumab (anti-CTLA4), pembrolizumab (anti-PD1), nivolumab (anti-PD1), and atezolizumab (anti-PD-L1).

96. The method of any one of claims 90 to 95, wherein the targeting ligands comprise PSMA-targeting ligands of a single type or multiple types.

97. The method of any one of claim 90 to 96, further comprising administering to the subject a myeloid-targeting inhibitor.

98. The method of claim 97, wherein the myeloid-targeting inhibitor comprises a PI3Kγ-selective inhibitor targeting myeloid cells (e.g., IPI-549).

99. The method of any one of claims 80 to 98, wherein the nanoparticles have a diameter no greater than 20 nm.

100. The method of any one of claims 80 to 98, wherein the nanoparticles have a diameter no greater than 10 nm.

101. The method of any one of claims 80 to 100, wherein the nanoparticles comprise silica.

102. The method of any one of claims 80 to 101, wherein each of the nanoparticles comprises 1 to 25 targeting ligands.

103. The method of any one of claims 80 to 102, wherein the cancer comprises prostate cancer, ovarian cancer (e.g., high-grade ovarian cancer), malignant brain tumors, or melanoma.
